(19) United States
(12) Patent Application Publication
(10) Pub. No.: US 2006/0173015 A1
(43) Pub. Date: Aug. 3, 2006

(54) Cyclohexyldiamines as selective alpha 1a/1d adrenoreceptor antagonists for the treatment of benign prostatic hypertrophy and lower urinary tract symptoms

(76) Inventors: George Chiu, Bridgewater, NJ (US); Shengjian Li, Belle Mead, NJ (US); Peter J. Connolly, New Providence, NJ (US); Virginia L. Pulito, Flemington, NJ (US); Jessica J. Liu, Three Bridges, NJ (US); Steven A. Middleton, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/262,551

(22) Filed: Oct. 28, 2005

Related U.S. Application Data
(60) Provisional application No. 60/623,609, filed on Oct. 29, 2004.

(51) Int. Cl. A61K 31/495; A61K 31/496; C07D 403/02; C07D 241/04

(52) U.S. Cl. 514/252.13; 514/255.03; 544/386; 544/359

(57) ABSTRACT

The present invention relates to compounds of Formula (I) 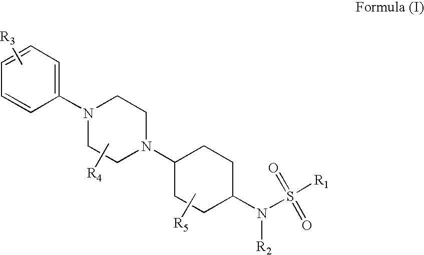
or a pharmaceutically acceptable form thereof, as dual selective α₁ₐ/α_{1d} adrenoreceptor antagonists for the treatment of benign prostatic hypertrophy and lower urinary tract symptoms. The present invention also relates to pharmaceutical compositions comprising said new compounds, new processes to prepare these new compounds and new uses as a medicine as well as method of treatments.

### CROSS REFERENCE TO RELATED APPLICATIONS

This present application claims benefit of U.S. Provisional Patent Application Ser. No. 60/623,609, filed Oct. 29, 2004, which is incorporated herein by reference in its entirety and for all purposes.

### FIELD OF THE INVENTION

The present invention relates to new compounds, more particularly new cyclohexyldiamines as selective α₁ₐ/α_{1d} adrenoreceptor antagonists for the treatment of benign prostatic hypertrophy and/or lower urinary tract symptoms. The present invention also relates to pharmaceutical compositions comprising said new compounds, new processes to prepare these new compounds, to the use of these compounds as α₁ₐ/α_{1d} adrenoreceptor modulators and new uses as a medicine as well as method of treatments.

### RELATED ART

The adrenergic receptors (ARs), through which norepinephrine and epinephrine exert their biological activities, are targets for many therapeutically important drugs. The α₁-ARs play a dominant role in control of smooth muscle contraction and are important in control of blood pressure, nasal congestion, prostate function, and other processes (Harrison et al., Trends Pharmacol Sci; 1991; 62-67). The α₁-ARs were originally classified by pharmacological profiling into two subtypes, α₁ₐ and α_{1b} (Morrow and Creese, Mol. Pharmacol; 1986; 29: 231-330; Minneman et al., Mol. Pharmacol; 1988; 33:509-514). Three genes encoding different α₁-AR subtypes (α₁ₐ, α_{1b}, and α_{1d}) have been cloned for a number of species, including human (Schwinn et al., J. Biol Chem; 1990; 265: 8183-8189; Ramarao et al., J Biol Chem; 1992; 267:21936-21945; Bruno et al., Biochem Biophys Res Commnun; 1991; 179: 1485-1490). These three cloned α₁-ARs are best differentiated from one another on the basis of the relative binding affinities of a series of antagonist compounds. There is general agreement that the α₁ₐ- and α_{1b}-ARs correspond to the pharmacologically defined α₁ₐ- and α_{1b}-ARs, while the functional role of the α_{1d}-AR is less clear, although it appears to mediate contraction of certain blood vessels (Goetz et al., Eur J Pharmacol; 1991; 272:R5-R6). Like other ARs, the α₁-ARs are members of the G-protein coupled receptor super family, and in most cells the primary functional response to activation of all α₁-AR subtypes is an increase in intracellular Ca²⁺.

Benign prostatic hyperplasia (BPH) is a non-malignant enlargement of the prostate and is the cause of lower urinary tract symptoms (LUTS) in a large segment of the elderly male population. Symptoms such as straining, hesitancy, dribbling, weak stream, and incomplete emptying are classified as voiding or obstructive symptoms. Obstructive symptoms are primarily due to pressure upon the urethra from the physical mass of the enlarged prostate gland (the static component) and the increased tone of the smooth muscle of the prostate stroma and bladder neck (the dynamic component) (Caine, J Urol; 1986; 136: 1-4). Irritative or storage symptoms associated with BPH are frequency, urgency, nocturia, dysuria, and burning sensation. Patients feel that these symptoms are more disturbing than the obstructive symptoms. As the urine flow is reduced, due to the bladder outlet obstruction, the wall around the bladder base thickens and becomes hyperactive.

Functional studies have established that prostate smooth muscle tone is maintained through α₁-ARs and that these receptors mediate the dynamic component of obstruction. α₁-AR antagonists have successfully been used to treat the obstructive symptoms associated with BPH (Jardin et al., Scientific Communications Int; 1998; pp 559-632). Furthermore, the α₁ₐ-AR subtype comprises the majority of α₁-ARs in human prostatic smooth muscle and has been shown to mediate contraction in this tissue. Originally introduced as antihypertensive agents, α₁-AR antagonists have become increasingly important in the management of BPH. α₁-AR antagonists reduce smooth muscle tone in the prostate and lower urinary tract, thereby relaxing the bladder outlet and increasing urinary flow. The major disadvantage of non-selective α₁-blockers is their adverse effect profile, particularly vasodilatation leading to dizziness, postural hypotension, asthenia, and occasionally syncope. For this reason, it would be desirable to block α₁-ARs in the lower urinary tract without antagonizing the α₁-ARs responsible for maintaining vascular tone.

A number of factors can be involved in lower urinary tract symptoms. Adrenergic stimulation of the bladder results in relaxation due to β-ARs, which dominate over contraction-mediating α₁-ARs. Bladder contraction is primarily mediated by muscarinic receptors. Some studies indicate that the contribution from α₁-ARs increases in hyperactive bladders due to bladder outlet obstruction or other conditions (Perlberg et al., Urology; 1982; 20:524-527); Restorick and Mundy, Br J Urol; 1989; 63: 32-35). However another study finds no change in α₁-AR receptor function between normal and hypertrophic bladder due to outlet obstruction (Smith and Chapple, Neurolog Urodyn; 1994; 12: 414-415). It remains unclear, which α₁-AR is dominant in the human bladder. One study reported a predominance of the α₁ₐ subtype mRNA in the bladder dome, base, and trigone (Walden et al., J Urol; 1997; 157: 414-415). Another report found that the α_{1d} subtype is present as 66% of the α₁-ARs at both the mRNA and protein levels, while the α₁ₐ subtype is present as 34% of the total, with no evidence of the α_{1b} subtype (Malloy et al., J Urol; 1998; 160: 937-943). Drugs that selectively antagonize only the α₁ₐ-AR subtype appear to have little effect upon the irritative symptoms of BPH. Ro-70004, a α₁ₐ subtype-selective compound was reported to be discontinued in clinical studies when it was found to have poor efficacy in treating these symptoms (Blue et al., Abstract 5^{th} International Consultation on BPH (Jun. 25-28) 2000). α_{1d}-ARs may be involved in mediating the irritative symptoms; however, the location of these α_{1d}-ARs is unknown (Piascik and Perez, J Pharmacol Exp Ther; 2001; 298: 403-410).

Studies have demonstrated Central Nervous Systems (CNS) inhibitory effects of α₁ antagonists upon the sympathetic and somatic outflow to the bladder in cats (Danuser and Thor, J Urol; 1995; 153: 1308-1312; Ramage and Wyllie, Eur J Pharmacol; 1995; 294: 645-650). Intrathecally administered doxazosin caused a decrease in micturition pressure in both normal rats and rats with bladder hypertrophy secondary to outlet obstruction (Ishizuka et al., Br J Pharmacol; 1996; 117:962-966). These effects may be due to a reduction in parasympathetic nerve activity in the spinal cord and ganglia. Other studies used spontaneously hypertensive rats, which have overactive bladders, to demonstrate that α₁-AR antagonism only given intrathecally caused a return to normal micturition (Persson et al., Am J Physiol; 1998; 275:R1366-1373, Steers et al. 1999; Exp Physiol; 84:137-147.). Antagonists administered intra-arterially near the bladder, or ablation of peripheral noradrenergic nerves, had no effect upon the bladder overactivity in these animals, indicating that α₁-ARs in the spinal cord control the bladder activity. Spinal α₁-ARs may be important targets for pharmacological treatment of BPH symptoms in humans as well. All three α₁-AR subtype mRNAs are found throughout the human spinal cord, however the α_{1d} subtype mRNA is present at twice the level of the other subtypes, particularly in the ventral sacral motor neurons and autonomic parasympathetic pathways. (Stafford-Smith et al., Mol Brain Res; 1998; 63:234-261). There may be clinical advantages to the pharmacological blockade of the α_{1d}-ARs in the CNS in reducing BPH symptoms.

Antagonism of α_{1d}-ARs in the CNS and bladder may be an important activity in reducing the irritative or filling symptoms of BPH and improving patient symptom scores. Tamsulosin (Flomax®, Yamanuchi and Boehringer Ingelheim) is a α₁-AR antagonist, which is about 15-fold selective for the α₁ₐ and α_{1d} subtypes over the α_{1b} subtype. Large clinical trials of BPH patients with tamsulosin showed improvement in both obstructive and irritative symptoms, however, cardiovascular and erectile dysfunction side effects were seen (Abrams et al. Br J Urol; 1995; 76:325-336; Chapple et al., Eur Urol; 1996; 29:155-167; Lepor, Urology; 1998; 51:892-900). Patients treated with non-selective α₁ antagonists also have improvement in both obstructive and irritative symptoms, although the risk of vascular side effects is greater. Generally, the α₁ₐ subtype predominates in arteries at the mRNA and protein levels, while all three subtypes are found in veins. The particular vessel bed is important in that the α₁ₐ is the subtype found primarily in the splanchnic and coronary arteries, while the α_{1d} subtype is the predominant subtype found in the aorta. The α₁-AR subtypes in the vasculature have been found to change with age. Contraction of the mammary artery is mediated by both α₁ₐ and α_{1b} subtypes. The number of at receptors in the mammary artery doubles with age; however, the α_{1b} subtype increases to a greater extent than the α₁ₐ subtype (Raudner et al., Circulation; 1999; 100:2336-2343). The α_{1b} subtype may play a greater role in vascular tone in elderly patients. This suggests that an α₁ₐ and α_{1d}-selective antagonist may have less effects upon the vasculature in elderly BPH patients, resulting in fewer cardiovascular side effects than are seen with non-selective α₁ antagonists, but provide relief from both obstructive and irritative symptoms.

A uroselective, cardiovascular-sparing α₁-AR antagonist would be expected to provide symptomatic relief of BPH comparable to currently marketed non-selective agents such as terazosin/Hytrin®, doxazosin/Cardura®, alfuzosin/Xatral®/Uroxatral® and weakly selective tamsulosin/Flomax®/Harnal®, without the undesirable side effects of postural hypotension, dizziness, and syncope. Ejaculatory dysfunction, or retrograde ejaculation, is a side effect seen in 10 to 35% of patients using tamsulosin (Lepor, Urology; 1998; 51:901-906; Andersson and Wyllie, Brit J Urol Int; 2003; 92:876-877). This activity has been attributed to tamsulosin antagonism at the 5-HT₁ₐ receptor. This often leads to discontinuation of treatment. Furthermore, the non-selective α₁-AR antagonists and tamsulosin are contraindicated for use in conjunction with PDE inhibitors. There is likely to be high co-morbidity between LUTS and erectile dysfunction patients. Patients being treated for LUTS with the current α₁-AR blockers will find that they are excluded from using PDE inhibitors. An α₁-AR antagonist with a receptor subtype binding profile, which is selective for the α₁ₐ and α_{1d}, subtypes, but with relatively little antagonism of the α_{1b} subtype may effectively treat both obstructive and irritative symptoms of BPH. Such a compound is likely to have a low cardiovascular side effect profile and allow for use in conjunction with PDE inhibitors. Also low binding activity at the 5-HT_{1A} receptor is likely to reduce the incidence of ejaculatory side effects.

LUTS also develop in women of a certain age. As in men, LUTS in women include both filling symptoms such as urgency, incontinence and nocturnia, and voiding symptoms such as weak stream, hesitancy, incomplete bladder emptying and abdominal straining. The presence of this condition both in men and women suggests that at least part of the aetiology may be similar in the two sexes.

Accordingly, there is a need to provide dual selective α₁ₐ/α_{1d} adrenoreceptor antagonists, in other words compounds that interact both with the α₁ₐ and α_{1d} receptor but do not interact (or at least interact substatntially less) with the α_{1b} receptor. The compounds of this invention are believed to be more efficacious drugs mainly for BPH/LUTS patients, and at the same time these compounds should show less unwanted side effects than the existing pharmaceuticals.

### SUMMARY OF THE INVENTION

The present invention provides a 4-phenyl-piperazin-1-yl substituted cyclohexyl sulfonamide compound of Formula (I) 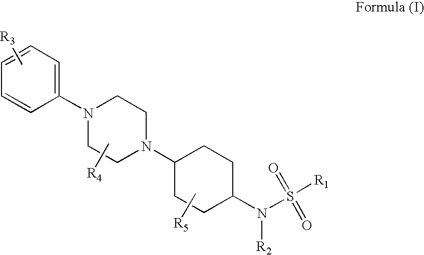
and pharmaceutically acceptable forms thereof, wherein
R₁ is selected from the group consisting of
(1) aryl,
(2) C₁₋₈alkyl(aryl),
(3) C₃₋₈cycloalkyl,
(4) C₁₋₈alkyl(C₃₋8cycloalkyl),
(5) heteroaryl,
(6) C₁₋₈alkyl(heteroaryl),
(7) heterocyclyl, and
(8) C₁₋₈alkyl(heterocyclyl),
wherein (1), (3), (5) and (7) and the aryl, C₃₋₈cycloalkyl, heteroaryl and heterocyclyl portions of (2), (4), (6) and (8) respectively are optionally substituted with up to four substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy) 1-3,
(vii) CO₂(C₁₋₈alkyl),
(viii) SO₂ substituted on sulfur with a substituent selected from the group consisting of C₁₋₈alkyl, C₃₋₈cycloalkyl, aryl, heteroaryl, or heterocyclyl,
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl),
(xvi) C₁₋₈alkoxy(aryl),
(xvii) C₁₋₈alkyl(heteroaryl),
(xviii) C₁₋₈alkyl(heterocyclyl);
(xix) CO substituted on carbon with a substituent selected from the group consisting of hydrogen, C₁₋₈alkyl, C₃₋₈cycloalkyl, aryl, heteroaryl, or heterocyclyl,
(xx) SO substituted on sulfur with a substituent selected from the group consisting of C₁₋₈alkyl, C₃₋₈cycloalkyl, aryl, heteroaryl, or heterocyclyl,
(xxi) C(O)N substituted on nitrogen with two substituents selected from the group consisting of hydrogen, C₁₋₈alkyl, C₃₋₈cycloalkyl, aryl, heteroaryl, or heterocyclyl,
(xxii) SO₂N substituted on nitrogen with two substituents selected from the group consisting of hydrogen, C₁₋₈alkyl, C₃₋₈cycloalkyl, aryl, heteroaryl, or heterocyclyl,
(xxiii) NHSO₂ substituted on sulfur with a substituent selected from the group consisting of C₁₋₈alkyl, C₃₋₈cycloalkyl, aryl, heteroaryl, or heterocyclyl,
(xxiv) NH(CO) substituted on carbon with a substituent selected from the group consisting of hydrogen, C₁₋₈alkyl, C₃₋₈cycloalkyl, aryl, heteroaryl, or heterocyclyl,
(xxv) NHSO₂N substituted on nitrogen with two substituents selected from the group consisting of hydrogen, C₁₋₈alkyl, C₃₋₈cycloalkyl, aryl, heteroaryl, or heterocyclyl,
(xxvi) NH(CO)N substituted on nitrogen with two substituents selected from the group consisting of hydrogen, C₁₋₈alkyl, C₃₋₈cycloalkyl, aryl, heteroaryl, or heterocyclyl,
(xxvii) C₃₋₈cycloalkyl,
(xxviii) aryl,
(xxix) heteroaryl, and
(xxx) heterocyclyl;
R₂ is selected from the group consisting of hydrogen and C₁₋₈alkyl;
R₃ is up to four optionally present substituents independently selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂ substituted on sulfur with a substituent selected from the group consisting of C₁₋₈alkyl, C₃₋₈cycloalkyl, aryl, heteroaryl, or heterocyclyl,
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl),
(25) CO substituted on carbon with a substituent selected from the group consisting of hydrogen, C₁₋₈alkyl, C₃₋₈cycloalkyl, aryl, heteroaryl, or heterocyclyl,
(26) SO substituted on sulfur with a substituent selected from the group consisting of C₁₋₈alkyl, C₃₋₈cycloalkyl, aryl, heteroaryl, or heterocyclyl,
(27) SO₂ substituted on sulfur with a substituent selected from the group consisting of C₁₋₈alkyl, C₃₋₈cycloalkyl, aryl, heteroaryl, or heterocyclyl,
(28) C(O)N substituted on nitrogen with two substituents selected from the group consisting of hydrogen, C₁₋₈alkyl, —C₃₋₈cycloalkyl, aryl, heteroaryl, or heterocyclyl,
(29) SO₂N substituted on nitrogen with two substituents selected from the group consisting of hydrogen, C₁₋₈alkyl, C₃₋₈cycloalkyl, aryl, heteroaryl, or heterocyclyl,
(30) NHSO₂ substituted on sulfur with a substituent selected from the group consisting of C₁₋₈alkyl, C₃₋₈cycloalkyl, aryl, heteroaryl, or heterocyclyl,
(31) NH(CO) substituted on carbon with a substituent selected from the group consisting of hydrogen, C₁₋₈alkyl, C₃₋₈cycloalkyl, aryl, heteroaryl, or heterocyclyl,
(32) NHSO₂N substituted on nitrogen with two substituents selected from the group consisting of hydrogen, C₁₋₈alkyl, C₃₋₈cycloalkyl, aryl, heteroaryl, or heterocyclyl,
(33) NH(CO)N substituted on nitrogen with two substituents selected from the group consisting of hydrogen, C₁₋₈alkyl, C₃₋₈cycloalkyl, aryl, heteroaryl, or heterocyclyl, and
(34) C₃₋₈cycloalkoxy;
wherein (15), (18), (21) and (23) and the aryl, C₃₋₈cycloalkyl, heteroaryl and heterocyclyl portions of (8), (16), (17), (19), (20), (22), (24), (25), (26), (27), (28), (29), (30), (31), (32), (33), and (34) are optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro, and
(xiv) C₁₋₈alkyl(amino) optionally mono- or di-substituted on amino with C₁₋₈alkyl; and
R₄ and R₅ are up to two optionally present substituents independently selected from the group consisting of oxo and C₁₋₆alkyl.

The present invention also provides pharmaceutical compositions comprising a therapeutically effective amount of any of the compounds of Formula (I) described in the present application and a pharmaceutical acceptable carrier. An example of the invention is a pharmaceutical composition made by combining any of the compounds of Formula (I) described in the present application and a pharmaceutically acceptable carrier. Another illustration of the invention is a process for making a pharmaceutical composition comprising combining any of the compounds described in the present application and a pharmaceutically acceptable carrier.

It is an aspect of the present invention to provide α₁ₐ/α_{1d} adrenoceptor modulators, more specifically inhibitors thereof, more interestingly antagonists thereof. The compounds of the present invention are preferably selective dual α₁ₐ/α_{1d} adrenoceptor modulators, more specifically inhibitors thereof, more interestingly antagonists thereof.

In another aspect, the invention is directed to methods for preventing contractions of the prostate, bladder and other organs of the lower urinary tract without substantially affecting blood pressure, by administering a compound of Formula (I) described in the present application or a pharmaceutical form comprising it to a mammal (including a human) suffering from contractions of the bladder and other organs of the lower urinary tract in an amount effective for the particular use.

A further object of the present invention is a method of treatment of a patient suffering from Benign Prostatic Hyperplasia (BPH), the method comprising administering an effective amount of a compound of Formula (I) described in the present application or a pharmaceutical form comprising it to a patient suffering from BPH.

A further object of the present invention is a method for the treatment of lower-urinary-tract-symptoms (LUTS), which include, but are not limited to, filling symptoms, urgency, incontinence and nocturia, as well as voiding problems such as weak stream, hesitancy, intermnittency, incomplete bladder emptying and abdominal straining, the method comprising administering an effective amount of a compound of Formula (I) described in the present application or a pharmaceutical form comprising it to a patient in need of such treatment.

A further object of the present invention is the use of these compounds as a medicine.

Yet another object of the present invention is the use of a compound of the present invention for the manufacture of a medicament for treating BPH and/or LUTS.

Still another object of the present invention is a method for treating of BPH and/or LUTS, the method comprising administering a therapeutically effective amount of a compound of the present invention in combination with an effective amount of a 5α-reductase, such as, for example, finasteride or durasteride.

Still another object of the present invention is method for treating of BPH and/or LUTS, the method comprising administering a therapeutically effective amount of a compound of the present invention in combination with a therapeutically effective amount of a NK-1 inhibitor.

It is still another object of the present invention to provide methods for treating of BPH and/or LUTS, the method comprising administering an therapeutically effective amount of a compound of the present invention in combination with a therapeutically effective amount of anti-antiandrogens, androgen receptor antagonists, selective androgen receptor modulators, a PDE inhibitor, urinary incontinence drugs (e.g. anti-muscarinics) or 5HT-receptor modulators.

### DETAILED DESCRIPTION OF THE INVENTION

It should be understood that all compounds described and listed herein are meant to include all hydrates, solvates, polymorphs and pharmaceutically acceptable salts thereof. It should also be understood that unless otherwise indicated compounds of Formula (I) are meant to comprise the stereochemically isomeric forms thereof.

The present invention provides a 4-phenyl-piperazin-1-yl substituted cyclohexyl sulfonamide compound of Formula (I) 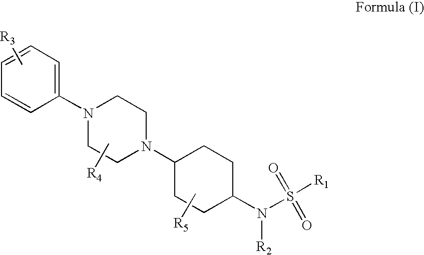
and pharmaceutically acceptable forms thereof, wherein
R₁ is selected from the group consisting of
(1) aryl,
(2) C₁₋₈alkyl(aryl),
(3) C₃₋₈cycloalkyl,
(4) C₁₋₈alkyl(C₃₋₈cycloalkyl),
(5) heteroaryl,
(6) C₁₋₈alkyl(heteroaryl),
(7) heterocyclyl, and
(8) C₁₋₈alkyl(heterocyclyl),
wherein (1), (3), (5) and (7) and the aryl, C₃₋₈cycloalkyl, heteroaryl and heterocyclyl portions of (2), (4), (6) and (8) respectively are optionally substituted with up to four substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂ substituted on sulfur with a substituent selected from the group consisting of C₁₋₈alkyl, C₃₋₈cycloalkyl, aryl, heteroaryl, or heterocyclyl,
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl),
(xvi) C₁₋₈alkoxy(aryl),
(xvii) C₁₋₈alkyl(heteroaryl),
(xviii) C₁₋₈alkyl(heterocyclyl);
(xix) CO substituted on carbon with a substituent selected from the group consisting of hydrogen, C₁₋₈alkyl, C₃₋₈cycloalkyl, aryl, heteroaryl, or heterocyclyl,
(xx) SO substituted on sulfur with a substituent selected from the group consisting of C₁₋₈alkyl, C₃₋₈cycloalkyl, aryl, heteroaryl, or heterocyclyl,
(xxi) C(O)N substituted on nitrogen with two substituents selected from the group consisting of hydrogen, C₁₋₈alkyl, C₃₋₈cycloalkyl, aryl, heteroaryl, or heterocyclyl,
(xxii) SO₂N substituted on nitrogen with two substituents selected from the group consisting of hydrogen, C₁₋₈alkyl, C₃₋₈cycloalkyl, aryl, heteroaryl, or heterocyclyl,
(xxiii) NHSO₂ substituted on sulfur with a substituent selected from the group consisting of C₁₋₈alkyl, C₃₋₈cycloalkyl, aryl, heteroaryl, or heterocyclyl,
(xxiv) NH(CO) substituted on carbon with a substituent selected from the group consisting of hydrogen, C₁₋₈alkyl, C₃₋₈cycloalkyl, aryl, heteroaryl, or heterocyclyl,
(xxv) NHSO₂N substituted on nitrogen with two substituents selected from the group consisting of hydrogen, C₁₋₈alkyl, C₃₋₈cycloalkyl, aryl, heteroaryl, or heterocyclyl,
(xxvi) NH(CO)N substituted on nitrogen with two substituents selected from the group consisting of hydrogen, C₁₋₈alkyl, C₃₋₈cycloalkyl, aryl, heteroaryl, or heterocyclyl,
(xxvii) C₃₋₈cycloalkyl,
(xxviii) aryl,
(xxix) heteroaryl, and
(xxx) heterocyclyl;
R₂ is selected from the group consisting of hydrogen and C₁₋₈alkyl;
R₃ is up to four optionally present substituents independently selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂ substituted on sulfur with a substituent selected from the group consisting of C₁₋₈alkyl, C₃₋₈cycloalkyl, aryl, heteroaryl, or heterocyclyl,
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl,
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl),
(25) CO substituted on carbon with a substituent selected from the group consisting of hydrogen, C₁₋₈alkyl, C₃₋₈cycloalkyl, aryl, heteroaryl, or heterocyclyl,
(26) SO substituted on sulfur with a substituent selected from the group consisting of C₁₋₈alkyl, C₃₋₈cycloalkyl, aryl, heteroaryl, or heterocyclyl,
(27) SO₂ substituted on sulfur with a substituent selected from the group consisting of C₁₋₈alkyl, C₃₋₈cycloalkyl, aryl, heteroaryl, or heterocyclyl,
(28) C(O)N substituted on nitrogen with two substituents selected from the group consisting of hydrogen, C₁₋₈alkyl, C₃₋₈cycloalkyl, aryl, heteroaryl, or heterocyclyl,
(29) SO₂N substituted on nitrogen with two substituents selected from the group consisting of hydrogen, C₁₋₈alkyl, C₃₋₈cycloalkyl, aryl, heteroaryl, or heterocyclyl,
(30) NHSO₂ substituted on sulfur with a substituent selected from the group consisting of C₁₋₈alkyl, C₃₋₈cycloalkyl, aryl, heteroaryl, or heterocyclyl,
(31) NH(CO) substituted on carbon with a substituent selected from the group consisting of hydrogen, C₁₋₈alkyl, C₃₋₈cycloalkyl, aryl, heteroaryl, or heterocyclyl,
(32) NHSO₂N substituted on nitrogen with two substituents selected from the group consisting of hydrogen, C₁₋₈alkyl, C₃₋₈cycloalkyl, aryl, heteroaryl, or heterocyclyl,
(33) NH(CO)N substituted on nitrogen with two substituents selected from the group consisting of hydrogen, C₁₋₈alkyl, C₃₋₈cycloalkyl, aryl, heteroaryl, or heterocyclyl, and
(34) C₃₋₈cycloalkoxy;
wherein (15), (18), (21) and (23) and the aryl, C₃₋₈cycloalkyl, heteroaryl and heterocyclyl portions of (8), (16), (17), (19), (20), (22), (24), (25), (26), (27), (28), (29), (30), (31), (32), (33), and (34) are optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro, and
(xiv) C₁₋₈alkyl(amino) optionally mono- or di-substituted on amino with C₁₋₈alkyl; and
R₄ and R₅ are up to two optionally present substituents independently selected from the group consisting of oxo and C₁₋₆alkyl.

Embodiments of a compound of Formula (I) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with up to four substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl),
(xvi) C₁₋₈alkoxy(aryl)
(xvii) C₁₋₈alkyl(heteroaryl), and
(xviii) C₁₋₈alkyl(heterocyclyl);
R₂ is selected from the group consisting of hydrogen and C₁₋₈alkyl; and
R₃ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl,
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl), and
(25) C₃₋₈cycloalkoxy.

Embodiments of a compound of Formula (I) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono or disubstituted with C₁₋₈alkyl,
(x) cyano,
(xi) halogen,
(xii) hydroxy,
(xiii) nitro,
(xiv) C₁₋₈alkyl(amino) optionally mono or disubstituted on amino with C₁₋₈alkyl,
(xv) C₁₋₈alkyl(aryl), and
(xvi) C₁₋₈alkoxy(aryl);
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₃,
(3) halogen, and
(4) C₃₋₈cycloalkoxy.

Embodiments of a compound of Formula (I) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro;
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₃,
(3) halogen, and
(4) C₃₋₈cycloalkoxy.

Embodiment of compounds of Formula (I) include compounds of Formula (Ia) 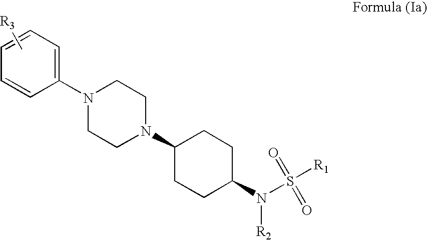
and pharmaceutically acceptable forms thereof, wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl), and
(xvi) C₁₋₈alkoxy(aryl);
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₁₇,
(3) halogen, and
(4) C₃₋₈cycloalkoxy.

Embodiments of a compound of Formula (Ia) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl), and
(xvi) C₁₋₈alkoxy(aryl);
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₃,
(3) halogen, and
(4) C₃₋₈cycloalkoxy.

Embodiments of a compound of Formula (Ia) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(ii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro;
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₃,
(3) halogen, and
(4) C₃₋₈cycloalkoxy.

Embodiment of compounds of Formula (I) include compounds of Formula (Ib) 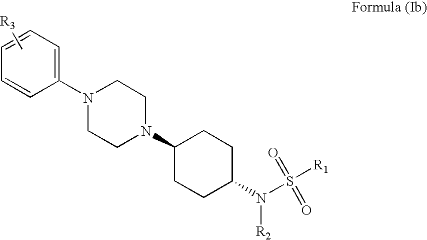
and pharmaceutically acceptable forms thereof, wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl), and
(xvi) C₁₋₈alkoxy(aryl);
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₁₇,
(3) halogen, and
(4) C₃₋₈cycloalkoxy.

Embodiments of a compound of Formula (Ib) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono or disubstituted with C₁₋₈alkyl,
(x) cyano,
(xi) halogen,
(xii) hydroxy,
(xiii) nitro,
(xiv) C₁₋₈alkyl(amino) optionally mono or disubstituted on amino with C₁₋₈alkyl,
(xv) C₁₋₈alkyl(aryl), and
(xvi) C₁₋₈alkoxy(aryl);
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₃,
(3) halogen, and
(4) C₃₋₈cycloalkoxy.

Embodiments of a compound of Formula (Ib) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of

(i) C₁₋₈alkyl,
(ii) C₁₋₈alkoxy,
(ii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro;
R₂ is selected from the group consisting of hydrogen and C₁₋₈alkyl; and
R₃ is selected from the group consisting of
(1) C₁₋₈alkoxy,
(2) C₁₋₈alkoxy(halogen)₁₋₃,
(3) halogen, and
(4) C₃₋₈cycloalkoxy.

Embodiments of compounds of Formula (I) include compounds of Formula (II) 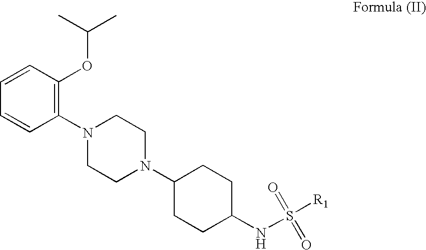
and pharmaceutically acceptable forms thereof, wherein
R₁ is selected from the group consisting of
(1) aryl,
(2) C₁₋₈alkyl(aryl),
(3) C₃₋₈cycloalkyl,
(4) C₁₋₈alkyl(C₃₋₈cycloalkyl),
(5) heteroaryl,
(6) C₁₋₈alkyl(heteroaryl),
(7) heterocyclyl, and
(8) C₁₋₈alkyl(heterocyclyl),
wherein (1), (3), (5) and (7) and the aryl, C₃₋₈cycloalkyl, heteroaryl and heterocyclyl portions of (2), (4), (6) and (8) respectively are optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono or disubstituted on amino with C₁₋₈alkyl,
(xv) C₁₋₈alkyl(aryl), and
(xvi) C₁₋₈alkoxy(aryl).

Embodiments of a compound of Formula (II) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl), and
(xvi) C₁₋₈alkoxy(aryl).

Embodiments of compounds of Formula (II) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(iii) C₁₋₈alkyl(halogen)₁₋₁₇,
(iv) C₁₋₈alkoxy(halogen)₁₋₁₇,
(v) C₁₋₈alkyl(hydroxy)₁₋₃,
(vi) SO₂(C₁₋₈alkyl),
(vii) amino optionally mono- or di-substituted with C₁₋₈alkyl,
(viii) cyano,
(ix) halogen,
(x) hydroxy,
(xi) nitro,
(xii) C₁₋₈alkyl(amino) optionally mono- or di-substituted on amino with C₁₋₈alkyl,
(xiii) C₁₋₈alkyl(aryl), and
(xiv) C₁₋₈alkoxy(aryl).

Embodiments of a compound of Formula (II) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(ii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro.

An embodiment of compounds of Formula (II) include compounds of Formula (IIa) 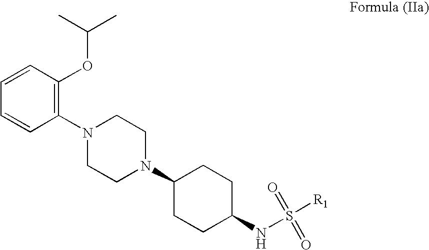
and pharmaceutically acceptable forms thereof, wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(ii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro.

Embodiments of a compound of Formula (IIa) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(ii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro.

An embodiment of a compound of Formula (IIa) includes a compound wherein R₁ is selected from

| Cpd | R₁ |
|---|---|
| 57 | CH₂-Ph |
| 58 | quinolin-8-yl |
| 59 | pyridin-3-yl |
| 60 | (4-OMe)-pyridin-3-yl |
| 61 | (5-Br-6-Cl)-pyridin-3-yl |

An embodiment of a compound of Formula (II) includes a compound of Formula (IIb) 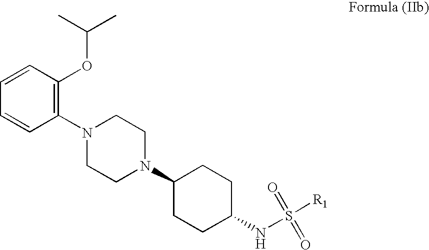
and pharmaceutically acceptable forms thereof, wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(ii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro.

Embodiments of compounds of Formula (IIb) include compounds wherein
R₁ is selected from the group consisting of
(1) aryl,
(2) C₁₋₈alkyl(aryl), and
(3) heteroaryl,
wherein (1) and (3) and the aryl portion of (2) is optionally substituted with from one to two substituents independently selected from the group consisting of
(i) C₁₋₈alkyl,
(ii) C₁₋₈alkoxy,
(ii) C₁₋₈alkyl(halogen)₁₋₁₇,
(iv) C₁₋₈alkoxy(halogen)₁₋₁₇,
(v) SO₂(C₁₋₈alkyl),
(vi) cyano,
(vii) halogen, and
(viii) nitro.

Embodiments of compounds of Formula (IIb) includes compounds wherein R₁ is selected from

| Cpd | R₁ |
|---|---|
| 62 | CH₂-Ph |
| 63 | quinolin-8-yl |
| 64 | pyridin-3-yl |
| 65 | (4-OMe)-pyridin-3-yl |
| 66 | (5-Br-6-Cl)-pyridin-3-yl |

Embodiments of compounds of Formula (I) include compounds of Formula (III) 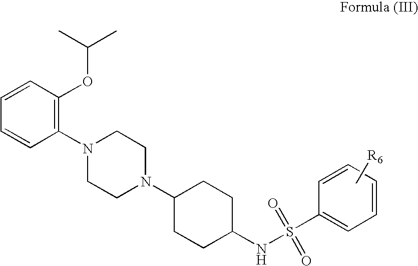
and pharmaceutically acceptable forms thereof, wherein
R₆ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) C₁₋₈alkyl(aryl), and
(16) C₁₋₈alkoxy(aryl).

Embodiments of a compound of Formula (III) include compounds wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro,
(12) C₁₋₈alkyl(amino) optionally mono- or di-substituted on amino with C₁₋₈alkyl,
(13) C₁₋₈alkyl(aryl), and
(14) C₁₋₈alkoxy(aryl).

Embodiments of compounds of Formula (III) include compounds wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro, and
(12) C₁₋₈alkyl(amino) optionally mono- or di-substituted on amino with C₁₋₈alkyl.

Embodiments of a compound of Formula (III) include compounds wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (III) include compounds of Formula (IIIa) 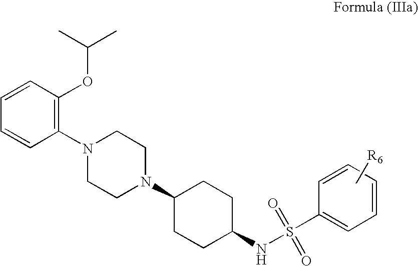
and pharmaceutically acceptable forms thereof, wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono or disubstituted with C₁₋₈alkyl,
(8) cyano,
(9) halogen,
(10) hydroxy,
(11) nitro,
(12) C₁₋₈alkyl(amino) optionally mono or disubstituted on amino with C₁₋₈alkyl,
(13) C₁₋₈alkyl(aryl), and
(14) C₁₋₈alkoxy(aryl).

Embodiments of compounds of Formula (IIIa) include compounds wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono or disubstituted with C₁₋₈alkyl,
(8) cyano,
(9) halogen,
(10) hydroxy,
(11) nitro, and
(12) C₁₋₈alkyl(amino) optionally mono- or di-substituted on amino with C₁₋₈alkyl.

Embodiments of compounds of Formula (IIIa) include compounds wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (IIIa) includes compounds wherein R₆ is selected from

| Cpd | R₆ |
|---|---|
| 1 | 2,4-diCl |
| 2 | 3-CF₃ |
| 3 | 5-Cl-2-OMe |
| 4 | 5-Me-2-OMe |
| 5 | 2-SO₂Me |
| 6 | 4-SO₂Me |
| 7 | 3,4-diOMe |
| 8 | 2,5-diOMe |
| 9 | 4-CF₃ |
| 10 | 4-F |
| 11 | 3-Cl-2-Me |
| 12 | 5-Cl-2-F |
| 13 | 2-CF₃ |
| 14 | 3-Cl-4-F |
| 15 | 5-F-2-OMe |
| 16 | 3-CF₃ |
| 17 | 4-OCF₃ |
| 18 | 4-Me-3-Cl |
| 19 | 5-NO₂-2-OMe |
| 20 | 5-CF₃-2-OMe |
| 21 | 2-F |
| 22 | 3-F |
| 23 | 3-Cl-2-F |
| 24 | 4-Cl-2-F |
| 25 | H |
| 26 | 2-NO₂ |
| 27 | 2-CN |
| 28 | 5-Br-2-OMe |

Embodiments of compounds of Formula (III) include compounds of Formula (IIIb) 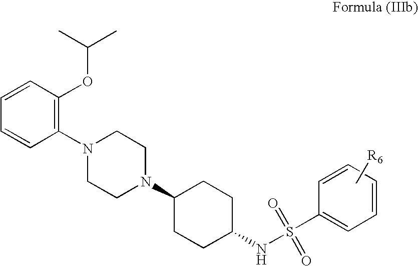
and pharmaceutically acceptable forms thereof, wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro,
(12) C₁₋₈alkyl(amino) optionally mono- or di-substituted on amino with C₁₋₈alkyl,
(13) C₁₋₈alkyl(aryl), and
(14) C₁₋₈alkoxy(aryl).

Embodiments of compounds of Formula (IIIb) include compounds wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro, and
(12) C₁₋₈alkyl(amino) optionally mono or disubstituted on amino with C₁₋₈alkyl.

Embodiments of compounds of Formula (IIIb) include compounds wherein
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (IIIb) include compounds wherein R₆ is selected from

| Cpd | R₆ |
|---|---|
| 29 | 2,4-diCl |
| 30 | 3-CF₃ |
| 31 | 5-Cl-2-OMe |
| 32 | 5-Me-2-OMe |
| 33 | 2-SO₂Me |
| 34 | 4-SO₂Me |
| 35 | 3,4-diOMe |
| 36 | 2,5-diOMe |
| 37 | 4-CF₃ |
| 38 | 4-F |
| 39 | 3-Cl-2-Me |
| 40 | 5-Cl-2-F |
| 41 | 2-CF₃ |
| 42 | 4-F-3-Cl |
| 43 | 5-F-2-OMe |
| 44 | 3-CF₃ |
| 45 | 4-OCF₃ |
| 46 | 4-Me-3-Cl |
| 47 | 5-NO₂-2-OMe |
| 48 | 5-CF₃-2-OMe |
| 49 | 2-F |
| 50 | 3-F |
| 51 | 3-Cl-2-F |
| 52 | 4-Cl-2-F |
| 53 | H |
| 54 | 2-NO₂ |
| 55 | 2-CN |
| 56 | 5-Br-2-OMe |

Embodiment of compounds of Formula (I) include a compound of Formula (IV) 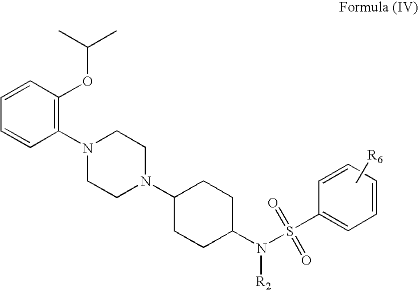
and pharmaceutically acceptable forms thereof, wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) C₁₋₈alkyl(aryl), and
(16) C₁₋₈alkoxy(aryl).

Embodiments of compounds of Formula (IV) include compounds wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro,
(12) C₁₋₈alkyl(amino) optionally mono- or di-substituted on amino with C₁₋₈alkyl,
(13) C₁₋₈alkyl(aryl), and

(14) C₁₋₈alkoxy(aryl).

Embodiments of compounds of Formula (IV) include compounds wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro, and
(12) C₁₋₈alkyl(amino) optionally mono- or di-substituted on amino with C₁₋₈alkyl.

Embodiments of a compound of Formula (IV) include compounds wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (IV) include compounds of Formula (IVa) 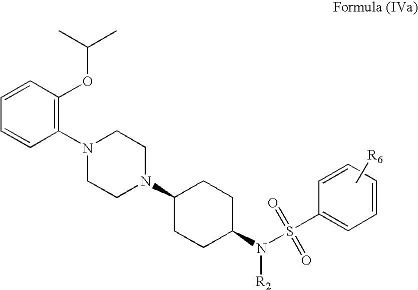
and pharmaceutically acceptable forms thereof, wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (IVa) include compounds wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (IVa) include compounds wherein R₂ and R₆ are dependently selected from

| Cpd | R₂ | R₆ |
|---|---|---|
| 67 | CH₂CH₃ | 3,4-diOMe |

Embodiments of compounds of Formula (IV) include compounds of Formula (IVb) 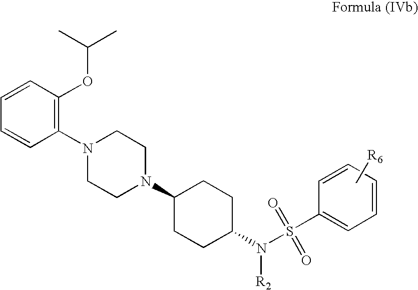
and pharmaceutically acceptable forms thereof, wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (IVb) include compounds wherein
R₂ is selected from the group consisting of hydrogen and C₁₋₈alkyl; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (IVb) include a compound wherein R₂ and R₆ are dependently selected from

| Cpd | R₂ | R₆ |
|---|---|---|
| 68 | CH₂CH₃ | 3,4-diOMe |

Embodiments of compounds of Formula (I) include compounds of Formula (V) 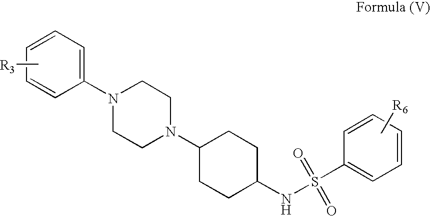
and pharmaceutically acceptable forms thereof, wherein
R₃ is up to four optionally present substituents independently selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl,
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl), and
(25) C₃₋₈cycloalkoxy,
wherein (15), (18), (21) and (23) and the aryl, C₃₋₈cycloalkyl, heteroaryl and heterocyclyl portions of (16), (17), (19), (20), (22), (24), and (25) are optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₁₇,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro, and
(xiv) C₁₋₈alkyl(amino) optionally mono- or di-substituted on amino with C₁₋₈alkyl; and
R₆ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) C₁₋₈alkyl(aryl), and
(14) C₁₋₈alkoxy(aryl).

Embodiments of compounds of Formula (V) include compounds wherein
R₃ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl,
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl), and
(25) C₃₋₈cycloalkoxy; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro,
(12) C₁₋₈alkyl(amino) optionally mono- or di-substituted on amino with C₁₋₈alkyl,
(13) C₁₋₈alkyl(aryl), and
(14) C₁₋₈alkoxy(aryl).

Embodiments of compounds of Formula (V) include compounds wherein
R₃ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) C₃₋₈cycloalkoxy; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) C₁₋₈alkyl(hydroxy)₁₋₃,
(6) SO₂(C₁₋₈alkyl),
(7) amino optionally mono- or di-substituted with C₁₋₈alkyl,
(8) cyano,
(9) halogen,
(10) hydroxy,
(11) nitro, and
(12) C₁₋₈alkyl(amino) optionally mono- or di-substituted on amino with C₁₋₈alkyl.

Embodiments of compounds of Formula (V) include compounds wherein
R₃ is selected from the group consisting of
(1) C₁₋₈alkoxy,
(2) C₁₋₈alkoxy(halogen)₁₋₃,
(3) halogen, and
(4) C₃₋₈cycloalkoxy; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (V) include compounds of Formula (Va) 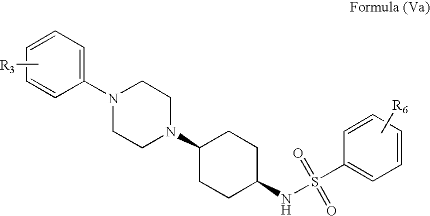
and pharmaceutically acceptable forms thereof, wherein
R₃ is selected from the group consisting of
(1) C₁₋₈alkoxy,
(2) C₁₋₈alkoxy(halogen)₁₋₁₇,
(3) halogen, and
(4) C₃₋₈cycloalkoxy; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (Va) include compounds wherein
R₃ is selected from the group consisting of
(1) C₁₋₈alkoxy,
(2) C₁₋₈alkoxy(halogen)₁₋₃,
(3) halogen, and
(4) C₃₋₈cycloalkoxy; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (Va) include compounds wherein R₃ and R₆ are dependently selected from

| Cpd | R₃ | R₆ |
|---|---|---|
| 69 | 2-OCH₂CF₃ | 3,4-diOMe |
| 71 | 4-F-2-OCH(CH₃)₂ | 3,4-diOMe |
| 73 | 4-F-2-OCH(CH₃)₂ | 5-Cl-2-OCH₃ |
| 74 | 4-F-2-OCH(CH₃)₂ | 5-Cl-2-F |
| 75 | 4-F-2-OCH₂CF₃ | 3,4-diOMe |
| 76 | 4-F-2-OCH₂CF₃ | 5-Cl-2-OCH₃ |
| 77 | 4-F-2-OCH₂CF₃ | 5-Cl-2-F |
| 83 | 5-F-2-OCH₂CF₃ | 3,4-diOMe |

Embodiments of compounds of Formula (V) include compounds of Formula (Vb) 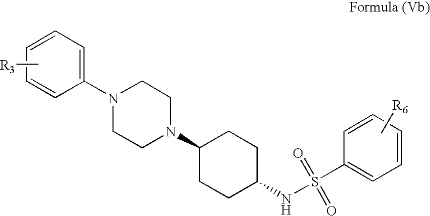
and pharmaceutically acceptable forms thereof, wherein
R₃ is selected from the group consisting of
(1) C₁₋₈alkoxy,
(2) C₁₋₈alkoxy(halogen)₁₋₁₇,
(3) halogen, and
(4) C₃₋₈cycloalkoxy; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of a compound of Formula (Vb) include compounds wherein
R₃ is selected from the group consisting of
(1) C₁₋₈alkoxy,
(2) C₁₋₈alkoxy(halogen)₁₋₃,
(3) halogen, and
(4) C₃₋₈cycloalkoxy; and
R₆ is selected from the group consisting of
(1) C₁₋₈alkyl,
(2) C₁₋₈alkoxy,
(3) C₁₋₈alkyl(halogen)₁₋₁₇,
(4) C₁₋₈alkoxy(halogen)₁₋₁₇,
(5) SO₂(C₁₋₈alkyl),
(6) cyano,
(7) halogen, and
(8) nitro.

Embodiments of compounds of Formula (Vb) include compounds wherein R₃ and R₆ are dependently selected from

| Cpd | R₃ | R₆ |
|---|---|---|
| 70 | 2-OCH₂CF₃ | 3,4-diOMe |
| 72 | 4-F-2-OCH(CH₃)₂ | 3,4-diOMe |
| 78 | 4-F-2-OCH(CH₃)₂ | 5-Cl-2-OCH₃ |
| 79 | 4-F-2-OCH(CH₃)₂ | 5-Cl-2-F |
| 80 | 4-F-2-OCH₂CF₃ | 3,4-diOMe |
| 81 | 4-F-2-OCH₂CF₃ | 5-Cl-2-OCH₃ |
| 82 | 4-F-2-OCH₂CF₃ | 5-Cl-2-F |
| 84 | 5-F-2-OCH₂CF₃ | 3,4-diOMe |

An embodiment of the invention is a compound of Formula (I) selected from the group consisting of 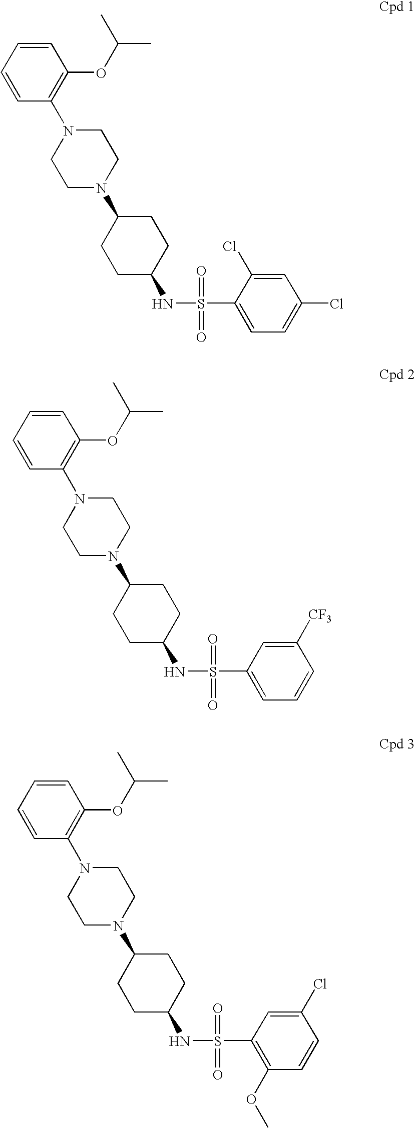 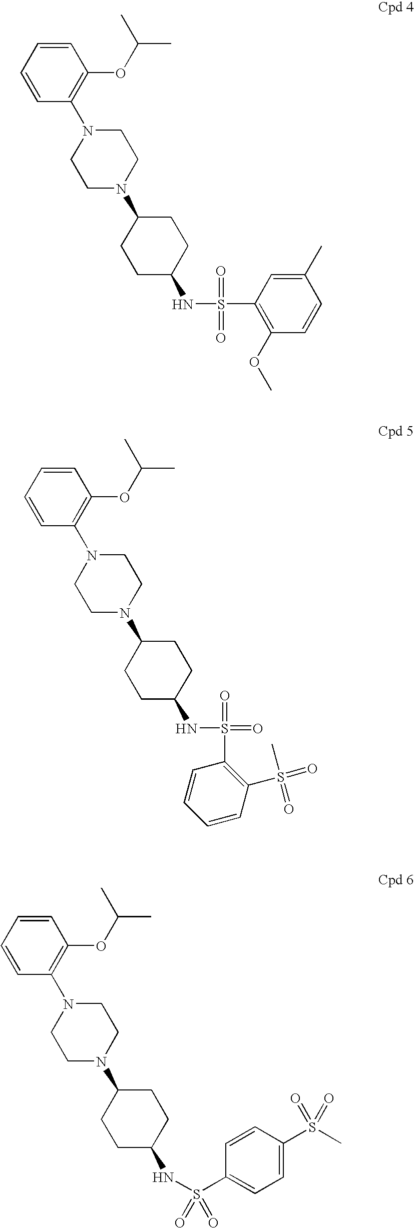 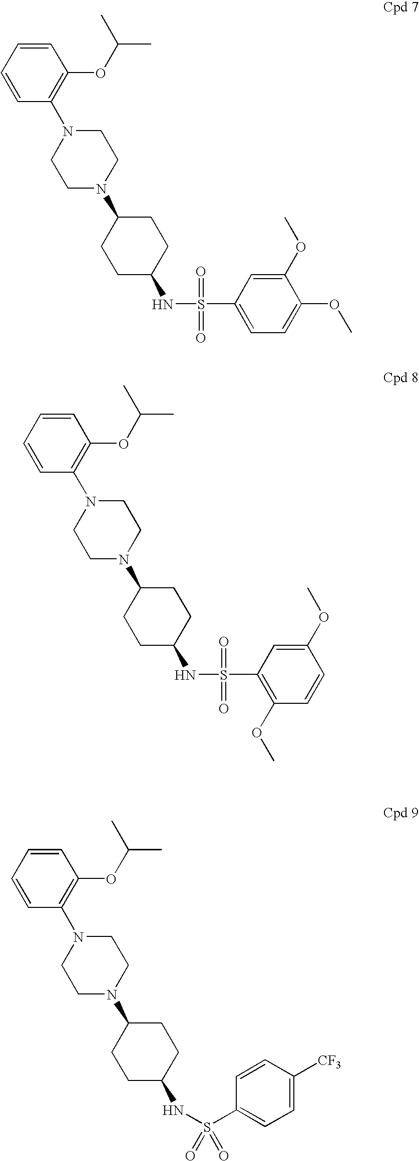 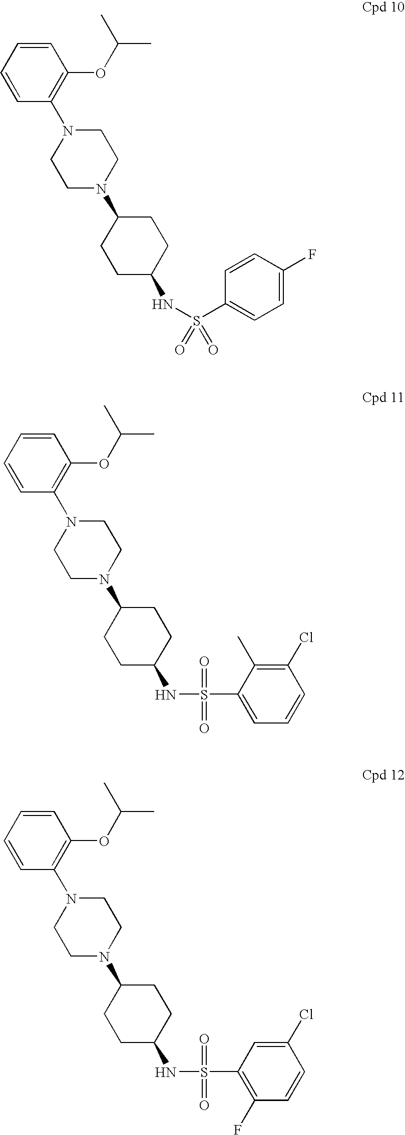 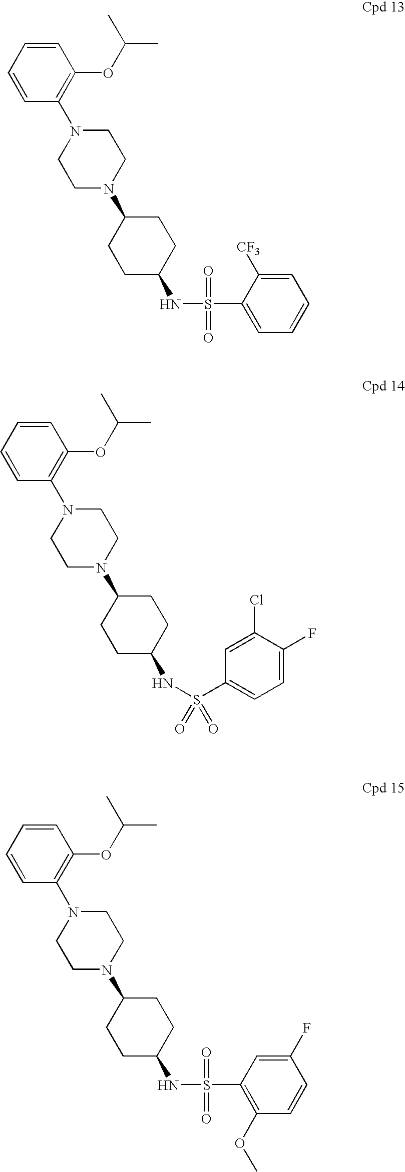 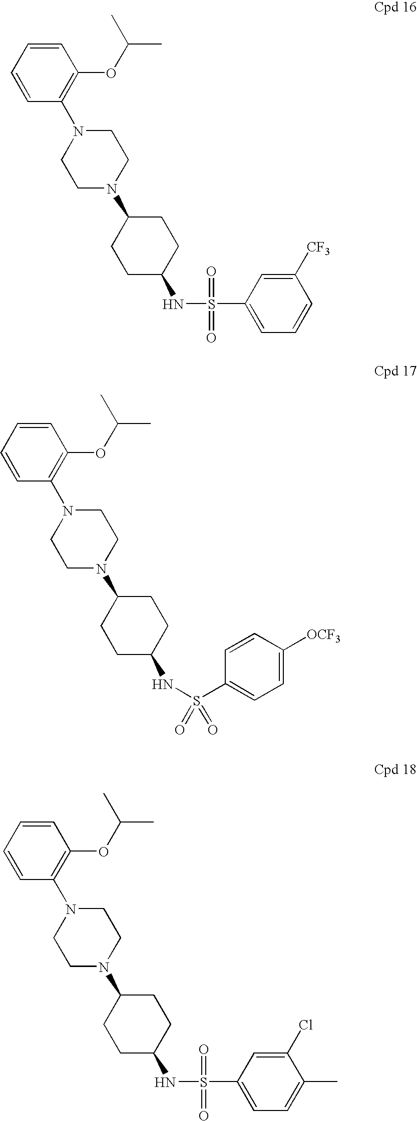 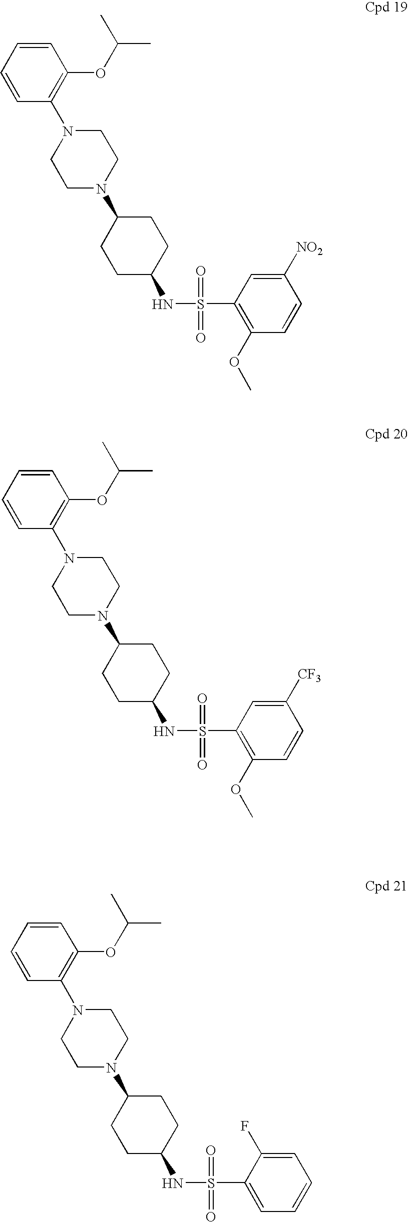 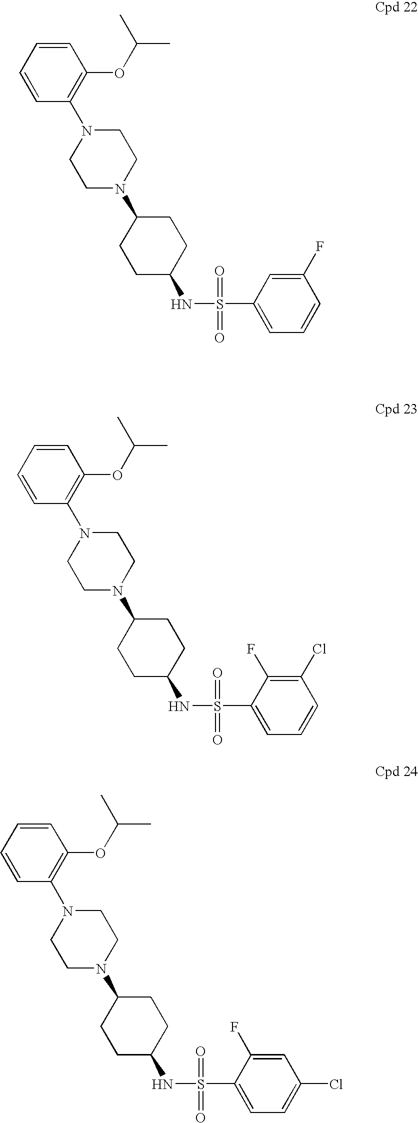 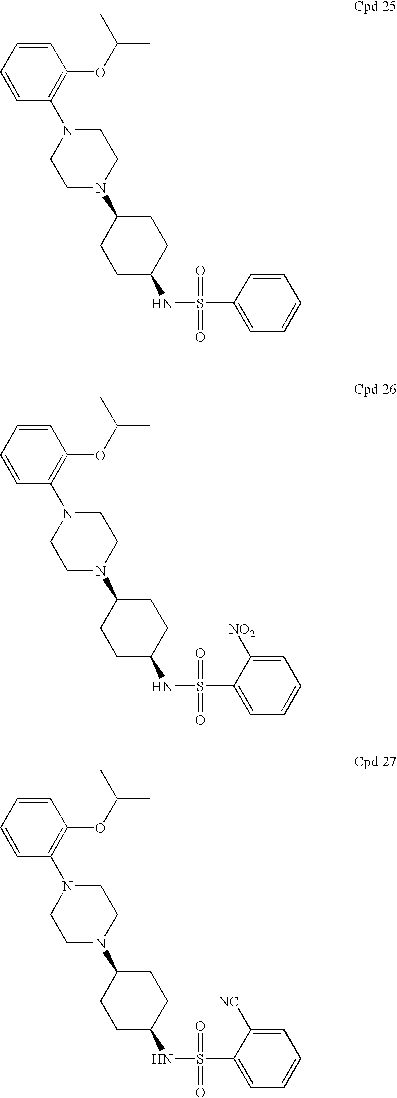 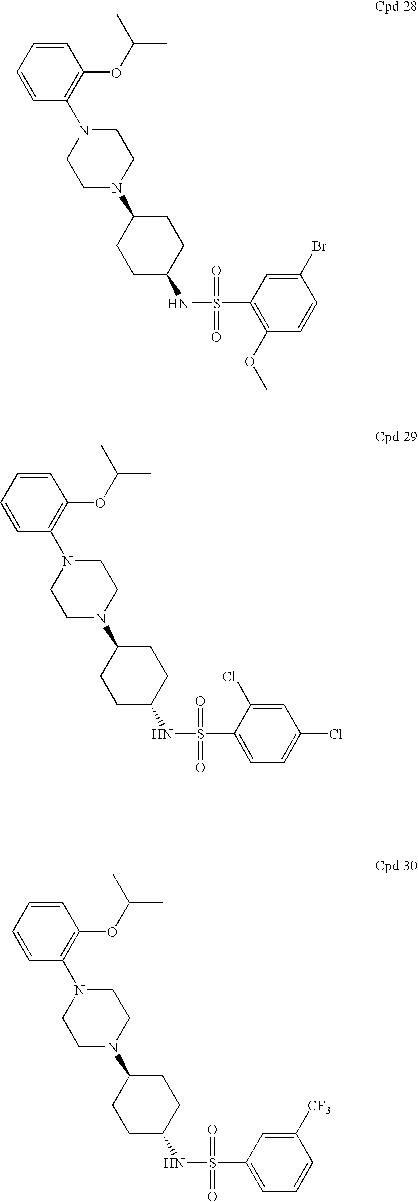 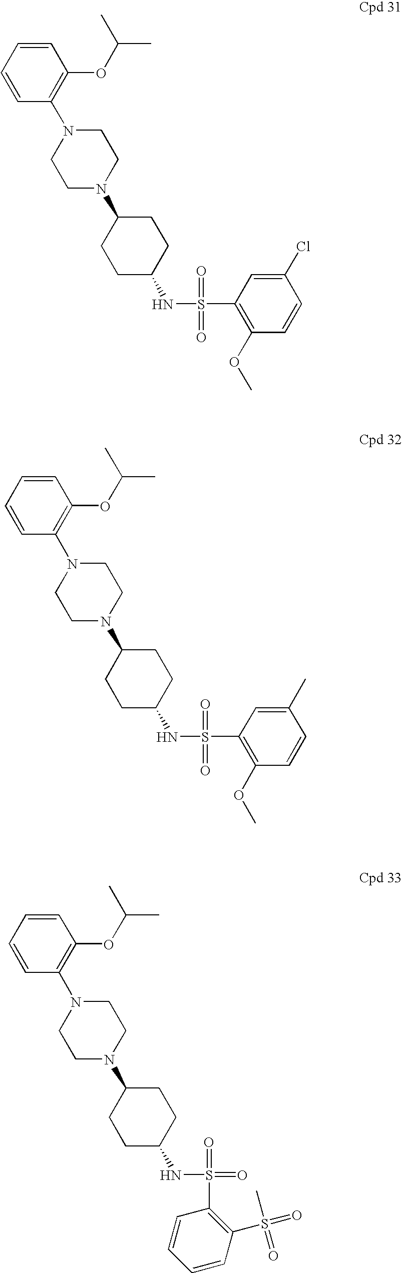 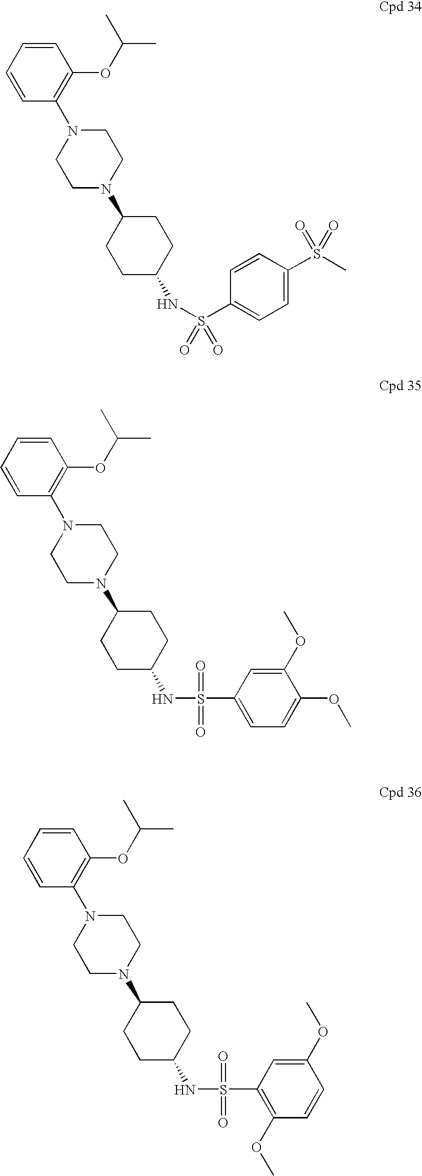 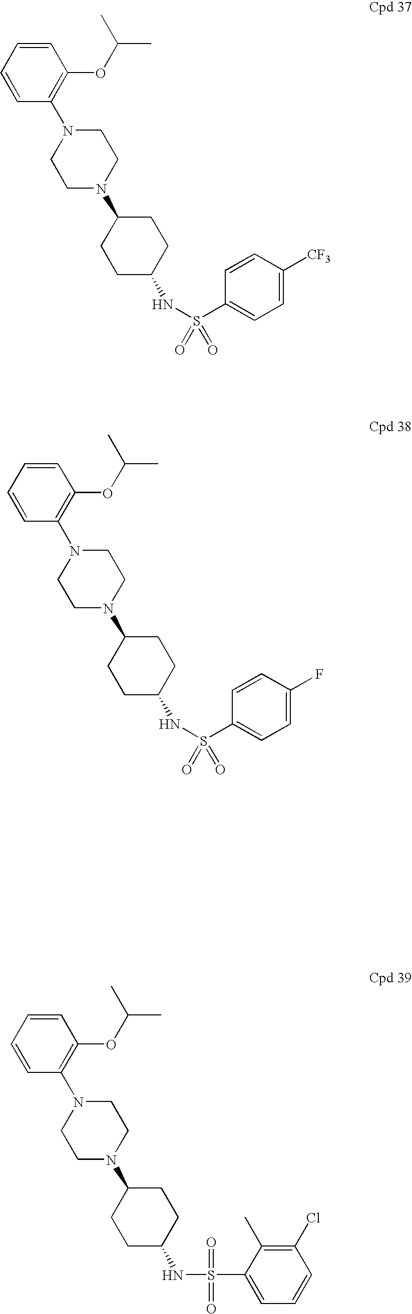 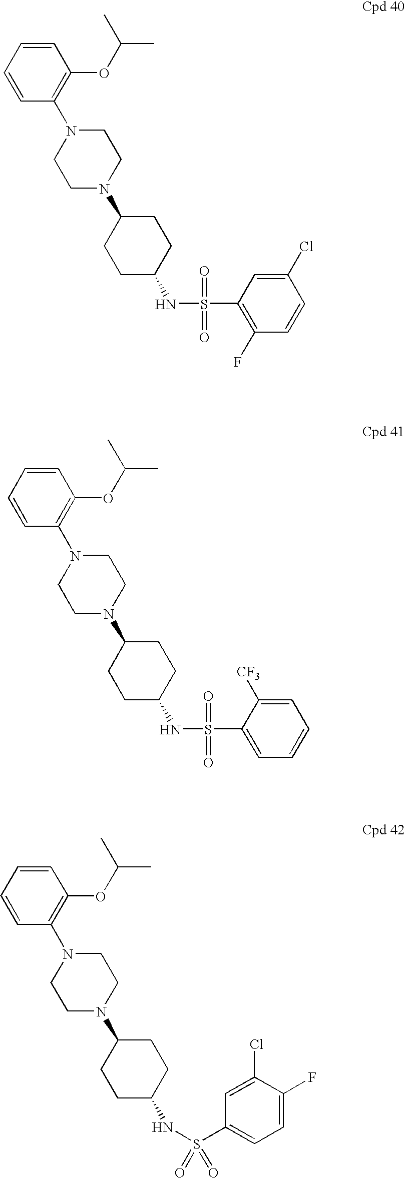 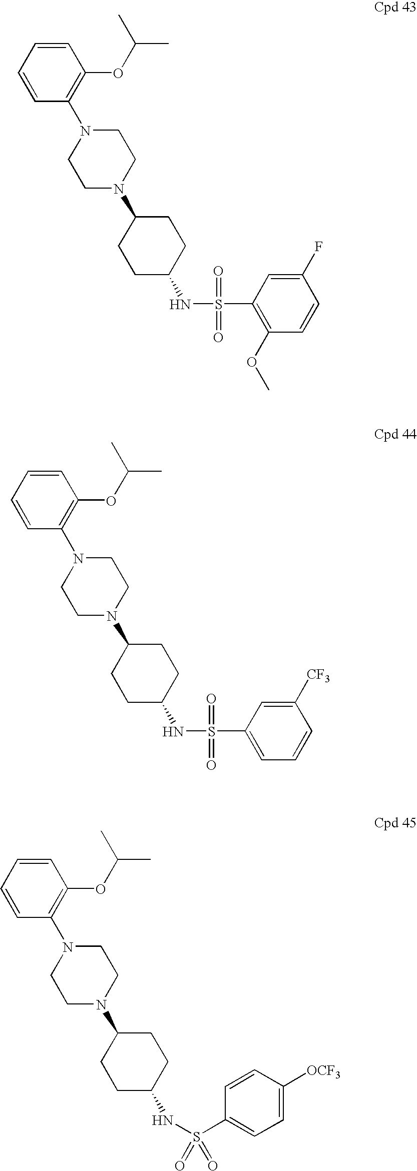 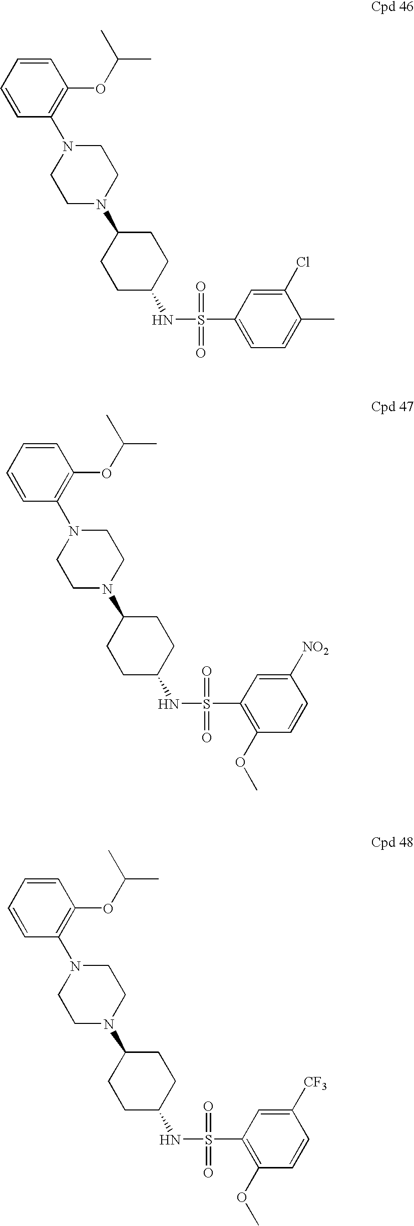 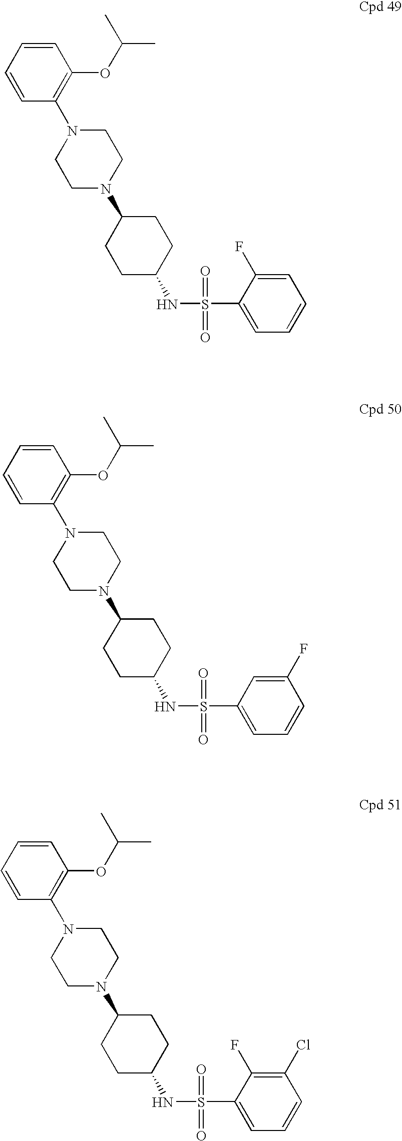 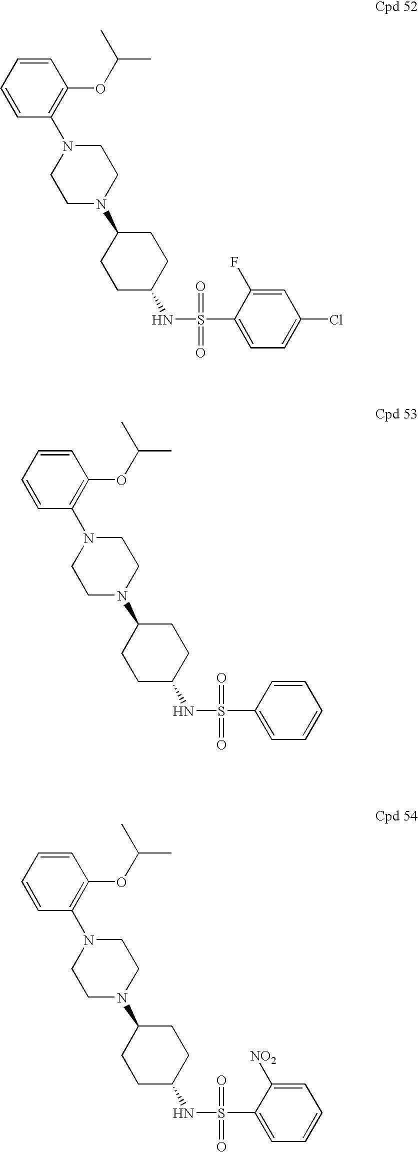 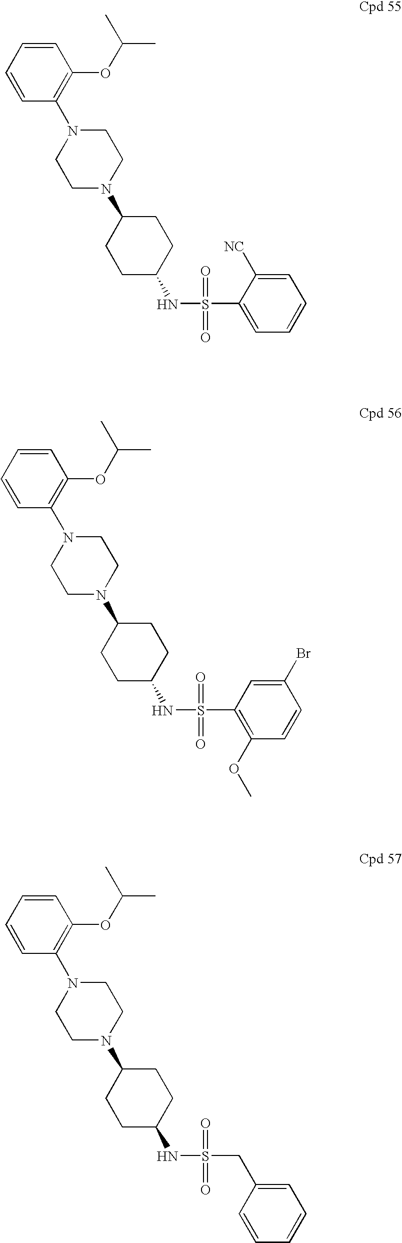 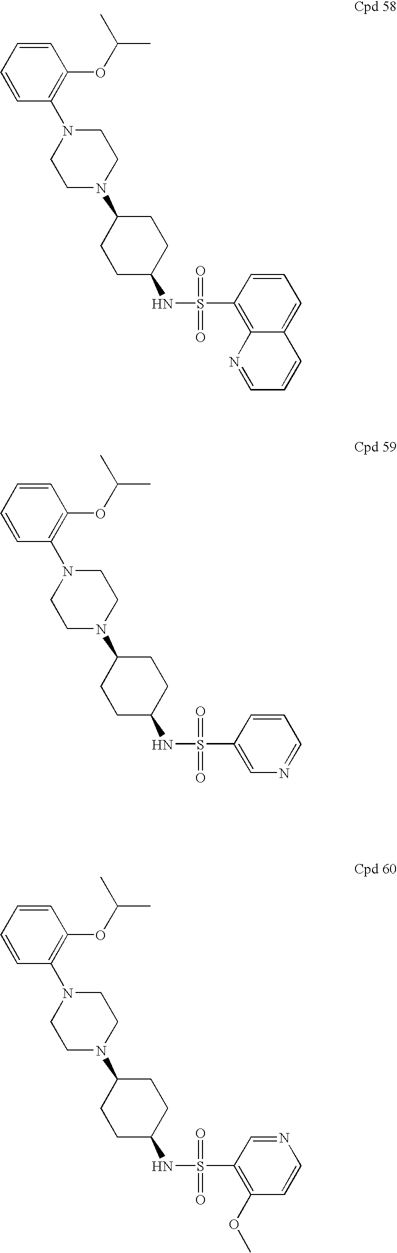 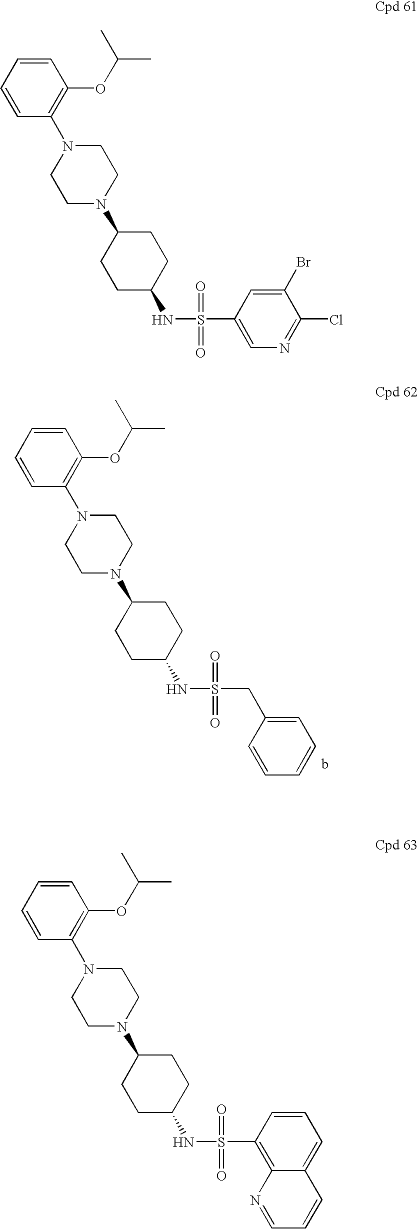 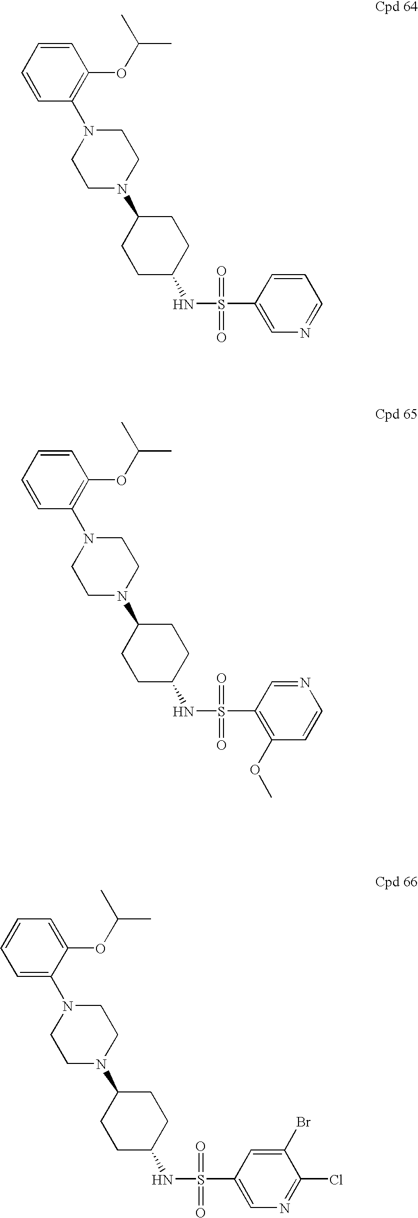 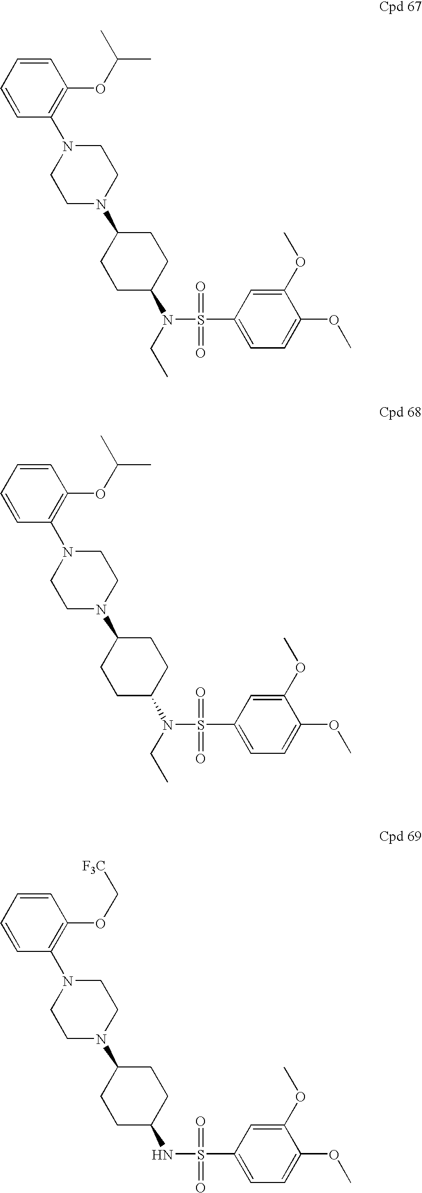 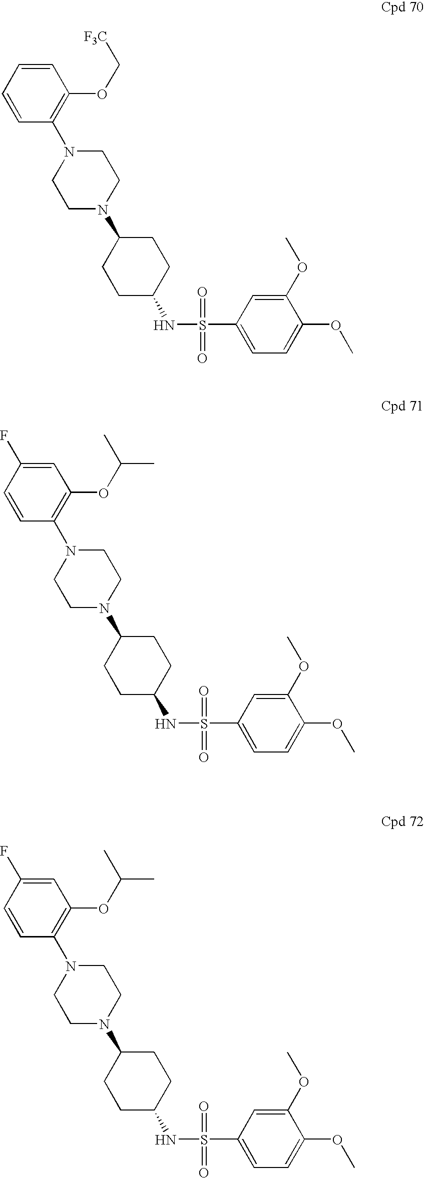 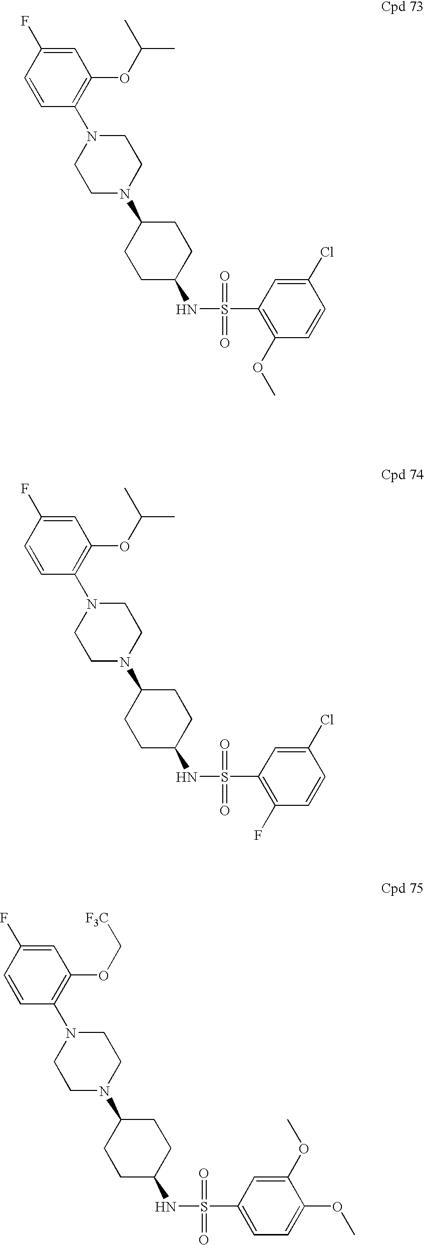 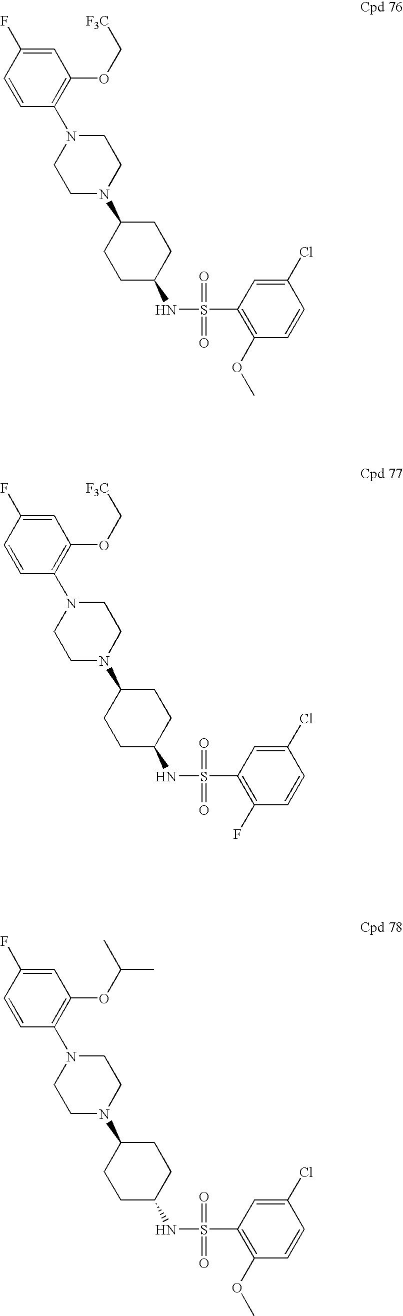 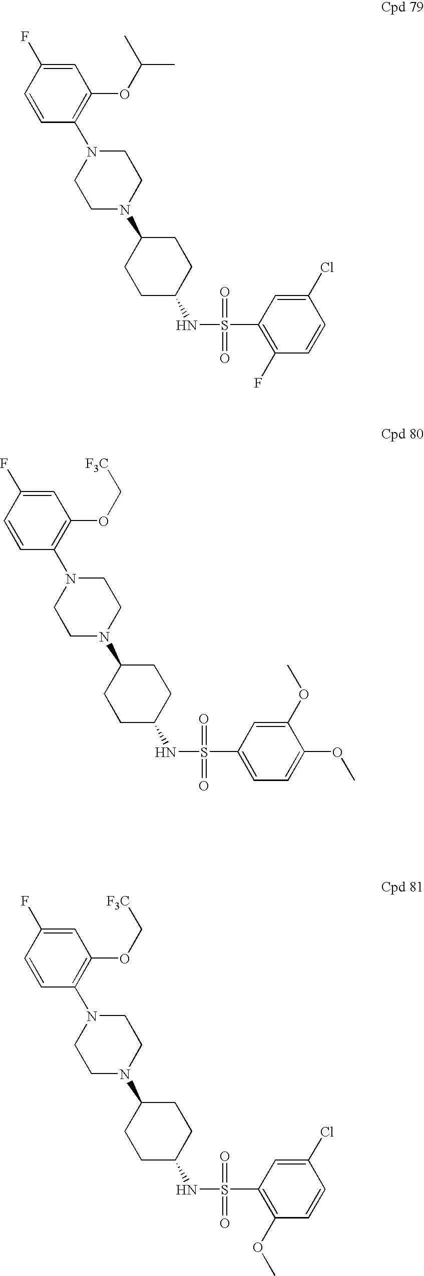 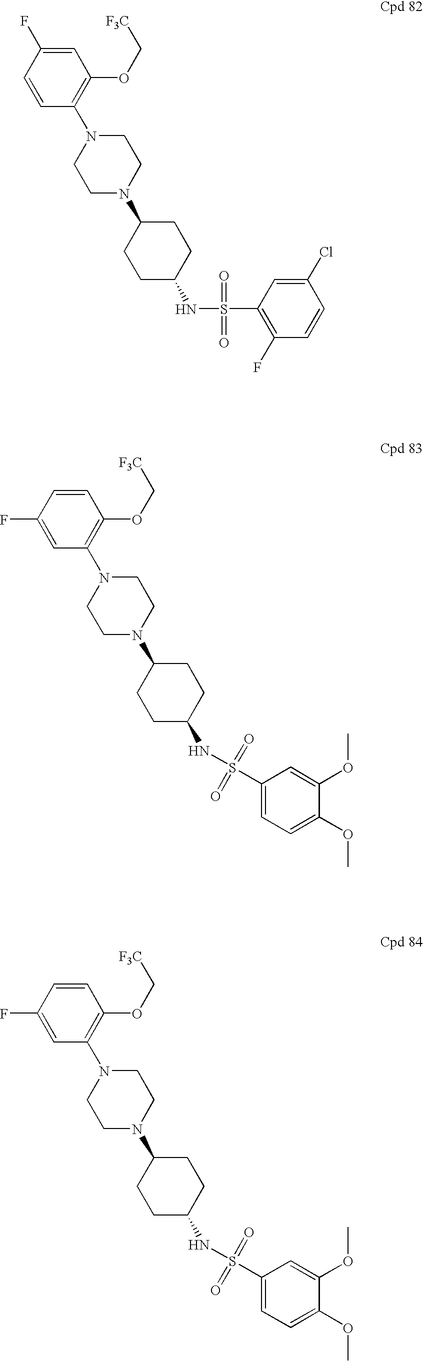
Compound Forms

The term “forms” and “forms thereof” means that the compounds of the present invention may exist in various salt, stereoisomer, crystalline, solvate, ester, prodrug or active metabolite forms and may be isolated according to methods known to those of ordinary skill in the art. The present invention encompasses all such compound forms, including active compounds in the form of essentially pure enantiomers, racemic mixtures and tautomers.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

The compounds of Formula I can be prepared as salts, in particular pharmaceutically acceptable salts.

Pharmaceutically acceptable acidic/anionic salts include the acetate, benzenesulfonate, benzoate, bicarbonate, bitartrate; bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide salts.

Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol, ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate, sodium hydroxide, triethanolamine or zinc.

Salts which are unsuitable for pharmaceutical uses but which can be employed, for example, for the isolation or purification of free compounds I or their pharmaceutically acceptable salts, are also included.

Certain compounds of the Formula (I) may exist in various stereoisomeric or tautomeric forms. The present invention encompasses all such dual α₁ₐ/α_{1d} adrenoceptor inhibiting compounds, including active compounds in the form of essentially pure enantiomers, racemic mixtures, pure geometric isomers (such as cis and trans stereoisomers), mixtures of geometric isomers, and tautomers.

The present invention indeed contemplates compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers, or enantiomers).

The term “stereoisomer” refers to isomers of identical constitution that differ in the arrangement of their atoms in space. Enantiomers are stereoisomers wherein an asymmetrically substituted carbon atom acts as a chiral center. The term “chiral” refers to a molecule that is not superposable on its mirror image, implying the absence of an axis and a plane or center of symmetry. The term “enantiomer” refers to one of a pair of molecular species that are mirror images of each other and are not superposable. The term “diastereomer” refers to stereoisomers that are not related as mirror images. The symbols “R” and “S” represent the configuration of substituents around a chiral carbon atom(s). The symbols “R*” and “S*” denote the relative configurations of of substituents around a chiral carbon atom(s). Where the compounds of the present application have at least one stereocenter, they accordingly exist as enantiomers. Where the compounds according to the present invention posses two or more stereocenters, they may additionally exist as diastereoisomers. It is to be understood that all such isomers and mixtures thereof are encompassed within the scope to the present invention.

The term “racemate” or “racemic mixture” refers to a compound of equimolar quantities of two enantiomeric species, wherein the compound is devoid of optical activity. The term “optical activity” refers to the degree to which a chiral molecule or nonracemic mixture of chiral molecules rotates the plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” (opposite sided) configuration, the substituents are on opposite sides in relationship to the carbon-carbon double bond; in the “Z” (same sided) configuration, the substituents are oriented on the same side in relationship to the carbon-carbon double bond. Substituent atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

The compounds of the present invention may be prepared as individual isomers by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using preparative TLC (thin layer chromatography) or a chiral HPLC column.

Furthermore, compounds of the present invention may have one or more polymorph or amorphous crystalline forms and as such are intended to be included in the scope of the invention. In addition, some of the compounds may form solvates with water (i.e., hydrates) or common organic solvents, and such are also intended to be encompassed within the scope of this invention.

Chemical Definitions

As used herein, the following terms are intended to have the following meanings (additional definitions are provided where needed throughout the Specification):

The term “C₁₋₈ alkyl,” whether used alone or as part of a substituent group, means a straight or branched chain monovalent hydrocarbon alkyl radical or alkyldiyl linking group comprising from 1 to 8 carbon atoms, wherein the radical is derived by the removal of one hydrogen atom from a single carbon atom and the alkyldiyl linking group is derived by the removal of one hydrogen atom from each of two carbon atoms in the chain, such as, for example methyl, ethyl, 1-propyl, 2-propyl, 1-butyl, 2-butyl, tertiary butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Examples include C₁₋₈alkyl, C₁₋₆alkyl and C₁₋₄alkyl groups.

The term “C₁₋₈alkyl(halogen)₁₋₁₇” means that 1 to 17 hydrogen atoms on a straight or branched chain monovalent hydrocarbon alkyl radical or alkyldiyl linking group are replaced by a halogen atom. For certain shorter alkyl chains, the maximum number of halogen atoms is limited; for example, if the alkyl only encompasses 1 carbon atom then the maximum number of halogen atoms is limited to 3, if the alkyl only encompasses 2 carbon atoms then the maximum number of halogen atoms is limited to 5 and so on.

The term “C₂₋₆alkenyl,” whether used alone or as part of a substituent group, means a straight or branched chain monovalent hydrocarbon alkyl or alkyldiyl radical radical having at least one carbon-carbon double bond, whereby the double bond is derived by the removal of one hydrogen atom from each of two adjacent carbon atoms of the alkyl radical. Atoms may be oriented about the double bond in either the cis (E) or trans (S) conformation. Typical alkenyl groups comprising from 2 to 6 carbon atoms, such as, for example, ethenyl, propenyl, allyl (2-propenyl), butenyl, pentenyl, hexenyl and the like. Examples include C₂₋₈alkenyl or C₂₋₄alkenyl groups.

The term “C₂₋₆alkynyl,” whether used alone or as part of a substituent group, means a straight or branched chain monovalent hydrocarbon alkyl or alkyldiyl radical radical having at least one carbon-carbon triple bond, whereby the triple-bond is derived by the removal of two hydrogen atoms from each of two adjacent carbon atoms of the alkyl radical. Typical alkynyl groups comprising from 2 to 6 carbon atoms, such as, for example, ethynyl, propynyl, butynyl, pentynyl, hexynyl and the like. Examples include C₂₋₈alkynyl or C₂₋₄alkynyl groups.

The term “alkoxy,” whether used alone or as part of a substituent group, refers to an alkyl or alkyldiyl radical attached through an oxygen linking atom, of the formula —O—C₁₋₈alkyl. For example, “C₁₋₄alkoxy” includes the radicals methoxy, ethoxy, propoxy, butoxy, and the like. In another example, the term “C₁₋₈ alkyloxy” means a straight or branched chain alkyloxy group comprising from 1 to 8 carbon atoms, such as, for example, methoxy, ethoxy, propoxy, butoxy, pentoxy, hexoxy, heptoxy, octoxy and the like. An alkoxy radical may be attached to a core molecule and further substituted where indicated. Examples include C₁₋₈alkoxy or C₁₋₄alkoxy groups.

The term “C₁₋₈alkoxy(halogen)₁₋₁₇” has the analogous meaning to “C₁₋₈alkyl(halogen)₁₋₁₇,” as defined above mutatis mutandis.

The term “C₁₋₈alkyl(hydroxy)₁₋₃” has the analogous meaning to “C₁₋₈alkyl(halogen)₁₋₁₇,” as defined above mutatis mutandis.

The term “cycloalkyl,” whether used alone or as part of a substituent group, refers to a saturated or partially unsaturated hydrocarbon ring system radical derived by the removal of one hydrogen atom from a single ring carbon atom. Typical cycloalkyl radicals include cyclopropyl, cyclobutyl, cyclopentyl, cyclohexyl, cycloheptyl or cyclooctyl, indanyl, indenyl, fluorenyl, adamantanyl and the like. Examples include C₃₋₈cycloalkyl, C₅₋₈cycloalkyl, C₃₋₁₂cycloalkyl or C₃₋₂₀cycloalkyl groups.

The term “heterocyclyl,” whether used alone or as part of a substituent group, refers to a saturated or partially unsaturated ring radical derived by the removal of one hydrogen atom from a single carbon or nitrogen ring atom.

Typical heterocyclyl radicals include 2H-pyrrole, 2-pyrrolinyl, or 3-pyrrolinyl), pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydro-furanyl, tetrahydro-thienyl, tetrahydro-pyranyl, tetrahydro-pyridazinyl, 1,3-benzodioxolyl or 2,3-dihydro-1,4-benzodioxinyl and the like.

The term “hetero” used as a prefix for a ring system refers to the replacement of at least one ring carbon atom with one or more heteroatoms independently selected from N, O, S, SO or SO₂. Examples include rings wherein 1, 2, 3 or 4 ring members are a nitrogen atom; or, 0, 1, 2 or 3 ring members are nitrogen atoms and 1 member is an oxygen or sulfur atom. When allowed by available valences, up to two adjacent ring members may be heteroatoms; wherein one heteroatom is nitrogen and the other is one heteroatom selected from N, S or O.

The term “aryl,” whether used alone or as part of a substituent group, refers to an aromatic cyclic hydrocarbon ring radical derived by the removal of one hydrogen atom from a single carbon atom of the ring system. Typical aryl radicals include phenyl, naphthalenyl, azulenyl, anthracenyl and the like.

The term “aromatic” refers to a cycloalkylic hydrocarbon ring system having an unsaturated, conjugated π electron system.

The term “heteroaryl,” whether used alone or as part of a substituent group, refers to a heteroaromatic cyclic hydrocarbon ring radical derived by the removal of one hydrogen atom from a single ring carbon atom of the ring system.

Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, azaindolyl, isoindolyl, benzo[b]furyl, benzo[b]thienyl, indazolyl, azaindazolyl, benzimidazolyl, benzthiazolyl, benzoxazolyl, benzisoxazolyl, benzothiadiazolyl, benzotriazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “halo” includes fluoro, chloro, bromo, and iodo.

The term “substituted” refers to a core molecule on which one or more hydrogen atoms have been replaced with one or more functional radical moieties. The number that is allowed by available valences limits the amount of substituents. Substitution is not limited to the core molecule, but may also occur on a substituent radical, whereby the substituent radical becomes a linking group.

The term “independently selected” refers to one or more substituents selected from a group of substituents variable group, wherein the selected substituents may be the same or different.

The term “dependently substituted” means that the structure variables are specified in an indicated combination.

In general, IUPAC nomenclature rules are used throughout this disclosure.

Methods of Use

The ability of compounds of the present invention to specifically bind to the α₁ₐ as well as to the α_{1d} receptor makes them useful for the treatment of BPH. The specificity of binding of compounds showing affinity for the α₁ₐ and the α_{1d} receptor is compared against the binding affinities to other types of alpha receptors.

An aspect of the present invention includes a compound of formula (I) having an IC₅₀ (50% inhibition concentration) against the activity of either or both the α₁ₐ and/or α_{1d} adrenoreceptor in a range of about 25 μM or less, of about 10 μM or less, of about 1 μM or less, of about 0.5 μM or less, of about 0.25 μM or less or of about 0.1 μM or less.

Another aspect of the present invention includes dual selective α₁ₐ/α_{1d} adrenoreceptor antagonists for treating, ameliorating or preventing a plurality of α₁ₐ and/or α_{1d} adrenoreceptor mediated disorders or diseases.

The usefulness of a compound of the present invention or composition thereof as a dual selective α₁ₐ/α_{1d} adrenoreceptor antagonist can be determined according to the methods disclosed herein. The scope of such use includes the treatment of benign prostatic hypertrophy and/or lower urinary tract symptoms.

An aspect of the use for a compound of formula (I) includes use of an instant compound as a marker, wherein the compound is labeled with a ligand such as a radioligand (selected from deuterium, tritium and the like).

The present invention is further directed to a method for treating, ameliorating or preventing an α₁ₐ and/or α_{1d} adrenoreceptor mediated disorder or disease in a subject in need of such treatment, amelioration or prevention comprising administering to the subject a therapeutically or prophylactically effective amount of a compound of formula (I) or a form or composition thereof.

An aspect of the method of the present invention further includes treating Benign Prostatic Hyperplasia in a subject in need of such treatment comprising administering to the subject in need of such treatment a therapeutically effective amount of a compound of formula (I) or a form or composition thereof.

An aspect of the method of the present invention further includes treating Lower Urinary Tract Symptoms in a subject in need of such treatment comprising administering to the subject in need of such treatment a therapeutically effective amount of a compound of formula (I) or a form or composition thereof.

Another aspect of the method of the present invention further includes administering to the subject an effective amount of a compound of formula (I) or composition thereof in the form of a medicament. Consequently, the invention encompasses the use of the compound of formula (I) as a medicament.

Accordingly, the present invention includes the use of a compound of formula (I) for the manufacture of a medicament for treating any of the diseases, disorders or conditions mentioned in any of the foregoing methods.

The term “subject” as used herein, refers to an animal, preferably a mammal, most preferably a human, which has been a patient or the object of treatment, prevention, observation or experiment.

The term “administering” is to be interpreted liberally in accordance with the methods of the present invention. Such methods include therapeutically or prophylactically administering an effective amount of a composition or medicament of the present invention at different times during the course of a therapy or concurrently in a combination form. Prophylactic administration can occur prior to the manifestation of symptoms characteristic of an α₁ₐ and/or α_{1d} adrenoreceptor mediated disorder or disease such that the disorder or disease is treated, ameliorated, prevented or otherwise delayed in its progression. The methods of the present invention are further to be understood as embracing all. therapeutic or prophylactic treatment regimens used by those skilled in the art.

The terms “therapeutically effective amount” or “prophylactically effective amount” refer to that amount of active compound or pharmaceutical agent that elicits the biological or medicinal response in a tissue system, animal or human, that is being sought by a researcher, veterinarian, medical doctor, or other clinician, which includes alleviation of the symptoms of the syndrome, disorder or disease being treated.

The effective amount of a compound of formula (I) exemplified in a method of the present invention is in a range of from about 0.001 mg/kg/day to about 300 mg/kg/day.

The term “medicament” refers to a product for use in treating, preventing or ameliorating a kinase mediated disease, disorder or condition.

Wherein the present invention is directed to the administration of a combination of a compound of Formula (I) and another agent for the treatment of BPH, the terms “therapeutically effective amount” or “prophylactically effective amount” shall mean that amount of the combination of agents taken together so that the combined effect elicits the desired biological or medicinal response.

Representative compounds of the present invention exhibit high selectivity for the α₁ₐ and α_{1d} adrenergic receptor. Moreover representative compounds of the present invention show low to very low affinity for the α_{1d} receptor. As a consequence hereof, the compounds of the present invention are beliefed to lower the intraurethral pressure without the unwanted side effects.

These compounds can be administered in dosages effective to antagonize the α₁ₐ and α_{1d} receptor where such treatment is needed, as in BHP.

Pharmaceutical Compositions

The present invention also has the objective of providing suitable topical, oral, systemic and parenteral pharmaceutical formulations for use in the novel methods of treatment of the present invention. The compositions containing compounds of this invention as the active ingredient for use in the specific antagonism of human α₁ₐ adrenergic receptors can be administered in a wide variety of therapeutic dosage forms in conventional vehicles for systemic administration.

The present invention also provides pharmaceutical compositions comprising one or more compounds of this invention in association with a pharmaceutically acceptable carrier. Preferably these compositions are in unit dosage forms such as tablets, pills, capsules, powders, graules, sterile parenteral solutions or suspensions, metered aerosol or liquid sprays, drops, ampoules, autoinjector devices or suppositories; for oral, parenteral, intranasal, sublingual or rectal administration, or for administration by inhalation or insulation.

Alternatively, the compositions may be presented in a form suitable for once-weeky or once-monthly administration; for example, an insoluble salt of the active compound, such as the decanoate salt, may be adapted to provide a depot preparation for intramuscular injection.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogenous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. An enteric layer can separate the two components. That enteric layer serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

As used herein, the term “composition” is intended to encompass a product comprising the specified ingredients in the specified amounts, as well as any product which results, directly or indirectly, from combination of the specified ingredients in the specified amounts.

An effective but non-toxic amount of the compound desired can be employed as a α₁ₐ/α_{1d} antagonistic agent. Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

Compounds of this invention may be administered in any of the foregoing compositions and according to dosage regimens established in the art whenever specific blockade of the human alphala adrenergic receptor is required.

The daily dosage of the products may be varied over a wide range from 0.001 to 3,000 mg per adult human per day. For oral administration, the compositions are preferably provided in the form of tablets containing 0.01, 0.05, 0.1, 0.5, 1.0, 2.5, 5.0, 10.0, 15.0, 25.0, 50.0 and milligrams of the active ingredient for the symptomatic adjustment of the dosage to the patient to be treated. A medicament typically contains from about 0.01 mg to about 500 mg of the active ingredient, preferably, from about 0.01 mg to about 100 3000 mg of active ingredient.

An effective amount of the drug is ordinarily supplied at a dosage level of from about 0.0002 mg/kg to about 20 mg/kg of body weight per day. Preferably, the range is from about 0.001 to 10 mg/kg of body weight per day, and especially from about 0.001 mg/kg to 7 mg/kg of body weight per day. The compounds may be administered on a regimen of 1 to 4 times per day.

Compounds of the present invention may be used alone at appropriate dosages defined by routine testing in order to obtain optimal antagonism of the human α₁ₐ/α_{1d} adrenergic receptor while minimizing any potential toxicity. In addition, co-administration or sequential administration of other agents which alleviate the effects of BPH is desirable.

Thus, in one embodiment, the method of the present invention includes administration of compounds of this invention and a human testosterone 5-α reductase inhibitor, including inhibitors of 5-α reductase isoenzyme 2.

The dosages of the α₁ₐ adrenergic receptor and testosterone 5-α reductase inhibitors are adjusted when combined to achieve desired effects. As those skilled in the art will appreciate, dosages of the 5-α reductase inhibitor and the α₁ₐ adrenergic receptor antagonist may be independently optimized and combined to achieve a synergistic result wherein the pathology is reduced more than it would be if either agent were used alone. In accordance with the method of the present invention, the individual components of the combination can be administered separately at different times during the course of therapy or concurrently in divided or single combination forms. The instant invention is therefore to be understood as embracing all such regimes of simultaneous or alternating treatment and the term “administering” is to be interpreted accordingly.

Thus, in one embodiment of the present invention, a method of treating BPH is provided which comprises administering to a subject in need of treatment any of the compounds of the present invention in combination with finasteride effective to treat BPH. The dosage of finasteride administered to the subject is about 0.01 mg per subject per day to about 50 mg per subject per day in combination with an α₁ₐ antagonist. Preferably, the dosage of finasteride in the combination is about 0.2 mg per subject per day to about 10 mg per subject per day, more preferably, about 1 to about 7 mg per subject to day, most preferably, about 5 mg per subject per day.

For the treatment of benign prostatic hyperplasia, compounds of this invention exhibiting α₁ₐ adrenergic receptor blockade can be combined with a therapeutically effective amount of a 5α-reductase isoenzyme 2 inhibitor, such as finasteride.

In other embodiments of the present inventions, a method of treating BPH is provided which comprises administering to a subject in need of treatment any of the compounds of the present invention in combination with a therapeutically effective amount of an anti-antiandrogenic agent, androgen receptor antagonists, selective androgen receptor modulators, urinary incontinence drugs (e.g. anti-muscarinics) or 5HT-receptor modulators.

In another embodiment of the present invention, a method of treating BPH is provided which comprises administering to a subject in need of treatment any of the compounds of the present invention in combination with a therapeutically effective amount of a PDE modulator.

Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art. No attempt has been made to optimize the yields obtained in any of the example reactions. One skilled in the art would know how to increase such yields through routine variations in reaction times, temperatures, solvents and/or reagents.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3rd Edition, John Wiley & Sons, 1999. The protecting groups may be removed at a convenient subsequent stage using methods known in the art.

Where the processes for the preparation of the compounds according to the invention give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

The terms used in describing the invention are commonly used and known to those skilled in the art. Some reagents are referred to as a chemical formula. Other reagents are referred to as abbreviations known to persons skilled in the art. When used herein, the following abbreviations have the indicated meanings:

| | |
|---|---|
| Boc | tert-butoxy carbonyl |
| CBz | benzyl carbonyl |
| Cpd | Compound |
| DCM | dichloromethane |
| DIC | 1,3-diisopropyl carbodiimide |
| DIBO | di-t-butyl oxalate |
| DMF | N,N-dimethyl formamide |
| EDCI | 1-(3-dimethylaminopropyl)-3- |
| | ethylcarbodiimide hydrochloride |
| Et | ethyl |
| EtOAc | ethylacetate |
| EtOH | ethanol |
| HOBt | 1-hydroxybenzotriazole hydrate |
| HX | general representation of an acid |
| LHMDS or LiHMDS | lithium hexamethyl disilazane or |
| | lithium bis(trimethylsilyl)amide |
| Me | methyl |
| MeOH | methanol |
| min/h/d/mp | minute/hour/day(s)/melting point |
| RT/rt/r.t. | room temperature |
| sat'd | saturated |
| TEA | triethylamine |
| TFA | trifluoroacetic acid |
| THF | tetrahydrofuran |
| TLC | thin layer chromatography |

All commercially available chemicals were obtained from commercial suppliers and used without further purification. Particular components or equipment used in the examples, such as reaction vessels and the like, are also commercially available.

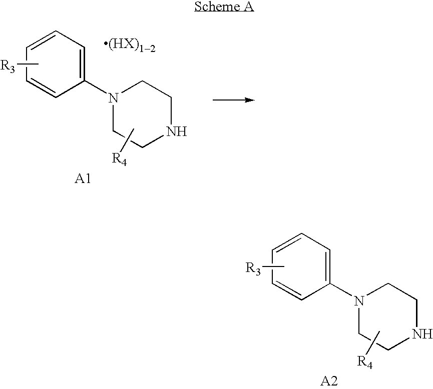

A substituted phenyl piperazine salt compound A1 was mixed with a solvent such as DCM and treated with a base such as 1N NaOH, then the two reaction layers were separated. The compound A1 salt is a mono or disalt form represented by (.HX)₁₋₂ which may be commercially available or synthesized using techniques known to one skilled in the art. The aqueous layer was extracted with a solvent such as DCM and the combined organic extracts were dried over K₂CO₃. The free base compound A2 was obtained by evaporating the solvent from the filtered solution on a rotary evaporator. The compound A2 free base may also be commercially available.

One or more of the R₃ or R₄ substituents for the compound A1 starting material may be amenable for further substitution using various reagent(s) and reaction conditions, thus enabling the preparation of other compounds that are representative of the invention by one skilled in the art.

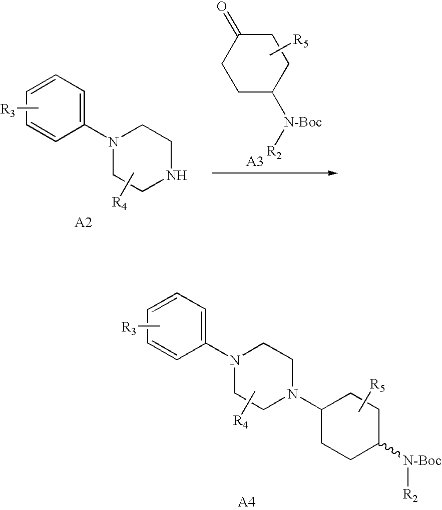

Compound A2, a substituted N-Boc-cyclohexanone compound A3, a reducing agent such as NaBH(OAc)₃ with or without a catalytic amount of acid such as HOAc and the like and a dry solvent such as anhydrous DCM were mixed together at rt to form a slurry and stirred under nitrogen atmosphere. The reaction was carried forward until the ketone compound A3 was no longer detected and then the mixture was diluted with a solvent such as DCM, washed with water or NH₄Cl (sat'd) and the like or a combination thereof and dried over Na₂SO₄. Compound A4 was obtained by evaporating the solvent from the filtered solution on a rotary evaporator and purifying the crude product by flash chromatography. Compound A4 was obtained as a mixture (represented by wave line bond) of cis and trans isomers.

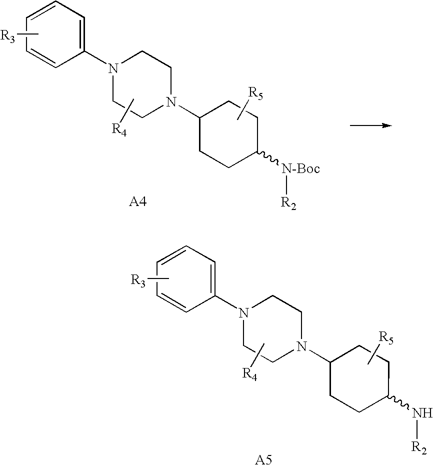

Compound A4 was dissolved with a solvent such as DCM at rt, then stirred into an acid such as TFA. The mixture was stirred for an additional 0.5 hr. The solvents were removed using a rotary evaporator and the residue was mixed with a solvent such as DCM, then made basic with a base such as 1N KOH to about pH 10. The aqueous layer was separated and extracted with a solvent such as DCM and the combined organic extracts were dried over K₂CO₃/Na₂SO₄ to provide compound A5 as a crude product which was used in the next step without further purification.

The R₂ substituent for the compound A3 or compound A4 reaction material may be further substituted either before or after deprotection using various reaction materials, reagent(s) and conditions, thus enabling the preparation of other compounds that are representative of the invention by one skilled in the art.

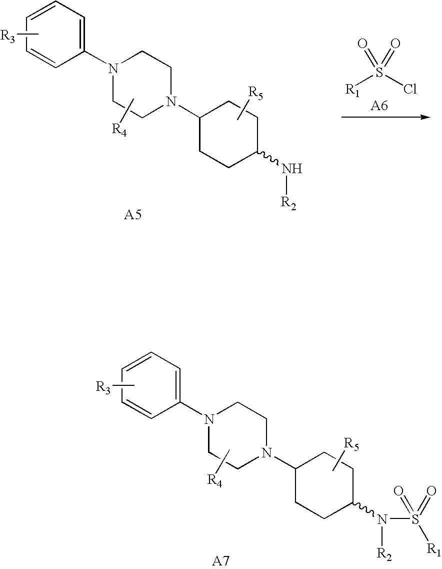

Compound A5 and an R₁ substituted sulfonyl chloride compound A6 were dissolved in a solvent such as DCM and a mild base such as K₂CO₃ was added, then the resulting turbid solution was stirred at rt. The reaction was carried forward until compound A5 was no longer detected and then the product solution containing the cis and trans mixture of compound A7 was filtered and separated (preferably on a preparative TLC plate using a solvent mixture such as 5% MeOH/DCM or using a SiO₂ column with an eluent solution such as 1-3% MeOH/CH₂Cl₂).

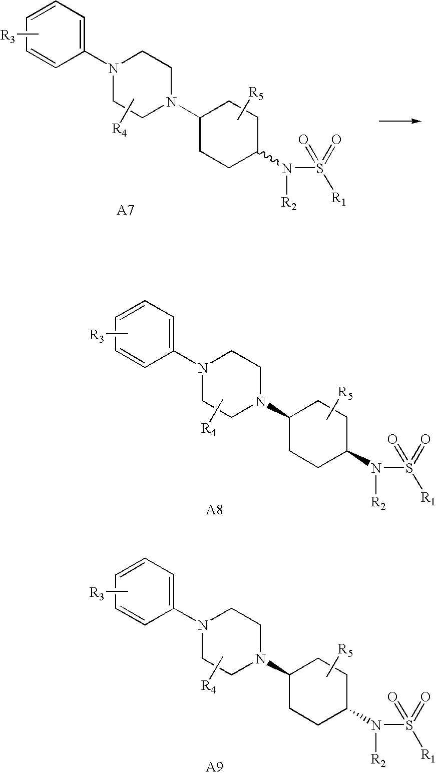

A cis isomer compound A8 (from the less polar spot when using TLC) and a trans isomer compound A9 (from the polar spot when using TLC) were isolated.

### EXAMPLE 1

### 2,4-Dichloro-N-cis-{4-[4-(2-isopropoxy-phenyl)-piperazine-1-yl]-cyclohexyl}-benzenesulfonamide (Cpd 1)

### 2,4-Dichloro-N-trans-{4-[4-(2-isopropoxy-phenyl)-piperazine-1-yl]-cyclohexyl}-benzenesulfonamide (Cpd 29)

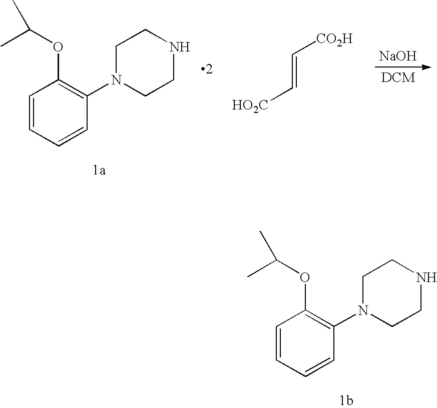

The 1-(2′-isoproxy-1-phenyl)piperazine difumarate salt compound 1a (10 g, 29.7 mmol) was mixed with DCM (100 mL) and treated with 1N NaOH (80 mL) whereupon the two layers were separated. The aqueous layer was extracted with DCM (3×20 mL) and the combined organic extracts were dried over K₂CO₃. The free base compound 1b (6.5 g) was obtained from evaporating the solvent of the filtered dry solution on a rotary evaporator.

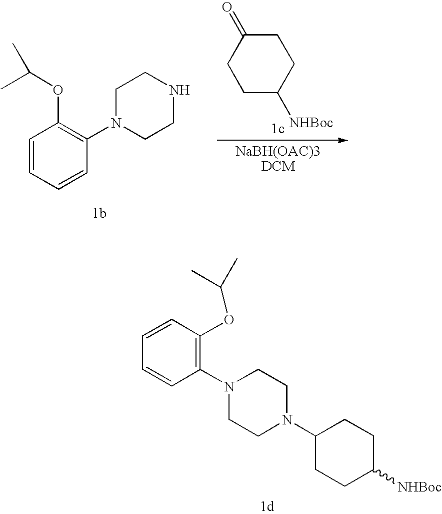

1-(2′-isoproxy-1-phenyl)piperazine compound 1b (3.00 g, 13.6 mmol), N-Boc-4-amino-cyclohexanone compound 1c (2.90 g, 13.6 mmol), NaBH(OAc)₃ (8.6 g, 40.8 mmol), HOAc (1 mL) and anhydrous DCM (80 mL) were mixed together at room temperature to form a white slurry and stirred under nitrogen atmosphere. The reaction was carried forward until a yellowish solution was formed and no ketone was detected by TLC (100% AcOEt, 18 hrs). The reaction mixture was diluted with DCM (80 mL), washed with H₂O, NH₄Cl (sat'd) and dried over Na₂SO₄. The crude product was obtained by removing solvent on a rotary evaporator from the filtered dry solution. Compound 1d (5.43 g, 13.02 mmol, yield 96%) was obtained by flash chromatography (100% AcOEt, silica gel) as a white sticky oil. LC-MS at 2.85 minutes, m/z 418.2 (MH⁺). ¹H NMR (CDCl₃, TMS) δ 1.38 (d, J=6.0 Hz, 6H), 1.46 (s, 9H), 1.50-2.40 (m, 8H), 2.74 (br s, 4H), 3.13 (br s, 4H), 3.20-4.400 (m, 2H), 4.20-4.90 (m, 2H), 6.80-7.05 (m, 4H).

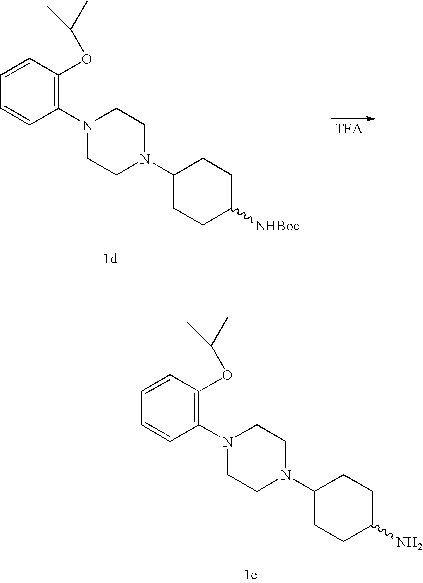

Compound 1d (5.43 g, 13.0 mmol) was dissolved into DCM (25 mL, yellowish clear solution) at rt. The solution was stirred with TFA (10 mL) for 0.5 hr. The volatiles were removed on a rotary evaporator, the yellow residue was mixed with DCM (80 mL), then made basic with 1 N KOH to pH 10. The aqueous layer was separated, then extracted with DCM (3×20 mL). The combined organic extracts were dried over K₂CO₃/Na₂SO₄ and the crude product compound 1e (3.08 g, yield 74.6%) was obtained as a white sticky oil and was used directly without further purification. LC-MS at 2.258 minutes, m/z 318.2 (MH⁺). ¹H NMR (CDCl₃, TMS) δ 1.05-1.20 (m, 1H), 1.20-1.45 (m, 3H), 1.30 (d, J=6.0 Hz, 6H), 1.48-1.76 (m, 4H), 1.83-2.02 (m, 2H), 2.20-2.50 (m, 1H), 2.55-2.85 (m, 4H), 2.95-3.25 (m, 5H), 4.54-4.60 (m, 1H), 6.80-6.92 (m, 4H).

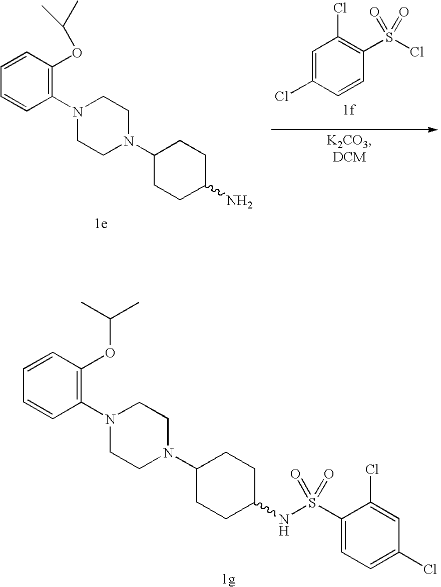

Compound 1e (0.050 g, 0.16 mmol) and 2,4-dichlorobenzenesulfonyl chloride compound 1f (0.059 g, 0.24 mmol) were dissolved in DCM (2 mL), then K₂CO₃ (0.10 g) was added. The yellowish turbid solution was stirred at rt and monitored by TLC (5% MeOH/DCM) and LC-MS. When the reaction was complete (compound 1e was not detected), the product solution containing compound 1g was filtered and loaded on a preparative TLC plate. The plate was developed using a solvent mixture (5% MeOH/DCM) to separate the isomeric mixture into compound 1 and compound 29.

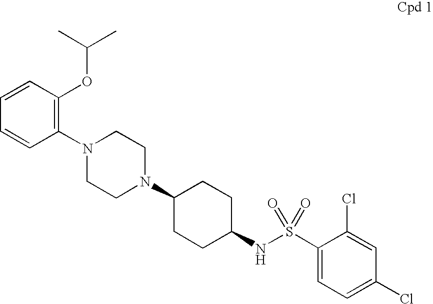

Compound 1 (0.0337 g) was isolated as a yellowish oil from the less polar spot and was assigned as the cis isomer. LC-MS at 3.132 min., m/z 526.2 (MH⁺). ¹H NMR (CDCl₃, TMS) δ 1.37 (d, J=6.3 Hz, 6H), 1.42-2.15 (m, 8H), 2.15-2.35 (m, 1H), 2.60-2.85 (m, 4H), 3.00-3.25 (m, 4H), 3.30-3.50 (m, 1H), 4.50-4.80 (m, 1H), 5.20-5.60 (m, 1H), 6.80-7.40 (m, 4H), 7.28 (s, 1 H), 7.42 (dd, J=2.1 Hz, J₂=8.5 Hz, 1H), 7.57 (d, J=2.1 Hz, 1H).

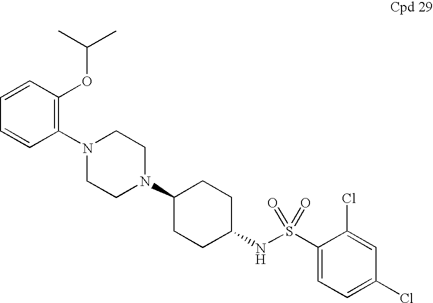

Compound 29 was isolated as a yellowish oil, (0.0131 g) from the polar spot and was assigned as the trans isomer. LC-MS at 3.081 minutes, m/z 526.2 (100, M⁺). ¹H NMR (CDCl₃, TMS) δ 1.37 (d, J=6.6 Hz, 6H), 1.50-2.15 (m, 9H), 2.15-2.40 (m, 1H), 2.55-2.80 (m, 4H), 2.95-3.20 (m, 4H), 4.50-4.68 (m, 1H), 4.80-4.95 (m, 1H), 6.80-7.03 (m, 4H), 7.28 (s, 1H), 7.43 (dd, J₁=2.1 Hz, J₂=8.5 Hz, 1H), 7.56 (d, J=2.1 Hz, 1H).

Using the procedure of Example 1, other compounds that are representative of the invention may be prepared by varying the starting materials, reagent(s) and conditions used (MS represents m/z of M⁺ or MH⁺):

| Cpd | Name | MS |
|---|---|---|
| 2 | N-cis-{4-[4-(2-Isopropoxy-phenyl)-piperazin-1-yl]-cyclohexyl}-3- | 526 |
| | trifluoromethyl-benzenesulfonamide | |
| 3 | 5-Chloro-N-cis-{4-[4-(2-isopropoxy-phenyl)-piperazin-1-yl]- | 522 |
| | cyclohexyl}-2-methoxy-benzenesulfonamide | |
| 4 | N-cis-{4-[4-(2-Isopropoxy-phenyl)-piperazin-1-yl]-cyclohexyl}-2- | 502 |
| | methoxy-5-methyl-benzenesulfonamide | |
| 5 | N-cis-{4-[4-(2-Isopropoxy-phenyl)-piperazin-1-yl]-cyclohexyl}-2- | 536 |
| | methanesulfonyl-benzenesulfonamide | |
| 6 | N-cis-{4-[4-(2-Isopropoxy-phenyl)-piperazin-1-yl]-cyclohexyl}-4- | 536 |
| | methanesulfonyl-benzenesulfonamide | |
| 7 | N-cis-{4-[4-(2-Isopropoxy-phenyl)-piperazin-1-yl]-cyclohexyl}-3,4- | 518 |
| | dimethoxy-benzenesulfonamide | |
| 8 | N-cis-{4-[4-(2-Isopropoxy-phenyl)-piperazin-1-yl]-cyclohexyl}-2,5- | 518 |
| | dimethoxy-benzenesulfonamide | |
| 9 | N-cis-{4-[4-(2-Isopropoxy-phenyl)-piperazin-1-yl]-cyclohexyl}-4- | 526 |
| | trifluoromethyl-benzenesulfonamide | |
| 10 | 4-Fluoro-N-cis-{4-[4-(2-isopropoxy-phenyl)-piperazin-1-yl]- | 476 |
| | cyclohexyl}-benzenesulfonamide | |
| 11 | 3-Chloro-N-cis-{4-[4-(2-isopropoxy-phenyl)-piperazin-1-yl]- | 506 |
| | cyclohexyl}-2-methyl-benzenesulfonamide | |
| 12 | 5-Chloro-2-fluoro-N-cis-{4-[4-(2-isopropoxy-phenyl)-piperazin-1-yl]- | 510 |
| | cyclohexyl}-benzenesulfonamide | |
| 13 | N-cis-{4-[4-(2-Isopropoxy-phenyl)-piperazin-1-yl]-cyclohexyl}-2- | 526 |
| | trifluoromethyl-benzenesulfonamide | |
| 14 | 3-Chloro-4-fluoro-N-cis-{4-[4-(2-isopropoxy-phenyl)-piperazin-1-yl]- | 510 |
| | cyclohexyl}-benzenesulfonamide | |
| 15 | 5-Fluoro-N-cis-{4-[4-(2-isopropoxy-phenyl)-piperazin-1-yl]- | 505 |
| | cyclohexyl}-2-methoxy-benzenesulfonamide | |
| 16 | N-cis-{4-[4-(2-Isopropoxy-phenyl)-piperazin-1-yl]-cyclohexyl}-3- | 525 |
| | trifluoromethyl-benzenesulfonamide | |
| 17 | N-cis-{4-[4-(2-Isopropoxy-phenyl)-piperazin-1-yl]-cyclohexyl}-4- | 541 |
| | trifluoromethoxy-benzenesulfonamide | |
| 18 | 3-Chloro-N-cis-{4-[4-(2-isopropoxy-phenyl)-piperazin-1-yl]- | 506 |
| | cyclohexyl}-4-methyl-benzenesulfonamide | |
| 19 | N-cis-{4-[4-(2-Isopropoxy-phenyl)-piperazin-1-yl]-cyclohexyl}-2- | 532 |
| | methoxy-5-nitro-benzenesulfonamide | |
| 20 | N-cis-{4-[4-(2-Isopropoxy-phenyl)-piperazin-1-yl]-cyclohexyl}-2- | 555 |
| | methoxy-5-trifluoromethyl-benzenesulfonamide | |
| 21 | 2-Fluoro-N-cis-{4-[4-(2-isopropoxy-phenyl)-piperazin-1-yl]- | 475 |
| | cyclohexyl}-benzenesulfonamide | |
| 22 | 3-Fluoro-N-cis-{4-[4-(2-isopropoxy-phenyl)-piperazin-1-yl]- | 475 |
| | cyclohexyl}-benzenesulfonamide | |
| 23 | 3-Chloro-2-fluoro-N-cis-{4-[4-(2-isopropoxy-phenyl)-piperazin-1-yl]- | 510 |
| | cyclohexyl}-benzenesulfonamide | |
| 24 | 4-Chloro-2-fluoro-N-cis-{4-[4-(2-isopropoxy-phenyl)-piperazin-1-yl]- | 510 |
| | cyclohexyl}-benzenesulfonamide | |
| 25 | N-cis-{4-[4-(2-Isopropoxy-phenyl)-piperazin-1-yl]-cyclohexyl}- | 457 |
| | benzenesulfonamide | |
| 26 | N-cis-{4-[4-(2-Isopropoxy-phenyl)-piperazin-1-yl]-cyclohexyl}-2- | 502 |
| | nitro-benzenesulfonamide | |
| 27 | 2-Cyano-N-cis-{4-[4-(2-isopropoxy-phenyl)-piperazin-1-yl]- | 482 |
| | cyclohexyl}-benzenesulfonamide | |
| 28 | 5-Bromo-N-cis-{4-[4-(2-isopropoxy-phenyl)-piperazin-1-yl]- | 566 |
| | cyclohexyl}-2-methoxy-benzenesulfonamide | |
| 30 | N-trans-{4-[4-(2-Isopropoxy-phenyl)-piperazin-1-yl]-cyclohexyl}-3- | 526 |
| | trifluoromethyl-benzenesulfonamide | |
| 31 | 5-Chloro-N-trans-{4-[4-(2-isopropoxy-phenyl)-piperazin-1-yl]- | 522 |
| | cyclohexyl}-2-methoxy-benzenesulfonamide | |
| 32 | N-trans-{4-[4-(2-Isopropoxy-phenyl)-piperazin-1-yl]-cyclohexyl}-2- | 502 |
| | methoxy-5-methyl-benzenesulfonamide | |
| 33 | N-trans-{4-[4-(2-Isopropoxy-phenyl)-piperazin-1-yl]-cyclohexyl}-2- | 536 |
| | methanesulfonyl-benzenesulfonamide | |
| 34 | N-trans-{4-[4-(2-Isopropoxy-phenyl)-piperazin-1-yl]-cyclohexyl}-4- | 536 |
| | methanesulfonyl-benzenesulfonamide | |
| 35 | N-trans-{4-[4-(2-Isopropoxy-phenyl)-piperazin-1-yl]-cyclohexyl}-3,4- | 518 |
| | dimethoxy-benzenesulfonamide | |
| 36 | N-trans-{4-[4-(2-Isopropoxy-phenyl)-piperazin-1-yl]-cyclohexyl}-2,5- | 518 |
| | dimethoxy-benzenesulfonamide | |
| 37 | N-trans-{4-[4-(2-Isopropoxy-phenyl)-piperazin-1-yl]-cyclohexyl}-4- | 526 |
| | trifluoromethyl-benzenesulfonamide | |
| 38 | 4-Fluoro-N-trans-{4-[4-(2-isopropoxy-phenyl)-piperazin-1-yl]- | 476 |
| | cyclohexyl}-benzenesulfonamide | |
| 39 | 3-Chloro-N-trans-{4-[4-(2-isopropoxy-phenyl)-piperazin-1-yl]- | 506 |
| | cyclohexyl}-2-methyl-benzenesulfonamide | |
| 40 | 5-Chloro-2-fluoro-N-trans-{4-[4-(2-isopropoxy-phenyl)-piperazin-1- | 510 |
| | yl]-cyclohexyl}-benzenesulfonamide | |
| 41 | N-trans-{4-[4-(2-Isopropoxy-phenyl)-piperazin-1-yl]-cyclohexyl}-2- | 526 |
| | trifluoromethyl-benzenesulfonamide | |
| 42 | 3-Chloro-4-fluoro-N-trans-{4-[4-(2-isopropoxy-phenyl)-piperazin-1- | 510 |
| | yl]-cyclohexyl}-benzenesulfonamide | |
| 43 | 5-Fluoro-N-trans-{4-[4-(2-isopropoxy-phenyl)-piperazin-1-yl]- | 505 |
| | cyclohexyl}-2-methoxy-benzenesulfonamide | |
| 44 | N-trans-{4-[4-(2-Isopropoxy-phenyl)-piperazin-1-yl]-cyclohexyl}-3- | 525 |
| | trifluoromethyl-benzenesulfonamide | |
| 45 | N-trans-{4-[4-(2-Isopropoxy-phenyl)-piperazin-1-yl]-cyclohexyl}-4- | 541 |
| | trifluoromethoxy-benzenesulfonamide | |
| 46 | 3-Chloro-N-trans-{4-[4-(2-isopropoxy-phenyl)-piperazin-1-yl]- | 506 |
| | cyclohexyl}-4-methyl-benzenesulfonamide | |
| 47 | N-trans-{4-[4-(2-Isopropoxy-phenyl)-piperazin-1-yl]-cyclohexyl}-2- | 532 |
| | methoxy-5-nitro-benzenesulfonamide | |
| 48 | N-trans-{4-[4-(2-Isopropoxy-phenyl)-piperazin-1-yl]-cyclohexyl}-2- | 555 |
| | methoxy-5-trifluoromethyl-benzenesulfonamide | |
| 49 | 2-Fluoro-N-trans-{4-[4-(2-isopropoxy-phenyl)-piperazin-1-yl]- | 475 |
| | cyclohexyl}-benzenesulfonamide | |
| 50 | 3-Fluoro-N-trans-{4-[4-(2-isopropoxy-phenyl)-piperazin-1-yl]- | 475 |
| | cyclohexyl}-benzenesulfonamide | |
| 51 | 3-Chloro-2-fluoro-N-trans-{4-[4-(2-isopropoxy-phenyl)-piperazin-1- | 510 |
| | yl]-cyclohexyl}-benzenesulfonamide | |
| 52 | 4-Chloro-2-fluoro-N-trans-{4-[4-(2-isopropoxy-phenyl)-piperazin-1- | 510 |
| | yl]-cyclohexyl}-benzenesulfonamide | |
| 53 | N-trans-{4-[4-(2-Isopropoxy-phenyl)-piperazin-1-yl]-cyclohexyl}- | 457 |
| | benzenesulfonamide | |
| 54 | N-trans-{4-[4-(2-Isopropoxy-phenyl)-piperazin-1-yl]-cyclohexyl}-2- | 502 |
| | nitro-benzenesulfonamide | |
| 55 | 2-Cyano-N-trans-{4-[4-(2-isopropoxy-phenyl)-piperazin-1-yl]- | 482 |
| | cyclohexyl}-benzenesulfonamide | |
| 56 | 5-Bromo-N-trans-{4-[4-(2-isopropoxy-phenyl)-piperazin-1-yl]- | 566 |
| | cyclohexyl}-2-methoxy-benzenesulfonamide | |
| 57 | N-cis-{4-[4-(2-Isopropoxy-phenyl)-piperazin-1-yl]-cyclohexyl}-C- | 471 |
| | phenyl-methanesulfonamide | |
| 58 | Quinoline-8-sulfonic acid N-cis-{4-[4-(2-isopropoxy-phenyl)- | 508 |
| | piperazin-1-yl]-cyclohexyl}-amide | |
| 59 | Pyridine-3-sulfonic acid N-cis-{4-[4-(2-isopropoxy-phenyl)-piperazin- | 458 |
| | 1-yl]-cyclohexyl}-amide | |
| 60 | 4-Methoxy-pyridine-3-sulfonic acid N-cis-{4-[4-(2-isopropoxy- | 488 |
| | phenyl)-piperazin-1-yl]-cyclohexyl}-amide | |
| 61 | 5-Bromo-6-chloro-pyridine-3-sulfonic acid N-cis-{4-[4-(2-isopropoxy- | 571 |
| | phenyl)-piperazin-1-yl]-cyclohexyl}-amide | |
| 62 | N-trans-{4-[4-(2-Isopropoxy-phenyl)-piperazin-1-yl]-cyclohexyl}-C- | 471 |
| | phenyl-methanesulfonamide | |
| 63 | Quinoline-8-sulfonic acid N-trans-{4-[4-(2-isopropoxy-phenyl)- | 508 |
| | piperazin-1-yl]-cyclohexyl}-amide | |
| 64 | Pyridine-3-sulfonic acid N-trans-{4-[4-(2-isopropoxy-phenyl)- | 458 |
| | piperazin-1-yl]-cyclohexyl}-amide | |
| 65 | 4-Methoxy-pyridine-3-sulfonic acid N-trans-{4-[4-(2-isopropoxy- | 488 |
| | phenyl)-piperazin-1-yl]-cyclohexyl}-amide | |
| 66 | 5-Bromo-6-chloro-pyridine-3-sulfonic acid N-trans-{4-[4-(2- | 571 |
| | isopropoxy-phenyl)-piperazin-1-yl]-cyclohexyl}-amide | |

### EXAMPLE 2

### N-Ethyl-N-cis-{4-[4-(2-isopropoxy-phenyl)-piperazin-1-yl]-cyclohexyl}-3,4-dimethoxy-benzenesulfonamide (Cpd 67)

### N-Ethyl-N-trans-{4-[4-(2-isopropoxy-phenyl)-piperazin-1-yl]-cyclohexyl}-3,4-dimethoxy-benzenesulfonamide (Cpd 68)

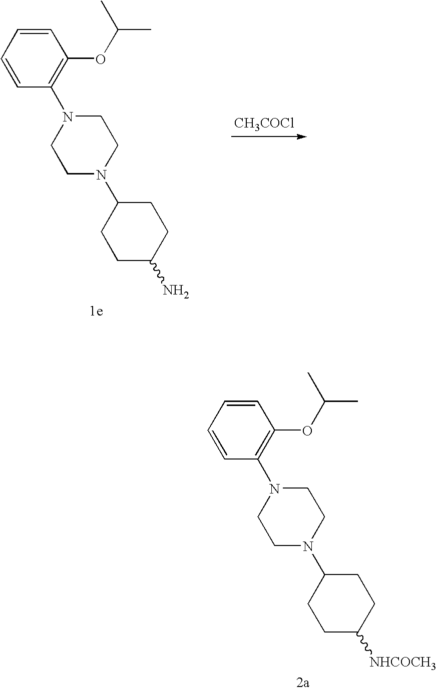

Acetyl chloride (37 mg, 0.472 mmol) was added to a solution of compound 1e (150 mg, 0.472 mmol) in CH₂Cl₂ (15 mL), then a 10% aqueous Na₂CO₃ solution (10 mL) was added and the mixture was stirred at rt for 1 hr. The organic layer was separated, dried and evaporated to give compound 2a (168 mg, 100%) as a yellowish oil (MS m/z 359 MH⁺).

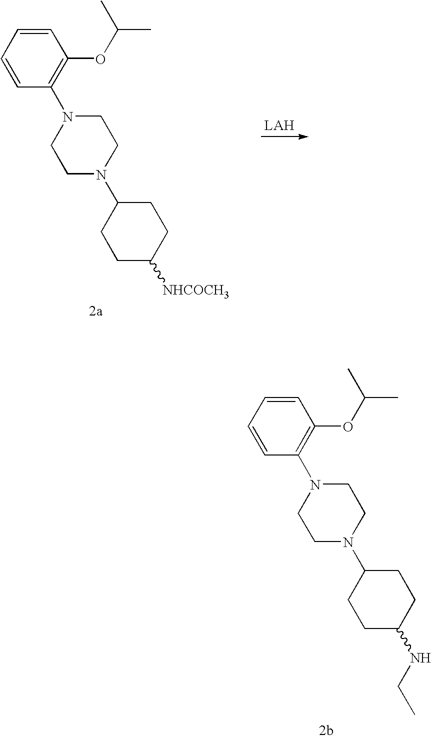

Compound 2a was treated with LiAlH₄ in THF and the mixture was refluxed for 10 hrs, cooled to rt and stirred with Na₂SO₄.10H₂O for 3 hrs. The mixture was filtered and evaporation of filtrate gave compound 2b (160 mg, 100%) as a yellowish oil which was used in the next step without further purification. (MS m/z 345 MH⁺).

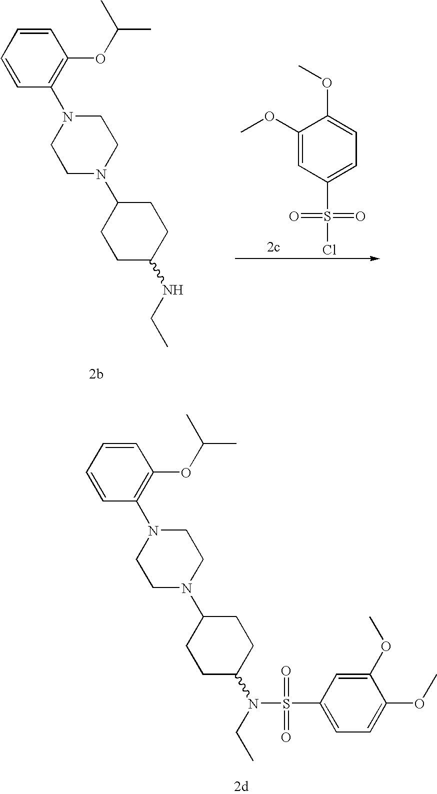

3,4-Dimethoxysulfonylchloride compound 2c (112 mg, 0.472 mmol) and a 10% aqueous solution of Na₂CO₃ (10 mL) were added to a solution of compound 2b in CH₂Cl₂ (25 mL). The mixture was stirred overnight at rt. The organic layer was separated and dried (Na₂SO₄) and the solvent was evaporated to provide compound 2d as a cis and trans isomer mixture. MS m/z 545 MH⁺.

Similar to Example 1, the isomers of compound 2d were separated and obtained through preparative-TLC-(7% 2M NH₃ in MeOH in CH₂Cl₂.) and converted to the difumarate salt.

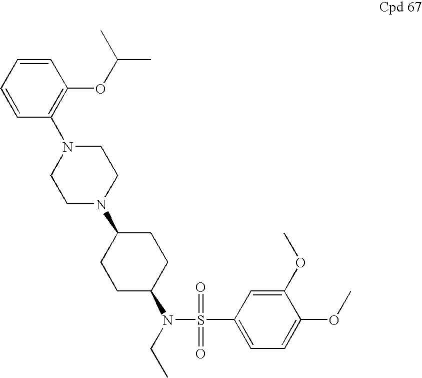

Compound 67 84 mg, 32.6%, m.p. 199° C. (dec), ¹H NMR (CDCl₃, TMS) δ 1.28 (t, J=7.8 Hz, 3H), 1.1-1.5 (m, 4H), 1.31 (d, J=6.5 Hz, 6H), 1.80 (m, 2H), 2.04 (bd, J=13 Hz, 2H), 2.15 (bs, 1H), 2.58 (bs, 4H), 3.06 (bs, 4H), 3.24 (q, J=7.8 Hz, 2H), 3.73 (m, 1H), 3.90 (s, 3H), 3.92 (s, 3H), 4.57 (m, 1H), 6.8-7.5 (m, 7H).

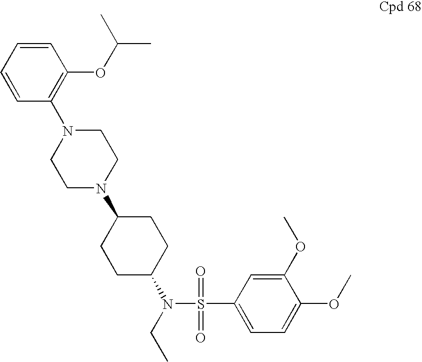

Compound 68 82 mg, 31.8%, m.p. 186° C. (dec), ¹H NMR (CDCl₃, TMS) δ 1.22 (t, J=7.8 Hz, 3H), 1.2-1.5 (m, 4H), 1.40 (d, J=6.5 Hz, 6H), 1.72 (bd, J=11.7 Hz 2H), 2.0 (bd, J=10.4 Hz, 2H), 2.25 (m, 1H), 2.71 (m, 4H), 3.08 (bs, 4H), 3.24 (q, J=7.8 Hz, 2H), 3.64 (m, 1H), 3.94 (s, 3H), 3.98 (s, 3H), 4.56 (m, 1H), 6.8-7.5 (m, 7H).

### EXAMPLE 3

### 3,4-Dimethoxy-N-cis-(4-{4-[2-(2,2,2-trifluoro-ethoxy)-phenyl]-piperazin-1-yl}-cyclohexyl)-benzenesulfonamide (Cpd 69)

### 3,4-Dimethoxy-N-trans-{4-[2-(2,2,2-trifluoro-ethoxy)-phenyl]-piperazin-1-yl}-cyclohexyl)-benzenesulfonamide (Cpd 70)

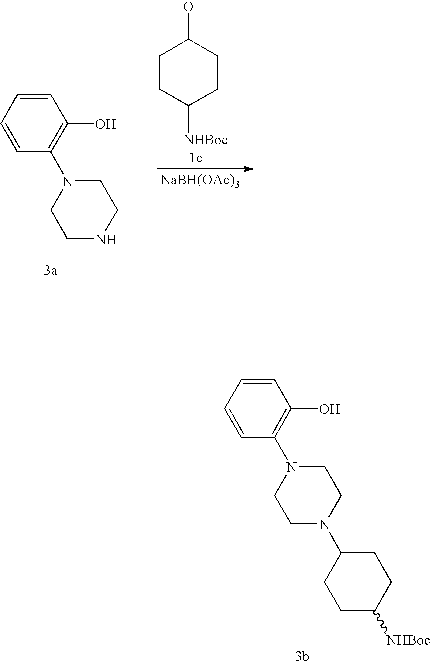

An aqueous solution of a 2-hydroxyphenylpiperazine compound 3a dihydrobromide salt (3.40 g, 10 mmol) was neutralized by one equivalent of K₂CO₃ and extracted by CH₂Cl₂. To the dried extracts was added N-Boc-4-aminocyclohexanone compound 1c (2.13 g, 10 mmol), NaBH(OAc)₃ (6.33 g, 30 mmol) and HOAc (0.5 mL). The mixture was stirred under N₂ for two days, then diluted with CH₂Cl₂, washed with water and dried (Na₂SO₄). The crude product was purified by short column chromatography to provide compound 3b (2.72 g, 72.5% yield) as a yellowish oil. MS m/z 375 MH⁺.

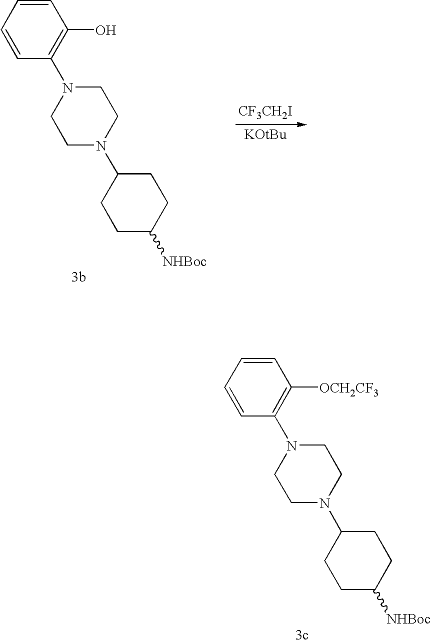

Potassium t-butoxy (KOtBu) (160 mg, 1.43 mmol) was added to a solution of compound 3b (536 mg, 1.43 mmol) in DMF (30 mL, dry). The mixture was stirred at rt for 40 minutes, then a solution of 3,3,3-trifluoro-1-iodoethane (299 mg, 1.43 mmol) in DMF (10 mL) was added dropwise. The mixture was stirred overnight at rt, then diluted with AcOEt (200 mL) and washed ten times with water. Evaporation of the solvent yielded a crude product which was purified by chromatography to provide compound 3c (190 mg, 29% yield) as a yellowish oil. MS m/z 457 MH⁺.

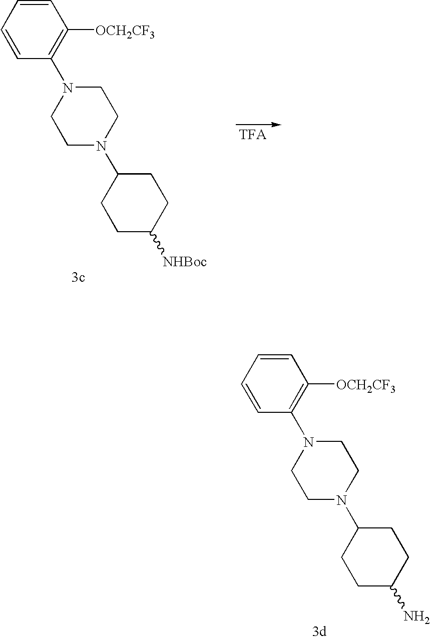

TFA (2 mL) was added to a solution of compound 3c (190 mg, 0.415 mmol) in CH₂Cl₂ (10 mL) at 0° C. and the mixture was stirred at rt for 2 hrs. All volatile materials were removed by evaporation to provide compound 3d as a crude product which was used in the next step without further purification.

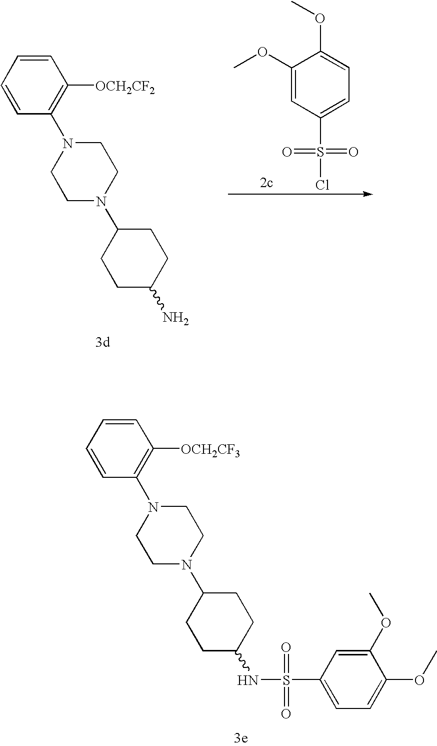

3,4-Dimethoxysulfonylchloride compound 2c (98 mg, 0.415 mmol) and a 10% aqueous solution of Na₂CO₃ (10 mL) was added to a solution of the crude compound 3d in CH₂Cl₂ (25 mL) and the mixture was stirred overnight at rt. The organic layer was separated and dried (Na₂SO₄) and solvent evaporation gave compound 3e as a crude product.

Similar to Example 1, the isomers of compound 3e were separated and obtained through repeated chromatography and converted to the difumarate salt.

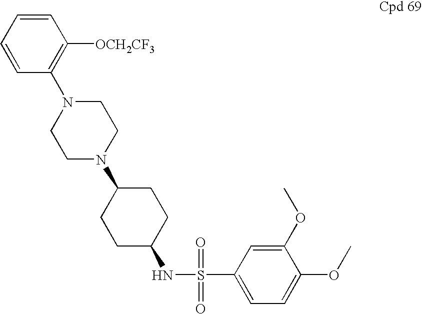

Compound 69 22 mg, 9.5%.

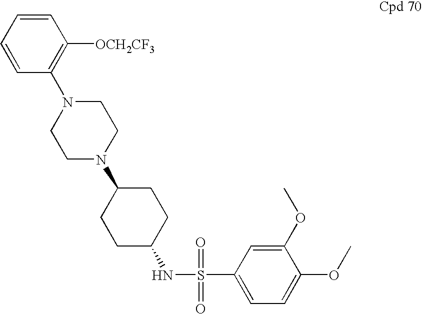

Compound 70 52 mg, 22%.

### EXAMPLE 4

### N-cis-{4-[4-(4-Fluoro-2-isopropoxy-phenyl)-piperazin-1-yl]-cyclohexyl}-3,4-dimethoxy-benzenesulfonamide (Cpd 71)

### N-trans-{4-[4-(4-Fluoro-2-isopropoxy-phenyl)-piperazin-1-yl]-cyclohexyl}-3,4-dimethoxy-benzenesulfonamide (Cpd 72)

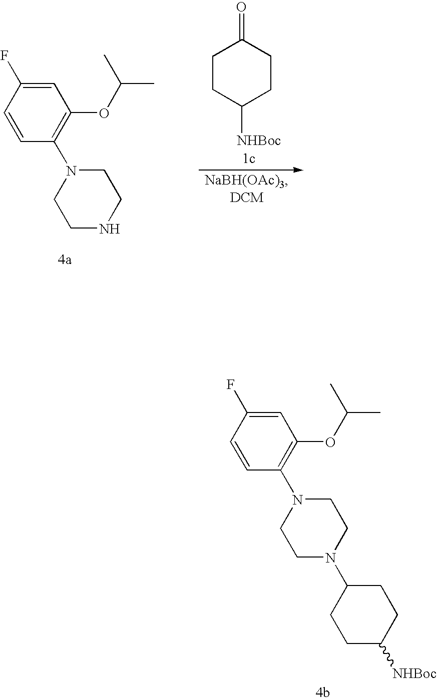

Compound 4a (0.24 g, 1 mmol), N-Boc-4-amino-cyclohexanone compound 1c (0.22 g, 1.05 mmol), NaBH(OAc)₃ (0.63 g, 3 mmol), HOAc (2 drops) and anhydrous DCM (25 mL) were mixed together, stirred under nitrogen atmosphere (white slurry became yellowish solution) at rt for 36 hrs, whereupon the reaction mixture was diluted with AcOEt (80 mL), washed with NaHCO₃ (sat.) and dried over Na₂SO₄. A crude product (reddish semi-solid) was obtained by solvent removal from the filtered dry solution using a rotary evaporator. Flash chromatography (100% AcOEt, silica gel) was used to provide a pure compound 4b (0.383 g, yield 88%) as a slightly red semi-solid. LC-MS at 3.006 minutes, m/z 436.2 MH⁺.

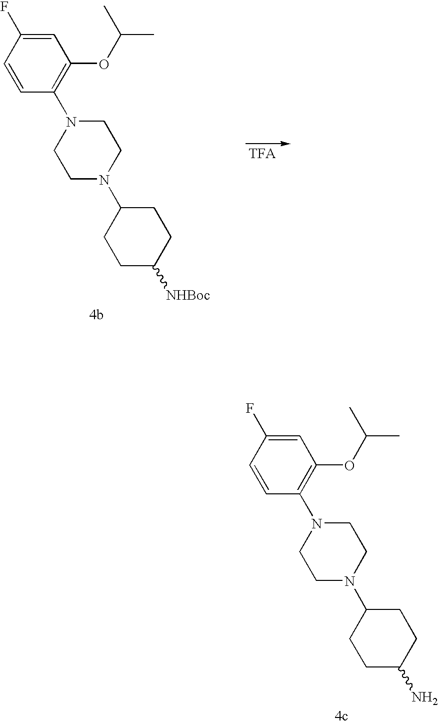

Compound 4b was dissolved into DCM and stirred with TFA (0.5 mL) at rt. The reaction was monitored by TLC (100% AcOEt), then the volatiles were removed on a rotary evaporator once the compound 4b starting material was consumed. The residue was mixed with DCM, treated with 1N NaOH, then the organic layer was dried over Na₂SO₄. Solvent evaporation from the dried solution provided compound 4c (0.227 g, 77.7%) as a yellowish oil which was used in the next step without further purification. LC-MS at 2.348 minutes, m/z 336.1 MH⁺

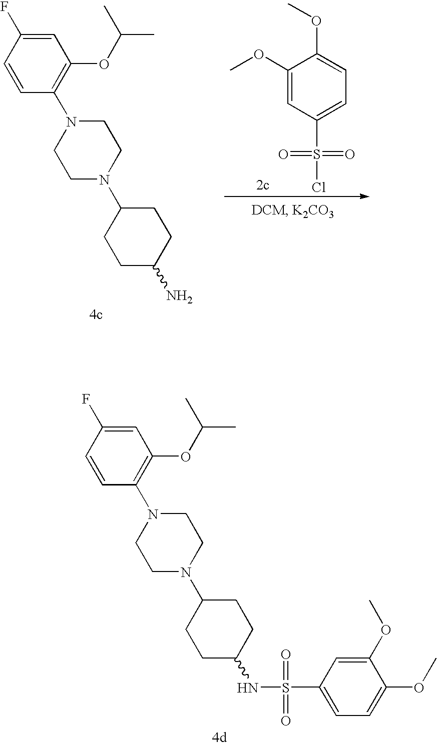

Compound 4c (0.030 g, 0.089 mmol) and 3,4-dimethoxybenzenesulfonyl chloride compound 2c (0.032 g, 0.135 mmol) were dissolved in DCM (2 mL), then K₂CO₃ (0.019 g) was added. The resulting yellowish turbid solution was stirred at rt and monitored by TLC (5% MeOH/DCM) and LC-MS. When compound 4c was no longer detected, the product solution containing compound 4d was filtered and the solution was loaded on preparative TLC plate which was developed using a solvent mixture (5% MeOH/DCM) to separate the isomeric mixture.

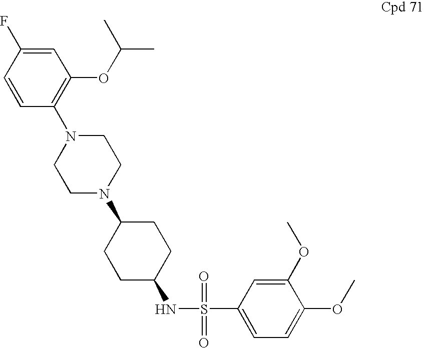

Compound 71 (0.013 g) was isolated as a yellowish oil from the less polar spot and was assigned as the cis isomer. LC-MS at 2.943 minutes, m/z 536.1 (100, M⁺). ¹H NMR (CDCl₃, TMS) δ 1.37 (d, J=6.4 Hz, 6H), 1.41-1.85 (m, 8H), 2.18-2.30 (m, 1H), 2.67-2.75 (m, 4H), 2.90-3.15 (m, 4H), 3.38-3.48 (m, 1H), 3.94 (s) & 3.96 (s, 6H), 4.50-4.63 (m, 1H), 4.84 (d, J=7.2 Hz, 1H), 6.50-6.65 (m, 2H), 6.75-6.86 (m, 1H), 6.94 (d, J=8.4 Hz, 1H), 7.39 (d, J=2.0 Hz, 1H), 7.52 (dd, J=2.0 Hz, J₂=8.4 Hz, 1H).

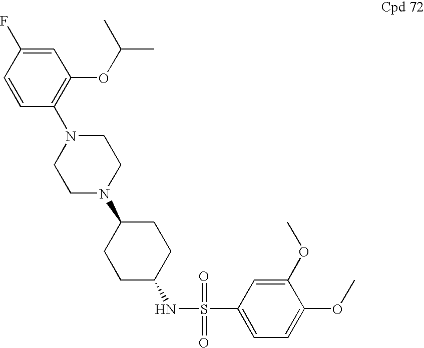

Compound 72 was isolated as a yellowish oil, (0.011 g) from the polar spot and was assigned as the trans isomer. LC-MS at 2.693 minutes, m/z 536.2 (100, M⁺). ¹H NMR (CDCl₃, TMS), δ 1.00-1.33 (m, 3H), 1.36 (d, J=6.0 Hz, 6H), 1.58-1.80 (m, 3H), 1.80-2.10 (m, 3H), 2.15-2.30 (m, 1H), 2.50-2.80 (m, 4H), 2.80-3.10 (m, 4H), 3.95 (s) & 3.97 (s, 6H), 4.46 (d, J=7.2 Hz, 1H), 4.50-4.65 (m, 1H), 6.45-6.70 (m, 2H), 6.70-6.86 (m, 1H), 6.95 (d, J=8.4 Hz, 1H), 7.36 (d, J=2.0 Hz, 1H), 7.52 (dd, J=2.0 Hz, J₂=8.4 Hz, 1H).

Using the procedure of Example 4, other compounds that are representative of the invention may be prepared by varying the starting materials, reagent(s) and conditions used (MS represents m/z of M⁺ or MH⁺):

| Cpd | Name | MS |
|---|---|---|
| 73 | 5-Chloro-N-cis-{4-[4-(4-fluoro-2-isopropoxy-phenyl)-piperazin-1-yl]- | 540 |
| | cyclohexyl}-2-methoxy-benzenesulfonamide | |
| 74 | 5-Chloro-2-fluoro-N-cis-{4-[4-(4-fluoro-2-isopropoxy-phenyl)- | 528 |
| | piperazin-1-yl]-cyclohexyl}-benzenesulfonamide | |
| 75 | N-cis-(4-{4-[4-Fluoro-2-(2,2,2-trifluoro-ethoxy)-phenyl]-piperazin-1- | 576 |
| | yl}-cyclohexyl)-3,4-dimethoxy-benzenesulfonamide | |
| 76 | 5-Chloro-N-cis-(4-{4-[4-fluoro-2-(2,2,2-trifluoro-ethoxy)-phenyl]- | 580 |
| | piperazin-1-yl}-cyclohexyl)-2-methoxy-benzenesulfonamide | |
| 77 | 5-Chloro-2-fluoro-N-cis-(4-{4-[4-fluoro-2-(2,2,2-trifluoro-ethoxy)- | 568 |
| | phenyl]-piperazin-1-yl}-cyclohexyl)-benzenesulfonamide | |
| 78 | 5-Chloro-N-trans-{4-[4-(4-fluoro-2-isopropoxy-phenyl)-piperazin-1- | 540 |
| | yl]-cyclohexyl}-2-methoxy-benzenesulfonamide | |
| 79 | 5-Chloro-2-fluoro-N-trans-{4-[4-(4-fluoro-2-isopropoxy-phenyl)- | 528 |
| | piperazin-1-yl]-cyclohexyl}-benzenesulfonamide | |
| 80 | N-trans-(4-{4-[4-Fluoro-2-(2,2,2-trifluoro-ethoxy)-phenyl]-piperazin-1- | 576 |
| | yl}-cyclohexyl)-3,4-dimethoxy-benzenesulfonamide | |
| 81 | 5-Chloro-N-trans-(4-{4-[4-fluoro-2-(2,2,2-trifluoro-ethoxy)-phenyl]- | 580 |
| | piperazin-1-yl}-cyclohexyl)-2-methoxy-benzenesulfonamide | |
| 82 | 5-Chloro-2-fluoro-N-trans-(4-{4-[4-fluoro-2-(2,2,2-trifluoro-ethoxy)- | 568 |
| | phenyl]-piperazin-1-yl}-cyclohexyl)-benzenesulfonamide | |

### EXAMPLE 5

### N-cis-(4-{4-[5-Fluoro-2-(2,2,2-trifluoro-ethoxy)-phenyl]-piperazin-1-yl}-cyclohexyl)-3,4-dimethoxy-benzenesulfonamide (Cpd 83)

### N-trans-(4-{4-[5-Fluoro-2-(2,2,2-trifluoro-ethoxy)-phenyl]-piperazin-1-yl}-cyclohexyl)-3,4-dimethoxy-benzenesulfonamide (Cpd 84)

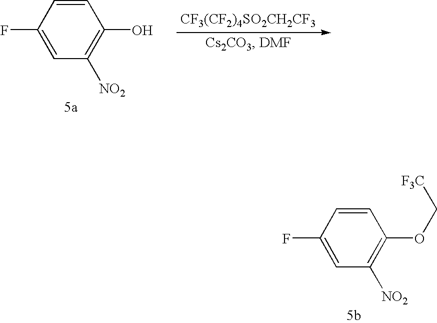

A solution of 2-nitro-4-fluorophenol compound 5a (1.00 g, 6.36 mmol) and Cs₂CO₃ (2.59 g, 7.95 mmol) in DMF (40 mL) was heated to 50° C. for 1 hr. The mixture was cooled to 35° C. and 2,2,2-trifluoroethyl 1,1,1,2,2,3,3,4,4-nonafluorobutanesulfonate (2.67 g, 7.00 mmol) was added. The mixture was stirred at rt overnight, diluted with AcOEt (200 mL), then washed ten times using water and dried. After evaporation, compound 5b (1.52 g, 100%) was obtained as a yellow oil.

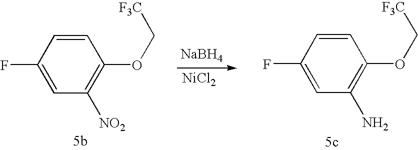

A solution of compound 5b (1.52 g, 6.36 mmol) and nickel chloride hexahydrate in a 6:1 mixture of MeOH:THF (60 mL:10 mL) was cooled in an ice bath and NaBH₄ (1.44 g, 38.16 mmol) was added portion wise. The mixture was stirred for 10 minutes at 0° C., then 1N HCl (4 mL) was added to quench the reaction. An aqueous ammonia solution (100 mL) and water (60 mL) were added and the mixture was stirred for 5 min at rt, then CH₂Cl₂ (100 mL) was added and the mixture was stirred vigorously for 30 min. The organic layer was separated and the aqueous layer extracted twice using CH₂Cl₂. The organic phase was combined and dried. Evaporation gave pure compound 5c (1.19 g, 89.4%) as a dark brown oil.

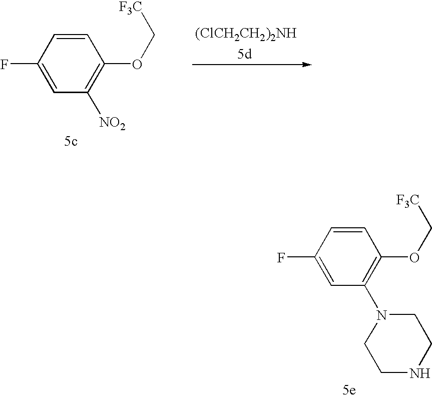

A suspension of compound 5c (1.10 g, 5.26 mmol), bis(2-chloroethyl)amine hydrochloride (also referred to as 2,2′-dichlorodiethylamine hydrochloride) compound 5d (948 mg, 5.31 mmol), Na₂CO₃ (557 mg, 5.26 mmol) and KI (1.05 g, 6.31 mmol) in n-BuOH (40 mL) was refluxed for 2 days. The reaction mixture was filtered and the solvent was evaporated from the filtrate, then the residue was dissolved in CH₂Cl₂, washed using water and dried to provide a crude product. The crude product was purified via repeated chromatography to provide compound 5e (100 mg, 6.8%) as a colorless oil.

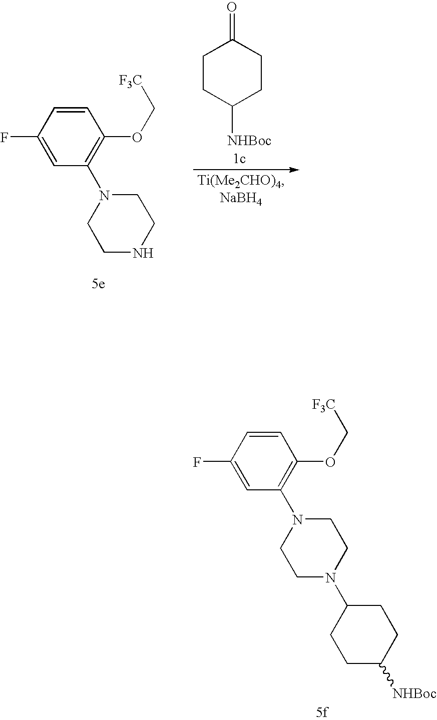

Ti(Me₂CHO)₄ (446 mg, 1.57 mmol) was added to a solution of compound 5e (291 mg, 1.046 mmol) and N-Boc-4-aminocyclohexanone compound 1c (223 mg, 1.046 mmol) in CH₂Cl₂ (15 ml). The mixture was stirred overnight at rt under nitrogen, then NaBH₄ (300 mg, 7.93 mmol) was added and the mixture was stirred for 4 hrs at rt. The mixture was carefully quenched by MeOH. All solvents were evaporated, then the solid residue was treated with CH₂Cl₂ (200 ml) and filtered. The filtrate was washed using a 10% solution of Na₂CO₃ and dried to provide a crude product. The crude product was purified via chromatography to provide compound 5f (279 mg, 56.1%) which was used directly in the next step.

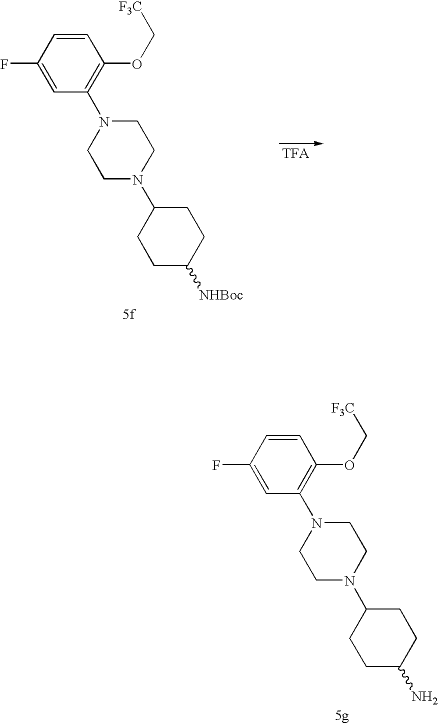

A solution of compound 5f (129 mg, 0.271 mmol) in CH₂Cl₂ (10 mL) was treated with TFA (5 mL) at 0° C. The mixture was stirred for 2 hrs at rt. All solvents were evaporated and a crude product compound 5g was used in the next step without further purification.

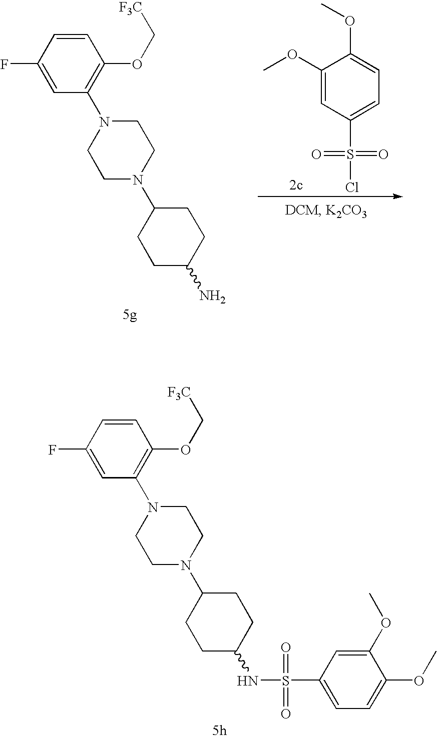

3,4-Dimethoxysulfonylchloride compound 2c (64 mg, 0.271 mmol) was added to a solution of compound 5g in CH₂Cl₂ (10 mL). The mixture was stirred for 5 min at rt, then a 10% aqueous solution of Na₂CO₃ (10 mL) was added. The mixture was stirred overnight at rt. The organic layer was separated and dried (Na₂SO₄), then solvents were evaporated to provide a crude product compound 5h as a mixture of isomers. MS m/z 575 MH⁺. The product compound 5h isomeric mixture was separated using a SiO₂ column (MeOH/CH₂Cl₂ 1-3% elusion) to provide the cis isomer compound 83 (73 mg, 46.8%) as a white powder and the trans isomer compound 84.

Fumaric acid (14 mg, 1 eq) was added to a mixture of compound 83 in MeOH/Et₂O to precipitate the fumarate salt of compound 83. The fumarate salt of compound 84 was similarly prepared.

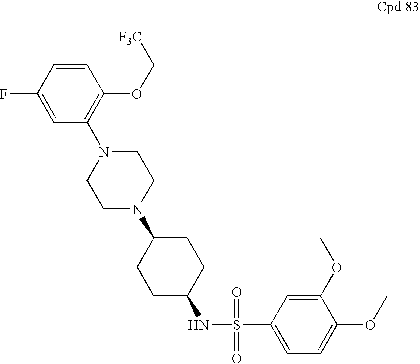

Compound 83 m.p. 232° C.; ¹H NMR (CDCl₃, TMS) δ 1.40-1.90 (m, 8H), 2.25 (m, 1H), 2.64 (bs, 4H), 3.06 (bs, 4H), 3.40 (bs, 1H), 3.92 (s, 3H), 3.95 (s, 3H), 4.35 (q, J=9.1 Hz, 2H), 5.04 (m, 1H), 6.5-7.6 (m, 6H).

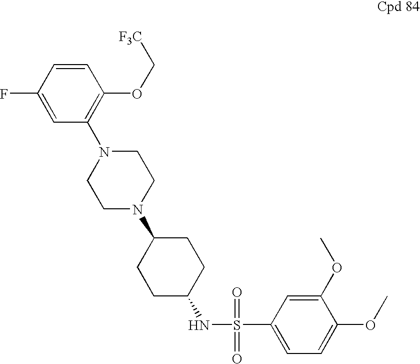

Compound 84 m.p. 185° C.; ¹H NMR (CDCl₃, TMS) δ1.1˜1.4 (m, 4H), 1.94 (m, 4H), 2.24 (m, 1H), 2.65 (m, 4H), 3.05 (bs, 5H), 3.92 (s, 3H), 3.95 (s, 3H), 4.34 (q, J=10.4 Hz, 2H), 4.54 (d, J=7 Hz, 1H), 6.6˜7.6, (m, 6H).

Additional compounds may be made according to the synthetic methods of the present invention by one skilled in the art, differing only in possible starting materials, reagents and conditions used in the instant methods.

### BIOLOGICAL EXAMPLES

The ability of the compounds to treat or ameliorate protein kinase mediated disorders was determined using the following procedures.

### Biological Example 1

α₁-Adrenergic Receptor Binding Assay: Preparation of COS Cell Membranes

Membranes were prepared from COS-7 cells that had been transfected with one of the three α₁-AR subtypes by the following method. COS cells from ten 100 mm tissue culture plates were scraped into 5 mL TE (50 mM Tris-HCl, 5 mM EDTA, pH 7.4). The cell suspension was disrupted with a Brinkman Polytron, setting 8, for 10 sec. The disrupted cells were centrifuged at 1000×g for 10 min at 4° C. Supernatants were centrifuged at 34,500×g for 20 min at 4° C. The membrane pellets were suspended in 2 mL TNE (50 mM Tris-HCl, 5 mM EDTA, 150 mM NaCl, pH7.4). An aliquot of the membrane suspension was stored at −70° C. until use. The protein concentration was determined using the BioRad DC protein assay kit following membrane solubilization with Triton X-100.

Radio-Ligand Binding Assay

Triplicate determinations of radio-ligand binding in the presence of increasing: concentrations of testing compound were made. The reagents were added to 96-well polypropylene plate wells using the Biomek 1000 robot (Beckman Instruments). Each assay well contained 140 μl TNE, 25 μL ¹²⁵I-HEAT (specific activity 2200 Ci/mmol, Dupont-NEN, 50 pM final), 10 μL testing compound dissolved in DMSO (1 pM to 10 μM in half-log increments, final), and 25 μL appropriate α₁-AR membrane subtype suspension in TNE (0.5 ng/μL for the α₁ₐ and α_{1b} subtypes and 13 ng/μL for the α_{1d} subtype). The plate was incubated at rt for 1 hr. The contents of the wells were filtered through a GF/C membrane Unifilter plate (Packard Instruments) using the Packard Filtermate cell harvester. The filter plates were dried in a vacuum oven for 30 min at 40° C. 25 μL Microscint 20 liquid scintillation fluid (Packard Instuments) was added to each well. The radioactive content was analyzed in the TopCount microplate scintillation counter (Packard Instruments).

Data Analysis

The Kᵢ values (in nM) shown in Table 1 were determined using GraphPad Prism software. K_{d} values used in the Kᵢ calculation for the α₁-AR subtypes for ¹²⁵I-HEAT were 81.5 nM for the α₁ₐ-AR, 79 nM for the α₁b-AR and 50 nM for the α_{1d}-AR.

TABLE 1
Receptor Binding, Kᵢ (nM)

| Cpd | α₁ₐ-AR | α₁b-AR | α_{1d}-AR |
|---|---|---|---|
| 1 | 4.5 | 177 | 0.64 |
| 2 | 12.4 | 276 | 4.2 |
| 3 | 2.5 | 47.6 | 0.22 |
| 4 | 4.6 | 141 | 3.8 |
| 5 | 12.5 | 118 | 2.8 |
| 6 | 4.8 | 195 | 3.4 |
| 7 | 1.6 | 109 | 1.0 |
| 8 | 11.8 | 167 | 9.6 |
| 9 | 25 | 124 | 18 |
| 10 | 5.6 | 150 | 4.8 |
| 11 | 18 | 129 | 1.1 |
| 12 | 3.8 | 66 | 0.4 |
| 13 | 23 | 190 | 3.4 |
| 14 | 11.6 | 246 | 4.3 |
| 15 | 6 | 162 | 5 |
| 16 | 14 | 222 | 1.4 |
| 17 | 17.4 | 174 | 2.1 |
| 18 | 10.5 | 167 | 11.1 |
| 19 | 4.3 | 85.3 | 2.2 |
| 20 | 35.9 | 473 | 14 |
| 21 | 5.6 | 103 | 2.7 |
| 22 | 3.5 | 108 | 3.3 |
| 23 | 6.6 | 195 | 3 |
| 24 | 11 | 101 | 1.5 |
| 25 | 13.7 | 111 | 5.2 |
| 26 | 6.3 | 201 | 2.5 |
| 27 | 9.6 | 217 | 6.3 |
| 28 | 1.6 | 42 | 0.8 |
| 29 | 1.6 | 596 | 8.6 |
| 30 | 5.0 | 204 | 49 |
| 31 | 3.8 | 250 | 11 |
| 32 | 0.9 | 218 | 31 |
| 33 | 12.5 | 118 | 2.8 |
| 34 | 7.5 | 392 | 22 |
| 35 | 23 | 126 | 24 |
| 36 | 17 | 424 | 56 |
| 37 | 67 | 931 | 65 |
| 38 | 1.3 | 301 | 36 |
| 39 | 1.1 | 554 | 5.4 |
| 40 | 8.9 | 441 | 27.3 |
| 41 | 18.1 | 844 | 246 |
| 42 | 6.6 | 2301 | 72 |
| 43 | 11 | 714 | 66 |
| 44 | 19.7 | 334 | 34.7 |
| 45 | 100 | 1254 | 111 |
| 46 | 1.5 | 193 | 16 |
| 47 | 1 | 173 | 22 |
| 48 | 24.5 | 258 | 1.5 |
| 49 | 0.88 | 385 | 30 |
| 50 | 0.73 | 545 | 24.5 |
| 51 | 0.56 | 348 | 24 |
| 52 | 3 | 243 | 19 |
| 53 | 2.7 | 487 | 55 |
| 54 | 1 | 236 | 27 |
| 55 | 0.77 | 31 | 3.2 |
| 56 | 10 | 131 | 38 |
| 57 | 1.2 | 82 | 3.6 |
| 58 | 8.3 | 116 | 4.3 |
| 59 | 5 | 400 | 46 |
| 60 | 4.9 | 169 | 9.4 |
| 61 | 14.4 | 125 | 0.14 |
| 62 | 3.9 | 200 | 12 |
| 63 | 0.5 | 77.6 | 22.1 |
| 64 | 5.8 | 192 | 5.5 |
| 65 | 13.4 | 268 | 76 |
| 66 | 3.5 | 279 | 14.6 |
| 67 | 15 | 326 | 22 |
| 68 | 23 | 109 | 6.7 |
| 69 | 1.2 | 100 | 0.92 |
| 70 | 54.2 | 34.8 | 8.3 |
| 71 | 14.9 | 711 | 5.9 |
| 72 | 9.0 | 146 | 12.2 |
| 73 | 598 | 81 | 134 |
| 74 | 5.6 | 101 | 2.2 |
| 75 | 13.9 | 176 | 5.2 |
| 76 | 17 | 196 | 8.5 |
| 77 | 43 | 206 | 29 |
| 78 | 340 | 44 | 47 |
| 79 | 5.4 | 107 | 14 |
| 80 | 21 | 87 | 38 |
| 81 | 34 | 90 | 51 |
| 82 | 52 | 442 | 240 |
| 83 | 1.7 | 75 | 0.58 |
| 84 | 33 | 220 | 36.1 |

### Biological Example 2

In-Vivo Evaluation of α₁-Adrenergic Antagonists on Prostatic Intraurethral Pressure (IUP) and Mean Arterial Pressure (MAP) in an Anesthetized Canine Model

An anesthetized dog was fitted with a balloon catheter for the measurement of changes in prostatic IUP and with a non-invasive blood pressure (NIBP) cuff for determination of MAP.

The pressor effects of challenge doses (i.v.) of the α-adrenergic agonist phenylephrine upon IUP and MAP were measured before and at various time points after a single dose (i.v.) of an α-adrenergic antagonist test compound (Compound 69) of the present invention.

Each treatment group was dosed with a test compound (0.01, 0.03, 0.1 and 0.5 mg/kg) in four separate experiments. The first challenge dose of phenylephrine was administered 30 mins prior to the test compound dose and at 30 min intervals thereafter for a total time period of 5 hrs (i.e at time minus 30 mins, time plus 30 mins, 1 hr, 1.5 hrs, 2 hrs, 2.5 hrs, 3 hrs, 3.5 hrs, 4 hrs, 4.5 hrs and 5 hrs).

The animals were fasted overnight, anesthetized with propofol (4 mg/kg IV or to effect), then intubated and anesthesia maintained using inhalation isoflurane (1-2% in oxygen at a flow rate of 2 l/min) with animals breathing spontaneously.

A 7F Fogarty balloon catheter lubricated with a water-soluble jelly was inserted into the urethra and advanced into the bladder (approximately 40 cm). The balloon was inflated with approximately water (0.2 mL). The catheter was slowly withdrawn from the bladder to just past the point of first resistance from the bladder neck in order to position the balloon within the prostatic urethra. The balloon was then inflated to approximately 0.7 mL. The balloon port of the catheter was connected to a Gould Statham pressure transducer, the output of which was connected to the pressure input of a Physio Control VSM1 Patient Monitor. The pressure analog output signal from the VSM1 was displayed on a Kipp & Zonen Type BD112 flatbed recorder.

Blood pressure was measured non-invasively using a Critikon 8100 monitor. An infant blood pressure cuff was placed on a forelimb. Measurements were made automatically at 1-minute intervals during recording periods. Digital values for MAP displayed on the monitor were recorded by hand. Care was taken to accurately correlate the time of the MAP readings with the time of the phenylephrine dose. Recording periods typically lasted from 5 minutes before phenylephrine administration to 10 minutes after. The monitor was turned off between recording periods to allow the arterial blood vessels of the forelimb to recover from repeated cuff inflations.

Measurement of MAP:

Because of the variability of the MAP value, several values recorded during a five minute “control” period prior to the phenylephrine challenge dose were averaged to establish a mean baseline value for measuring the phenylephrine-induced change in MAP.

MAP readings also varied during the response to the phenylephrine challenge. A visual peak MAP value, seen on the monitor at about 3 minutes after the challenge dose, was recorded and used to calculate the maximum phenylephrine induced change in MAP. Additionally, MAP values were recorded at one minute intervals and curve fitted to estimate a maximum MAP value. The curve fitted maximum value was used to confirm the visual maximum value.

Administration of Challenge and Test Compound Doses:

A test compound was administered via the cephalic vein using an appropriately sized IV catheter (Surflo, Terumo Medical Corporation or equivalent).

Prior to test compound administration, IUP and MAP responses to an individualized dose of phenylephrine (either 10 or 15 μg/kg IV, depending on the individual animal's response) was repeated 2 to 3 times to establish a baseline response. After test compound administration, the individualized phenylephrine dose was administered to each animal at 30 minute intervals over a period of 5 hours.

Data Analysis

Data for phenylephrine-induced changes in IUP and MAP were tabulated for each treatment group and each experiment. A mean value and standard deviation for the phenylephrine-induced changes to the effect of a test compound on IUP and MAP at each dose was calculated and tabulated for each treatment group over the length of the 5.5 hour study.

Results

The mean values for % inhibition of IUP by Compound 69 at each test dose are shown in Table 1 and demonstrate that a compound of the present invention is dose dependently useful for reducing IUP. NA represents that a challenge dose was not administered.

TABLE 1
Test Compound IUP, normalized % of baseline (mean)

| | Time after Test Compound Dose (hrs) | | | | | | | | | | |
|---|---|---|---|---|---|---|---|---|---|---|---|
| Dose | 0 | 0.5 | 1 | 1.5 | 2 | 2.5 | 3 | 3.5 | 4 | 4.5 | 5 |
| 0.01 | 100 | 78.1 | 81.9 | NA | 83.6 | NA | 83.3 | NA | 83.0 | NA | 84.5 |
| 0.03 | 100 | 64.8 | 64.8 | 70.8 | 71.1 | 79.1 | 81.1 | 81.6 | 84.8 | 83.5 | 83.8 |
| 0.1 | 100 | 39.5 | 48.4 | 56.3 | 61.3 | 67.1 | 69.9 | 75.2 | 78.8 | 74.2 | 77.3 |
| 0.5 | 100 | 17.4 | 16.1 | 27.0 | 29.1 | 36.1 | 38.8 | 45.5 | 46.4 | 48.0 | 54.4 |

The mean values for % inhibition of MAP by Compound 69 at each test dose are shown in Table 2 and demonstrate that a compound of the present invention is dose dependently useful for reducing MAP. NA represents that a challenge dose was not administered.

TABLE 2
Test Compound MAP, normalized % of baseline (mean)

| | Time after Test Compound Dose (hrs) | | | | | | | | | | |
|---|---|---|---|---|---|---|---|---|---|---|---|
| Dose | 0 | 0.5 | 1 | 1.5 | 2 | 2.5 | 3 | 3.5 | 4 | 4.5 | 5 |
| 0.01 | 100 | 88.2 | 68.9 | NA | 83.1 | NA | 88.0 | NA | 82.0 | NA | 91.1 |
| 0.03 | 100 | 39.9 | 40.9 | 44.1 | 61.0 | 59.4 | 59.2 | 59.8 | 58.4 | 71.1 | 90.3 |
| 0.1 | 100 | 45.5 | 47.4 | 35.0 | 44.0 | 54.0 | 51.3 | 56.4 | 47.0 | 73.3 | 84.3 |
| 0.5 | 100 | 27.6 | 18.4 | 16.0 | 18.3 | 19.4 | 23.8 | 19.2 | 32.3 | 32.1 | 29.4 |

### Biological Example 3

In-Vivo Evaluation of α₁-Adrenergic Antagonists on IUP and MAP in a Conscious Canine Model

An anesthetized dog was fitted with a balloon catheter for the measurement of changes in prostatic IUP and with a number of implanted pressure transducers and telemetry transmitters for measuring MAP.

The pressor effects of challenge doses (p.o.) of the α-adrenergic agonist phenylephrine upon IUP and MAP were measured before and at various time points after a single dose (p.o.) of an α-adrenergic antagonist test compound (Compound 69) of the present invention.

Each treatment group was dosed with a test compound (0.1, 0.3, 1.0 and 3.0 mg/kg) in four separate experiments. The first challenge dose of phenylephrine for each treatment group was administered at the time of the test compound dose (time 0). The value for the first challenge dose represents an average of up to three challenge doses administered prior to time 0.

Subsequent challenge doses for all treatment groups were administered at 30 min. then one hour and two hours after time 0.

The final challenge doses for the 0.1 and 0.3 mg/kg groups were administered at four and six hours after time 0. The final challenge doses for the 1.0 mg/kg group were administered at four, six and eight hours after time 0. The final challenge doses for the 3.0 mg/kg group were administered at four, six, 12 and 24 hours after time 0.

Preoperative Preparation (Telemetry Implant):

The animals were fasted overnight, then sedated and anesthesized. Vital signs were monitored during anesthesia.

MAP Measurement Instrumentation:

A pressure transducer and telemetry transmitter (DSI TA11PA-D70 35 cm, Data Sciences International, St. Paul, Minn.) were implanted according to directions in “PA Device Surgical Manual” provided by the manufacturer.

IUP Measurement Instrumentation:

A 7F Fogarty balloon catheter lubricated with a water-soluble jelly was inserted into the urethra and advanced into the bladder (approximately 40 cm). The balloon was inflated with approximately water (0.2 mL). The catheter was slowly withdrawn from the bladder to just past the point of first resistance from the bladder neck in order to position the balloon within the prostatic urethra. The balloon was then inflated to approximately 0.7 mL. The balloon port of the catheter was connected to a Gould Statham pressure transducer, the output of which was connected to the pressure input of a Physio Control VSM1 Patient Monitor. The pressure analog output signal from the VSM1 was displayed on a Kipp & Zonen Type BD112 flatbed recorder.

Administration of Challenge and Test Compound Doses:

A test compound was administered via the cephalic or saphenous vein using an appropriately sized IV catheter (Surflo, Terumo Medical Corporation or equivalent).

Prior to test compound administration, IUP and MAP responses to an individualized dose of phenylephrine (either 10 or 15 μg/kg IV, depending on the individual animal's response) was repeated 2 to 3 times to establish a baseline response. After test compound administration, the individualized phenylephrine dose was administered to each animal at predetermined intervals over a period of up to 24 hours.

Data Analysis

Data for phenylephrine-induced changes in IUP and MAP were tabulated for each treatment group and each experiment. A mean value and standard deviation for the phenylephrine-induced changes to the effect of a test compound on IUP and MAP at each dose was calculated and tabulated for each treatment group over the length of the study.

Results

The mean values for % inhibition of IUP by Compound 69 at each test dose are shown in Table 3 and demonstrate that a compound of the present invention is dose dependently useful for reducing IUP. NA represents that a challenge dose was not administered.

TABLE 3
Test Compound IUP, normalized % of baseline (mean)

| | Time after Test Compound Dose (hrs) | | | | | | | | |
|---|---|---|---|---|---|---|---|---|---|
| Dose | 0 | 0.5 | 1 | 2 | 4 | 6 | 8 | 12 | 24 |
| 0.1 | 100 | 87.3 | 78.3 | 78.8 | 82.5 | 86.5 | NA | NA | NA |
| 0.3 | 100 | 63.0 | 47.0 | 51.1 | 70.0 | 84.4 | NA | NA | NA |
| 1.0 | 100 | 32.9 | 20.6 | 28.6 | 49.0 | 66.3 | 70.4 | NA | NA |
| 3.0 | 100 | 5.2 | 2.2 | 7.0 | 17.1 | 31.6 | NA | 67.7 | 93.8 |

The mean values for % inhibition of MAP by Compound 69 at each test dose are shown in Table 4 and demonstrate that a compound of the present invention is dose dependently useful for reducing MAP. NA represents that a challenge dose was not administered.

TABLE 4
Test Compound MAP, normalized % of baseline (mean)

| | Time after Test Compound Dose (hrs) | | | | | | | | |
|---|---|---|---|---|---|---|---|---|---|
| Dose | 0 | 0.5 | 1 | 2 | 4 | 6 | 8 | 12 | 24 |
| 0.1 | 100 | 87.7 | 93.3 | 80.1 | 89.7 | 91.0 | NA | NA | NA |
| 0.3 | 100 | 64.9 | 59.3 | 67.7 | 85.6 | 103.9 | NA | NA | NA |
| 1.0 | 100 | 47.3 | 37.2 | 52.8 | 61.3 | 76.6 | 87.8 | NA | NA |
| 3.0 | 100 | 43.4 | 19.7 | 21.7 | 39.8 | 50.2 | NA | 80.2 | 95.1 |

It is to be understood that the preceding description teaches the principles of the present invention, with examples thereof, which have emphasized certain aspects. It will also be understood that the practice of the invention encompasses all of the usual variations, adaptations and modifications as come within the scope of the following claims and their equivalents. However, numerous other equivalents not specifically elaborated on or discussed may nevertheless fall within the spirit and scope of the present invention and claims and are intended to be included.

Throughout this application, various publications are cited. The disclosure of all publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats.

What is claimed is:

1. A compound of formula (I) 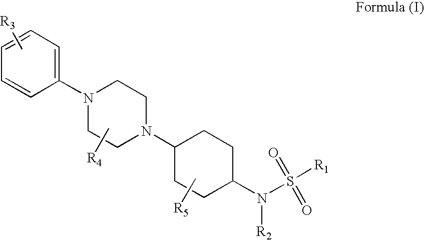
and pharmaceutically acceptable forms thereof, wherein
R₁ is selected from the group consisting of
(1) aryl,
(2) C₁₋₈alkyl(aryl),
(3) C₃₋₈cycloalkyl,
(4) C₁₋₈alkyl(C₃₋₈cycloalkyl),
(5) heteroaryl,
(6) C₁₋₈alkyl(heteroaryl),
(7) heterocyclyl, and
(8) C₁₋₈alkyl(heterocyclyl),
wherein (1), (3), (5) and (7) and the aryl, C₃₋₈cycloalkyl, heteroaryl and heterocyclyl portions of (2), (4), (6) and (8) respectively are optionally substituted with up to four substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂ substituted on sulfur with a substituent selected from the group consisting of C₁₋₈alkyl, C₃₋₈cycloalkyl, aryl, heteroaryl, or heterocyclyl,
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl), (xvi) C₁₋₈alkoxy(aryl),
(xvii) C₁₋₈alkyl(heteroaryl),
(xviii) C₁₋₈alkyl(heterocyclyl);
(xix) CO substituted on carbon with a substituent selected from the group consisting of hydrogen, C₁₋₈alkyl, C₃₋₈cycloalkyl, aryl, heteroaryl, or heterocyclyl,
(xx) SO substituted on sulfur with a substituent selected from the group consisting of C₁₋₈alkyl, C₃₋₈cycloalkyl, aryl, heteroaryl, or heterocyclyl,
(xxi) C(O)N substituted on nitrogen with two substituents selected from the group consisting of hydrogen, C₁₋₈alkyl, C₃₋₈cycloalkyl, aryl, heteroaryl, or heterocyclyl,
(xxii) SO₂N substituted on nitrogen with two substituents selected from the group consisting of hydrogen, C₁₋₈alkyl, C₃₋₈cycloalkyl, aryl, heteroaryl, or heterocyclyl,
(xxiii) NHSO₂ substituted on sulfur with a substituent selected from the group consisting of C₁₋₈alkyl, C₃₋₈cycloalkyl, aryl, heteroaryl, or heterocyclyl,
(xxiv) NH(CO) substituted on carbon with a substituent selected from the group consisting of hydrogen, C₁₋₈alkyl, C₃₋₈cycloalkyl, aryl, heteroaryl, or heterocyclyl,
(xxv) NHSO₂N substituted on nitrogen with two substituents selected from the group consisting of hydrogen, C₁₋₈alkyl, C₃₋₈cycloalkyl, aryl, heteroaryl, or heterocyclyl,
(xxvi) NH(CO)N substituted on nitrogen with two substituents selected from the group consisting of hydrogen, C₁₋₈alkyl, C₃₋₈cycloalkyl, aryl, heteroaryl, or heterocyclyl,
(xxvii) C₃₋₈cycloalkyl,
(xxviii) aryl,
(xxix) heteroaryl, and
(xxx) heterocyclyl;
R₂ is selected from the group consisting of hydrogen and C₁₋₈alkyl;
R₃ is up to four optionally present substituents independently selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂ substituted on sulfur with a substituent selected from the group consisting of C₁₋₈alkyl, C₃₋₈cycloalkyl, aryl, heteroaryl, or heterocyclyl,
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl,
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl),
(25) CO substituted on carbon with a substituent selected from the group consisting of hydrogen, C₁₋₈alkyl, C₃₋₈cycloalkyl, aryl, heteroaryl, or heterocyclyl,
(26) SO substituted on sulfur with a substituent selected from the group consisting of C₁₋₈alkyl, C₃₋₈cycloalkyl, aryl, heteroaryl, or heterocyclyl,
(27) SO₂ substituted on sulfur with a substituent selected from the group consisting of C₁₋₈alkyl, C₃₋₈cycloalkyl, aryl, heteroaryl, or heterocyclyl,
(28) C(O)N substituted on nitrogen with two substituents selected from the group consisting of hydrogen, C₁₋₈alkyl, C₃₋₈cycloalkyl, aryl, heteroaryl, or heterocyclyl,
(29) SO₂N substituted on nitrogen with two substituents selected from the group consisting of hydrogen, C₁₋₈alkyl, C₃₋₈cycloalkyl, aryl, heteroaryl, or heterocyclyl,
(30) NHSO₂ substituted on sulfur with a substituent selected from the group consisting of C₁₋₈alkyl, C₃₋₈cycloalkyl, aryl, heteroaryl, or heterocyclyl,
(31) NH(CO) substituted on carbon with a substituent selected from the group consisting of hydrogen, C₁₋₈alkyl, C₃₋₈cycloalkyl, aryl, heteroaryl, or heterocyclyl,
(32) NHSO₂N substituted on nitrogen with two substituents selected from the group consisting of hydrogen, C₁₋₈alkyl, C₃₋₈cycloalkyl, aryl, heteroaryl, or heterocyclyl, and
(33) NH(CO)N substituted on nitrogen with two substituents selected from the group consisting of hydrogen, C₁₋₈alkyl, C₃₋₈cycloalkyl, aryl, heteroaryl, or heterocyclyl
(34) C₃₋₈cycloalkoxy;
wherein (15), (18), (21) and (23) and the aryl, C₃₋₈cycloalkyl, heteroaryl and heterocyclyl portions of (8), (16), (17), (19), (20), (22), (24), (25), (26), (27), (28), (29), (30), (31), (32), (33), and (34) are optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro, and
(xiv) C₁₋₈alkyl(amino) optionally mono- or di-substituted on amino with C₁₋₈alkyl; and
R₄ and R₅ are up to two optionally present substituents independently selected from the group consisting of oxo and C₁₋₆alkyl.

2. A compound according to claim 1, wherein R₁ is selected from the group consisting of
(1) aryl,
(2) C₁₋₈alkyl(aryl), and
(3) heteroaryl,
wherein (1) and (3) and the aryl portion of (2) is optionally substituted with up to four substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono- or di-substituted on amino with C₁₋₈alkyl,
(xv) C₁₋₈alkyl(aryl),
(xvi) C₁₋₈alkoxy(aryl)
(xvii) C₁₋₈alkyl(heteroaryl), and
(xviii) C₁₋₈alkyl(heterocyclyl);
R₂ is selected from the group consisting of hydrogen and C₁₋₈alkyl; and
R₃ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl,
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl), and
(25) C₃₋₈cycloalkoxy.

3. A compound according to claim 1, wherein the compound is a compound of Formula (II) 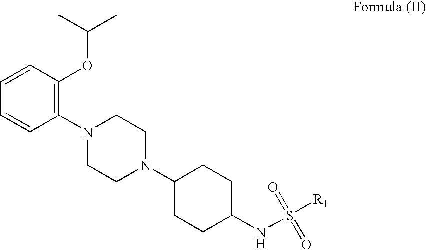
and pharmaceutically acceptable forms thereof, wherein
R₁ is selected from the group consisting of
(1) aryl,
(2) C₁₋₈alkyl(aryl),
(3) C₃₋₈cycloalkyl,
(4) C₁₋₈alkyl(C₃₋₈cycloalkyl),
(5) heteroaryl,
(6) C₁₋₈alkyl(heteroaryl),
(7) heterocyclyl, and
(8) C₁₋₈alkyl(heterocyclyl),
wherein (1), (3), (5) and (7) and the aryl, C₃₋₈cycloalkyl, heteroaryl and heterocyclyl portions of (2), (4), (6) and (8) respectively are optionally substituted with from one to two substituents independently selected from the group consisting of
(i) C₁₋₈alkyl,
(ii) C₁₋₈alkoxy,
(iii) C₁₋₈alkyl(C₁₋₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₃,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro,
(xiv) C₁₋₈alkyl(amino) optionally mono or disubstituted on amino with C₁₋₈alkyl,
(xv) C₁₋₈alkyl(aryl), and
(xvi) C₁₋₈alkoxy(aryl).

4. A compound according to claim 1, wherein the compound is a compound of Formula (V) 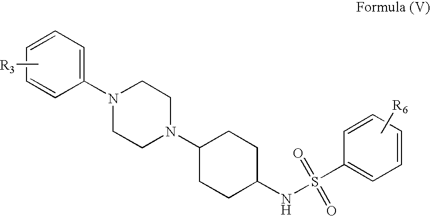
and pharmaceutically acceptable forms thereof, wherein
R₃ is up to four optionally present substituents independently selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) aryl,
(16) C₁₋₈alkyl(aryl),
(17) C₁₋₈alkoxy(aryl),
(18) C₃₋₈cycloalkyl,
(19) C₁₋₈alkyl(C₃₋₈cycloalkyl),
(20) C₁₋₈alkoxy(C₃₋₈cycloalkyl),
(21) heteroaryl,
(22) C₁₋₈alkyl(heteroaryl),
(23) heterocyclyl,
(24) C₁₋₈alkyl(heterocyclyl), and
(25) C₃₋₈cycloalkoxy,
wherein (15), (18), (21) and (23) and the aryl, C₃₋₈cycloalkyl, heteroaryl and heterocyclyl portions of (16), (17), (19), (20), (22), (24), and (25) are optionally substituted with from one to two substituents independently selected from the group consisting of
(i) C₁₋₈alkyl,
(ii) C₁₋₈alkoxy,
(iii) C₁₋₁₈alkyl(C₁₋₁₈alkoxy),
(iv) C₁₋₈alkyl(halogen)₁₋₁₇,
(v) C₁₋₈alkoxy(halogen)₁₋₁₇,
(vi) C₁₋₈alkyl(hydroxy)₁₋₁₇,
(vii) CO₂(C₁₋₈alkyl),
(viii) SO₂(C₁₋₈alkyl),
(ix) amino optionally mono- or di-substituted with C₁₋₈alkyl,
(x) cyano,
(xi) halogen,
(xii) hydroxy,
(xiii) nitro, and
(xiv) C₁₋₈alkyl(amino) optionally mono- or di-substituted on amino with C₁₋₈alkyl; and
R₆ is selected from the group consisting of
(1) C₁₋₈alkyl,
(2) C₁₋₈alkoxy,
(3) C₁₋₈alkyl(C₁₋₈alkoxy),
(4) C₁₋₈alkyl(halogen)₁₋₁₇,
(5) C₁₋₈alkoxy(halogen)₁₋₁₇,
(6) C₁₋₈alkyl(hydroxy)₁₋₃,
(7) CO₂(C₁₋₈alkyl),
(8) SO₂(C₁₋₈alkyl),
(9) amino optionally mono- or di-substituted with C₁₋₈alkyl,
(10) cyano,
(11) halogen,
(12) hydroxy,
(13) nitro,
(14) C₁₋₈alkyl(amino) optionally mono- or di-substituted on amino with C₁₋₈alkyl,
(15) C₁₋₈alkyl(aryl), and
(14) C₁₋₈alkoxy(aryl).

5. A compound according to claim 4, wherein the compound is selected from 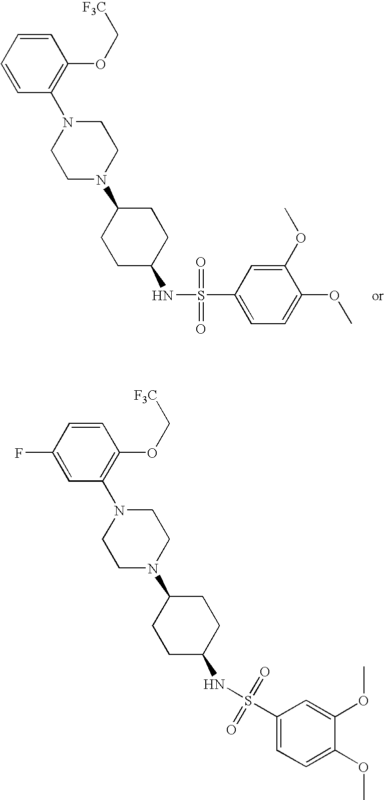
or pharmaceutically acceptable forms thereof, preferably difumarate salts thereof.

6. The compound of any of claim 1 to 5, wherein the compound is an isolated form thereof.

7. The compound of any of claim 1 to 6, wherein the form of said compound is a pharmaceutical composition or medicament comprising an effective amount of one or more of said compound.

8. The pharmaceutical composition of claim 7, wherein the composition further comprises an effective amount of the compound and a pharmaceutically acceptable carrier.

9. A process for preparing a pharmaceutical composition comprising the step of admixing a compound of any of claim 1 to 6 and a pharmaceutically acceptable carrier.

10. The pharmaceutical composition of any of claim 7 to 8, wherein the effective amount of the compound is in a range of from about 0.001 mg/kg to about 300 mg/kg of body weight per day.

11. A compound of any of claim 1 to 6 for use as a medicine.

12. A method for treating, ameliorating or preventing an α₁ₐ and/or α_{1d} adrenoreceptor mediated disorder or disease in a subject in need of such treatment, amelioration or prevention comprising administering to the subject a therapeutically or prophylactically effective amount of a compound of any of claim 1 to 6.

13. The method of claim 12, wherein the method further comprises treating Benign Prostatic Hyperplasia in a subject in need of such treatment comprising administering to the subject in need of such treatment a therapeutically effective amount of a compound of any of claim 1 to 6.

14. The method of claim 12, wherein the method further comprises treating Lower Urinary Tract Symptoms in a subject in need of such treatment comprising administering to the subject in need of such treatment a therapeutically effective amount of a compound of any of claim 1 to 6.

15. The method of any of claim 12 to 14, wherein the effective amount of the compound is from about 0.001 mg/kg/day to about 300 mg/kg/day.

16. The method of any of claim 12 to 14, wherein the method further comprises administering to the subject an effective amount of a combination product comprising one or more of the compound and at least one other therapeutic agent.

17. The method of claim 16, wherein the therapeutic agent is selected from a human testosterone 5-α reductase inhibitor, a 5-α reductase isoenzyme 2 inhibitor, an anti-antiandrogenic agent, an androgen receptor antagonist, a selective androgen receptor modulator, a urinary incontinence drug, an anti-muscarinic agent or a 5HT-receptor modulator.

18. The method of claim 16, wherein the therapeutic agent is selected from finasteride.

19. A process for preparing a compound of any of claim 1 to 6 comprising the steps of 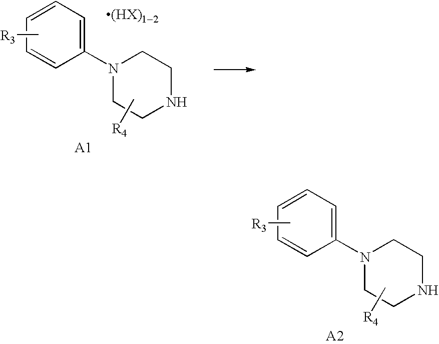
(a) reacting a Compound A1 in the presence of a base and a suitable solvent to provide a Compound A2; 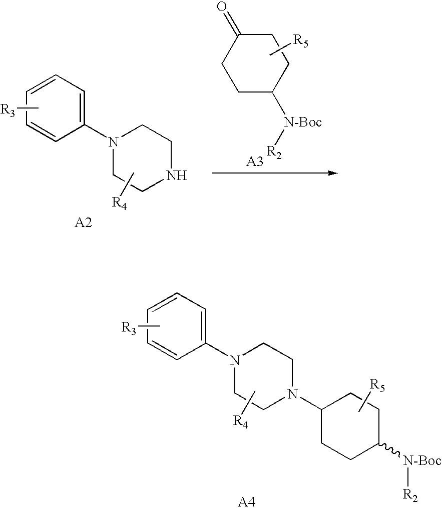
(b) reacting Compound A2 with a Compound A3 and a reducing agent in the presence of an optional acid catalyst in a suitable solvent to provide a Compound A4; 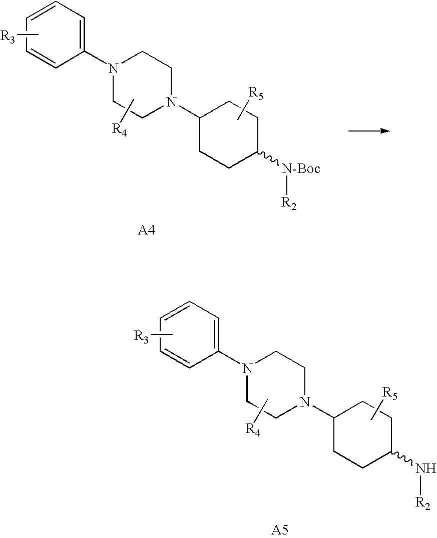
(c) reacting Compound A4 under acidic conditions in a suitable solvent to provide a deprotected Compound A5; 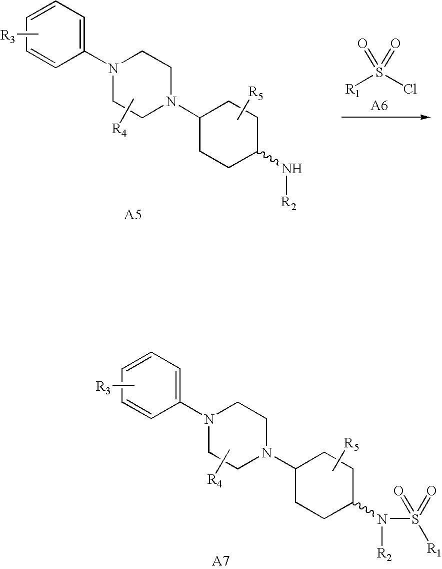
(d) reacting Compound A5 with a Compound A6 in the presence of a mild base in a suitable solvent to provide a Compound A7 as a racemic mixture; and 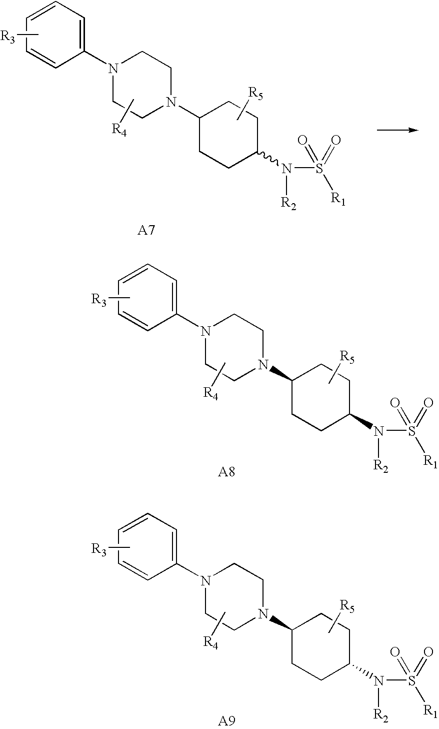
(e) separating the Compound A7 mixture in a suitable solvent to provide a cis isomer Compound A8 and a trans isomer Compound A9.
